(19) United States
(12) Patent Application Publication
(10) Pub. No.: US 2026/0355299 A1
(43) Pub. Date: Nov. 12, 2026

(54) SFTS VIRUS VACCINES

(71) Applicant: The Cleveland Clinic Foundation, Cleveland, OH (US)

(72) Inventors: Jae U. JUNG, Cleveland, OH (US); Chih-Jen LAI, Cleveland, OH (US); Dokyun KIM, Cleveland, OH (US)

(21) Appl. No.: 19/139,652

(22) PCT Filed: Dec. 14, 2023
(86) PCT No.: PCT/US2023/084094

§ 371 Date: Jun. 16, 2025

Related U.S. Application Data
(60) Provisional application No. 63/433,294, filed on Dec. 16, 2022.
(60) Provisional application No. 63/588,891, filed on Oct. 9, 2023.

(51) Int. Cl. A61K 39/12; A61K 9/00; A61K 39/00; A61K 39/39; A61P 31/14; C12N 7/00

(52) U.S. Cl. CPC A61K 39/12; A61K 9/0019; A61K 39/39; A61P 31/14; C12N 7/00; A61K 2039/53; A61K 2039/54; A61K 2039/545; A61K 2039/55516; A61K 2039/55555; A61K 2039/55566; A61K 2039/575; A61K 2039/6018; C12N 2760/12034; C12N 2760/12071

(57) ABSTRACT

Provided herein are compositions, systems, kits, and methods for immunizing a subject against severe fever with thrombocytopenia syndrome virus (SFTS virus) with a composition i) comprising a plurality of nanoparticles self-assembled from a plurality of fusion proteins that comprise: a) at least a portion of a Ferritin protein, and b) an immunogenic protein comprising at least a portion of: SFTS virus Gn and/or Gc envelope glycoprotein, or ii) polynucleotides encoding said fusion proteins (e.g., mRNA sequences present in lipid nanoparticles).

The present application claims priority to U.S. Provisional Applications 63/433,294 filed Dec. 16, 2022, and 63/588,891, filed Oct. 9, 2023, both of which are herein incorporated by reference in their entireties.

This invention was made with government support under AI152190 and AI140705 awarded by the National Institutes of Health. The government has certain rights in the invention.

### FIELD

Provided herein are compositions, systems, kits, and methods for immunizing a subject against severe fever with thrombocytopenia syndrome virus (SFTS virus) with a composition i) comprising a plurality of nanoparticles self-assembled from a plurality of fusion proteins that comprise: a) at least a portion of a Ferritin protein, and b) an immunogenic protein comprising at least a portion of: SFTS virus Gn and/or Gc envelope glycoprotein, or ii) polynucleotides encoding said fusion proteins (e.g., mRNA sequences present in lipid nanoparticles).

### BACKGROUND

Sever Fever with Thrombocytopenia Syndrome (SFTS) is caused by SFTSV (SFTS virus), also called Dabie bandavirus (DBV), and is characterized by clinical symptoms of fever, thrombocytopenia, leukopenia and multiple organ failure accompanying hemorrhage, and a mortality rate up to 30%. It was first reported in East Asia in 2009, and became endemic to the area. The main route of human transmission is tick bite, although there are reports of animal to human and human to human transmission. It has an especially high fatality rate in the elderly population, ranging from 12 to 30%. More than 8,500 cases have been reported, and fatal cases are primarily in the elderly population, thus strongly suggesting age-dependence. Asian Longhorned ticks transmit the virus and were first found in the US in 2017. The ticks transmitting SFTSV have spread to more than 19 states in the United States, thus indicating the possibility of an outbreak beyond East Asia. Therefore, there is an increasing need for a safe vaccine against SFTSV, especially for the aged population. Currently, there is no therapy or vaccine licensed. Recently, SFTSV has been listed as one of WHO and NIH's priority pathogens.

### SUMMARY

Provided herein are compositions, systems, kits, and methods for immunizing a subject against severe fever with thrombocytopenia syndrome virus (SFTS virus) with a composition i) comprising a plurality of nanoparticles self-assembled from a plurality of fusion proteins that comprise: a) at least a portion of a Ferritin protein, and b) an immunogenic protein comprising at least a portion of: SFTS virus Gn and/or Gc envelope glycoprotein, or ii) polynucleotides encoding said fusion proteins (e.g., mRNA sequences present in lipid nanoparticles).

In some embodiments, provided herein are compositions comprising: a plurality of fusion proteins, wherein each of the plurality of fusion proteins comprises: a) at least a portion of a Ferritin protein, and b) an immunogenic protein comprising at least a portion of: i) severe fever with thrombocytopenia syndrome virus (SFTS) virus Gn envelope glycoprotein and/or ii) SFTS virus Gc envelope glycoprotein, wherein the plurality of fusion proteins are self-assembled into a plurality of nanoparticles (or would self-assemble under appropriate conditions), and wherein the plurality of nanoparticles each display the immunogenic protein on their surfaces.

In particular embodiments, provided herein are methods of immunizing a subject (e.g., human subject) comprising: administering at least a portion of the plurality of nanoparticles described above and herein to a subject such that an immune response to the immunogenic protein is produced in the subject (e.g., such that SFTS virus neutralizing antibodies are generated in the subject).

In other embodiments, provided herein are methods of producing a plurality of nanoparticles comprising: a) culturing a plurality of cells in culture media, wherein the plurality of cells each comprise a vector comprising a nucleic acid sequence encoding a fusion protein that comprises: i) at least a portion of a Ferritin protein, and ii) an immunogenic protein comprising at least a portion of: i) severe fever with thrombocytopenia syndrome virus (SFTS) virus Gn envelope glycoprotein and/or ii) SFTS virus Gc envelope glycoprotein, wherein the culturing is under conditions such that each of the plurality of cells expresses a plurality of the fusion proteins that self-assemble into a plurality of nanoparticles, and wherein the plurality of nanoparticles display the immunogenic protein on their surfaces; and b) recovering the plurality of nanoparticles to generate a first composition.

In some embodiments, the methods further comprise purifying the first composition to substantially remove non-nanoparticle material, thereby generating a second composition. In additional embodiments, the methods further comprise mixing the second composition with an adjuvant.

In certain embodiments, provided here are nucleic acid molecules that encode any of the fusion proteins described herein, including in the figures.

In particular embodiments, the compositions further comprise an adjuvant. In other embodiments, the at least a portion of the Ferritin protein comprises at least 25 consecutive amino acids (e.g., 25 . . . 50 . . . 75 . . . 100 . . . 125 . . . 150 . . . or more) from a Ferritin protein. In some embodiments, the at least a portion of the Ferritin protein comprise at least 25 consecutive amino acids, or at least 100 consecutive amino acids, or at least about 150 consecutive amino acids, or the entire amino acid sequence; from an amino acid sequence selected from: SEQ ID NOs: 4, 5, 6, 7, and 8. In additional embodiments, the at least a portion of the Ferritin protein comprises an amino acid sequence at least about 90% identical, or at least 95% identical, or at least 99% identical, or 100% identical, to an amino acid sequence selected from SEQ ID NOs: 4, 5, 6, 7, and 8. In certain embodiments, the at least a portion of the Ferritin protein comprises an amino acid sequence selected from SEQ ID NOs: 4, 5, 6, 7, 8 or SEQ ID NOs: 4, 5, 6, 7, and 8 with one or two conservative amino acid changes. In some embodiments, the at least a portion of the Ferritin protein is a hybrid protein comprising at least a portion of a bullfrog (Rana catesbeiana) ferritin protein joined to at least a portion of a ferritin protein selected from the group consisting of a Helicobacter pylori ferritin protein and an Escherichia coli ferritin protein.

In particular embodiments, the at least a portion of the SFTS virus Gn envelope glycoprotein comprises at least 25, or at least 50, consecutive amino acids, or at least 100 consecutive amino acids, or at least about 200 consecutive amino acids (e.g., 50 . . . 100 . . . 125 . . . 150 . . . 175 . . . or 200 consecutive amino acids), or the entire amino acid sequence; from an amino acid sequence selected from: SEQ ID NOs: 2, 9, 11, 25, and 27, or SEQ ID NOs: 2, 9, 11, 25 and 27 with one or two conservative amino acid changes or end deletions. In other embodiments, the at least a portion of the SFTS virus Gc envelope glycoprotein comprises at least 25 consecutive amino acids, or at least 100 consecutive amino acids, or at least about 200 consecutive amino acids, or the entire amino acid sequence; from an amino acid sequence selected from: SEQ ID NOs: 3, 13, and 15, or SEQ ID NOs: 3, 13 and 15 with one or two conservative amino acid changes. In some embodiments, the fusion proteins further comprises a linker sequence.

In some embodiments, provided herein are compositions comprising: a polynucleotide encoding at least a portion of a fusion protein, wherein the fusion protein comprises: a) at least a portion of a Ferritin protein, and b) an immunogenic protein comprising at least a portion of: i) severe fever with thrombocytopenia syndrome virus (SFTS) virus Gn envelope glycoprotein and/or ii) SFTS virus Gc envelope glycoprotein. In certain embodiments, the polynucleotide is at least partially optimized for enhanced expression, reduced immunogenicity, increased stability, or a combination thereof.

In certain embodiments, the polynucleotide comprises at least 24 or 35 consecutive nucleotides from any of SEQ ID NOs: 1, 10, 12, 14, 16, 18, 20, 22, 24, 26, 31, or 33-40, wherein T is optionally replaced with U, and/or any of the nucleotides, including U, are replaced with a modified base. In some embodiments, the at least a portion of the Ferritin protein comprises at least 25 consecutive amino acids from a Ferritin protein. In further embodiments, the at least a portion of the Ferritin protein comprise at least 25 consecutive amino acids, or at least 100 consecutive amino acids, or at least about 150 consecutive amino acids, or the entire amino acid sequence, from an amino acid sequence selected from: SEQ ID NOs: 4, 5, 6, 7, and 8. In additional embodiments, the at least a portion of the Ferritin protein comprises an amino acid sequence at least about 90% identical, or at least 95% identical, or at least 99% identical, or 100% identical, to an amino acid sequence selected from SEQ ID NOs: 4, 5, 6, 7, and 8. In certain embodiments, the at least a portion of the Ferritin protein comprises an amino acid sequence selected from SEQ ID NOs: 4, 5, 6, 7, 8 or SEQ ID NOs: 4, 5, 6, 7, and 8 with one or two conservative amino acid changes.

In some embodiments, the at least a portion of the Ferritin protein is a hybrid protein comprising at least a portion of a bullfrog ferritin protein joined to at least a portion of a ferritin protein selected from the group consisting of a Helicobacter pylori ferritin protein and an Escherichia coli ferritin protein. In other embodiments, the at least a portion of the SFTS virus Gn envelope glycoprotein comprises at least 25, or at least 50, consecutive amino acids, or the entire amino acid sequence; from an amino acid sequence selected from: SEQ ID NOs: 2, 9, 11, 25, and 27, or SEQ ID NOs: 2, 9, 11, 25, and 27 with one or two conservative amino acid changes or end deletions. In further embodiments, the at least a portion of the SFTS virus Gc envelope glycoprotein comprises at least 25 consecutive amino acids, or at least 100 consecutive amino acids, or at least about 200 consecutive amino acids, or the entire amino acid sequence; from an amino acid sequence selected from: SEQ ID NOs: 3, 13, and 15, or SEQ ID NOs: 3, 13 and 15 with one or two conservative amino acid changes. In particular embodiments, the fusion protein further comprises a linker sequence.

In some embodiments, the polynucleotide comprises RNA. In further embodiments, the polynucleotide is partially or fully human codon optimized, or partially or fully HSV-1 glycoprotein B codon optimized, and is optionally RNA. In other embodiments, the polynucleotide further comprises or encodes: a 5′ untranslated region (UTR), a 5′ cap, a 3′ UTR, an IRES, a 3′ tailing sequence, or any combination thereof. In other embodiments, the 3′ tailing sequence comprises a polyA tail, a polyG quartet, a stem loop sequence, a triple helix forming sequence, a tRNA-like sequence, or any combination thereof.

In particular embodiments, the polynucleotide comprises at least one chemically modified nucleotide (e.g., one, two, 25% . . . 50% . . . 75% or 100% of the uracils). In additional embodiments, the at least one chemically modified nucleotide comprises a modified uracil. In other embodiments, at least 60% of the uracils in the polynucleotide are chemically modified. In further embodiments, the at least one chemically modified nucleotide comprises 5-methylcytosine or N1-methylpseudouridine (m1Ψ). In some embodiments, the polynucleotide comprises a nucleotides sequence: i) having at least 75%, or 85%, or 95% identity to SEQ ID NOs: 1, 10, 12, 14, 16, 18, 20, 22, 24, 26, 31, or 33-40, or a complement or reverse complement thereof, wherein T may be replaced by U or a modified U, and/or any of the nucleotides, including U, are replaced with a modified base.

In some embodiments, provided herein are vaccines comprising a composition as described above or herein, and at least one adjuvant (for example, AddaVax or MF59), a delivery vehicle, or a combination thereof. In certain embodiments, the delivery vehicle comprises a lipid nanoparticle encapsulating the composition. In particular embodiments, the lipid nanoparticle comprises a cationic lipid, a neutral and/or non-cationic lipid, a sterol, or any combination thereof. In some embodiments, the non-cationic lipid comprises a phospholipid. In certain embodiments, the sterol comprises cholesterol or a modification or ester thereof. In some embodiments, the lipid nanoparticle comprises a polyethylene glycol (PEG)-lipid conjugate.

### DESCRIPTION OF THE FIGURES

FIG. 1A shows a schematic representation of SFTS virus M segment precursor protein (1073 amino acids), which is process in cells (at the cleavage site) to yield envelope protein Gn and envelope protein Gc. FIG. 1B shows a schematic representation of the head of Gn fused to ferritin protein (Gn Head-FT or GnH-FT). FIG. 1C shows a schematic representation of the head+ stem of Gn fused to ferritin protein (Gn Head+Stem-FT or GnHS-FT). FIG. 1D shows a schematic representation of Domains 1-3 of Gc fused to ferritin protein (Gc Domains 1-3-FT or GcD-FT). FIG. 1E shows a schematic representation Gc Ecto-FT or GcE-FT).

FIG. 2 shows exemplary methods for expression and purification of Gn/Gc-ferritin nanoparticles using plasmid vectors encoding, for example, the constructs shown in FIG. 1. For example, HEK 293T cells in serum free media are transfected with such plasmids in a dish; the supernatant is collected subjected to tangential flow filtration; collect the retentate; and perform size exclusion chromatograph in columns to isolate the Gn-ferritin or Gc-ferritin nanoparticles.

FIG. 3: A) Computer-assisted 3D modeling of an empty ferritin (eFT) (top panel), and Cryo-Electron microscopy of empty ferritin (eFT) (lower panel). B) Computer-assisted 3D modeling of sGnH-FT (top panel), and Cryo-Electron microscopy of sGnH-FT. Comparing A bottom panel with B bottom panel, SFTSV Gn Head domain is carried on the surface of the ferritin nanoparticle, appearing as the white halo.

FIG. 4 shows, after the cell are transfected with plasmid vectors encoding, for example, the constructs shown in FIG. 1, three exemplary methods that can be performed with a Gn or Gc antibody to determine if Gn or Gc is presented on the nanoparticle surfaces as on SFTSV surface, including: immunoprecipitation, immunoblot, and ELISA.

FIG. 5: (A) shows the immunization, blood draw, and sacrifice schedule for mice immunized with the soluble GnH-FT nanoparticles. Five Balb/C (Female mice) per group were intramuscularly immunized (hind leg) with eFT (empty ferritin nanoparticles) (3.3 μg) and sGnH-FT (1 μg, 5 μg and 10 μg). (B) ELISA with sGnH-10His coating to measure reciprocal antibody titer specifically against sGnH. (C) ELISA with sGnH-FT coating to measure reciprocal antibody titer against sGnH-FT. (D) ELISA with eFT coating to measure reciprocal antibody titer against eFT.

FIG. 6A the amino acid sequence of Gn envelope protein of SFTS virus (SEQ ID NO: 2). FIG. 6B the amino acid sequence of Gc envelope protein of SFTS virus (SEQ ID NO: 3). These sequence, or immunogenic portions thereof, may be employed with the Ferritin constructs herein.

FIG. 7A shows the amino acid sequence of Helicobacter pylori Ferritin protein (SEQ ID NO: 4). FIG. 7B shows the amino acid sequence of Escherichia coli Ferritin protein (SEQ ID NO: 5).

FIG. 8A shows the amino acid sequence of Rana catesbeiana (Bull Frog) Ferritin protein (SEQ ID NO:6). FIG. 8B shows the amino acid sequence of H. pylori ferritin-bullfrog ferritin fusion (SEQ ID NO:7).

FIG. 9 shows the amino acid sequence of E. coli ferritin-bullfrog ferritin fusion (SEQ ID NO: 8).

FIG. 10A shows the nucleic acid sequence encoding the GnH (Gn head group) protein employed in Example 1 (SEQ ID NO:1). FIG. 10B shows the amino acid sequence for GnH (Gn head group) protein (SEQ ID NO:9).

FIG. 11A shows the nucleic acid sequence encoding the Gn Head+Stem (GnHS) protein (SEQ ID NO:10). FIG. 11B shows the amino acid sequence for the Gn Head+Stem (GnHS) protein (SEQ ID NO:11).

FIG. 12A shows the nucleic acid sequence encoding the Gc Domains 1-3 (GcD) protein (SEQ ID NO:12). FIG. 12B shows the amino acid sequence for the Gc Domains 1-3 (GcD) protein (SEQ ID NO:13).

FIG. 13A shows the nucleic acid sequence encoding the Gc Ecto (GcE) protein (SEQ ID NO: 14). FIG. 13B shows the amino acid sequence for the Gc Ecto (GcE) protein (SEQ ID NO: 15).

FIG. 14A shows the nucleic acid sequence encoding the Gn head group-FT (GnH-FT) fusion protein employed in Example 1 (SEQ ID NO:16). FIG. 14B shows the amino acid sequence for Gn head group-FT (GnH-FT) fusion protein (SEQ ID NO:17).

FIG. 15A shows the nucleic acid sequence encoding the Gn Head+Stem-FT (GnHS-FT) fusion protein (SEQ ID NO:18). FIG. 15B shows the amino acid sequence for the Gn Head+Stem-FT (GnHS-FT) fusion protein (SEQ ID NO:19).

FIG. 16A shows the nucleic acid sequence encoding the Gc Domains 1-3-FT (GcD-FT) fusion protein (SEQ ID NO:20). FIG. 16B shows the amino acid sequence for the Gc Domains 1-3-FT (GcD-FT) fusion protein (SEQ ID NO:21).

FIG. 17A shows the nucleic acid sequence encoding the Gc Ecto-FT (GcE-FT) fusion protein (SEQ ID NO:22). FIG. 17B shows the amino acid sequence for the Gc Ecto-FT (GcE-FT) fusion protein (SEQ ID NO:23).

FIG. 18A shows the nucleic acid sequence of Domain 2 of Gn (SEQ ID NO:24). FIG. 18B shows the amino acid sequence of Domain 2 of Gn (SEQ ID NO:25). FIG. 18C shows the nucleic acid sequence of Domain 3 of Gn (SEQ ID NO:26). FIG. 18D shows the amino acid sequence of Domain 3 of Gn (SEQ ID NO:27).

FIG. 19. Molecular design and biochemical and antigenic characterization of ferritin nanoparticles (FT) and DBV Gn Head-ferritin (GnH-FT) nanoparticles. (A) Schematic representation of GnH-FT based on previously solved structures and domains of DBV Gn and Gc. The construct was transfected to HEK293T cells to collect the cell supernatant at 72 hours after transfection for purification. (SP: signal peptide, TM: transmembrane domain) (B, C) Size exclusion chromatograms to purify FT (B) and GnH-FT (C) using Superdex 200. Increase 10/300 GL and Superose6 Increase 10/300 GL columns, respectively, on Bio-Rad NGC chromatography system. Fractions corresponding to the colored arrows were separately collected for further analyses. (D, E) Fractions from size exclusion chromatograms of FT and GnH-FT were further analyzed by gradient (7%-20%) SDS-PAGE and Coomassie Brilliant blue staining. Fractions corresponding to the black and red arrows from FT and GnH-FT purifications were loaded to SDS-PAGE gel without boiling (“NB”) and with boiling (“B”) to characterize head-mediated disassembly of the 24-mer nanoparticle. Intact FT nanoparticle and GnH-FT nanoparticle each has expected molecular weight of approximately 432 kDa and 1,560 kDa. Dissembled FT and GnH-FT monomer each has expected molecular weight of 18 kDa and 65 kDa. (F) Western-blot analysis of different fractions collected from GnH-FT size exclusion chromatogram. In mouse monoclonal antibody recognizing DBV Gn Head region was used to detect GnH-FT subunit monomers.

FIG. 20. Prediction and observed structures of FT and GnH-FT nanoparticles. (A, B) Computer-assisted 3D model of the FT nanoparticles (A) and GnH-FT nanoparticles (B) based on previously solved structures of DBV Gn (PDB: 5Y11) and FT nanoparticles (PDB: 3EGM). (C, D) Cryo-electron microscopy (cryo-EM) of FT (C) and GnH-FT (D) nanoparticles. (E, F) Representative 2D class averages of FT (E) and GnH-FT (F). The white halo surrounding the FT nanoparticle core in (F) is attributable to the highly flexible linker and DBV Gn head domain.

FIG. 21. Immunization with GnH-FT elicits humoral and cellular immunity in vivo. (A) Timeline for mouse immunization and blood collection. Six BALB/c mice per antigen group were intramuscularly immunized at hind leg with 3.3 μg of FT or 1 μg, 5 μg, or 10 μg of GnH-FT. 3.3 μg of FT is equimolar to 10 μg of GnH-FT. (B) Reciprocal IgG titer measured by ELISA using purified GnH-10His protein coated. Sera from blood samples collected at weeks 0, 2, 3, 5, 6, and 8 were used to quantify total IgG recognizing DBV Gn Head. Endpoint titer values are presented in log 10 values. The asterisks represent statistical significance of endpoint titer between mice immunized with GnH-FT and FT evaluated with one-way ANOVA with Dunnett multiple comparison test. (C) Neutralizing antibody titer measured by luciferase assay using rVSV-DBV G carrying luciferase gene. Sera from blood samples collected at weeks 0, 2, 5, and 8 were used to quantify reciprocal IC₅₀ titer to represent induction of neutralizing antibody upon immunization with different antigens. One-way ANOVA with Dunnett multiple comparison test was performed. (D) ELISpot assays were performed to detect DBV Gn Head-specific T cells secreting IFN-γ by ex vivo stimulation of whole splenocytes with pool of overlapping peptides (OLP). Data represents the number of spot-forming units (SFUs) per 1 million splenocytes. One-way ANOVA with Dunnett multiple comparison test was used to evaluate statistical significance. (E) Intracellular cytokine staining assay was performed to test for activity of cellular immunity induced upon immunization with the antigens. Splenocytes were stimulated ex vivo as for ELISpot assay, treated with protein transport inhibitor, then stained for anti-TNF-α antibodies or IL-2 antibodies. Two-tailed, unpaired t-test was performed to evaluate statistical significance. *P<0.05, **P<0.01, ***P<0.001 and ****P<0.00001.

FIG. 22. Aged ferrets form humoral immunity upon immunization with GnH-FT. (A) Timeline for aged ferret immunization, lethal DBV challenge, and organ harvest. Based on the effective induction of humoral and cellular immunity in mouse model, aged ferrets were immunized with total three doses of 15 μg GnH-FT or FT. Each antigen group had 12 ferrets. Aged ferrets were challenged with 107.6 746 TCID50/mL of DBV for infection with lethal dose at 2 weeks after the last booster immunization, then monitored for clinical symptoms of SFTS. At 2, 4, and 6 days post the challenge, three ferrets per antigen group were sacrificed to harvest serum, spleen, liver, and kidney for organ virus titration to test for acceleration in viral clearance. (B) Reciprocal IgG titer measured by ELISA using blood samples collected at days −42, −28, −14 and 0 to characterize humoral immunity induced by immunization with FT or GnH-FT. Optical density (OD) was measured with a spectrometer (VarioSkan, Thermo) at detection wavelength of 450 nm. The asterisks represent statistical significance of OD measurements from ferrets immunized with GnH-FT to ferrets immunized with FT evaluated with one-way ANOVA with Dunnett multiple comparison test. (C) Neutralizing antibody response to DBV elicited by immunization with FT or GnH-FT was characterized as FRNT50 from blood samples collected at days −28, −14, and 0 to represent time points of 2 weeks after the 1st, 2nd, and 3^{rd} vaccination. The asterisks represent statistical significance of FRNT50 from ferrets immunized with GnH-FT to ferrets immunized with FT evaluated with one-way ANOVA with Dunnett multiple comparison test. *P<0.05, **P<0.01, ***P<0.001 and ****P<0.00001.

FIG. 23. Protective immunity elicited by GnH-FT nanoparticle immunization against lethal DBV challenge in aged ferrets (A, B, C) Body weight (A), survival curve (B) and temperature (C) of the aged ferrets were observed for 14 days from the lethal challenge. Body weight and temperature are presented as mean±SEM and statistical significance was analyzed by one-way ANOVA with Dunnett multiple comparison test. Statistical significance of survival across the antigens was analyzed with 2-tailed Mantel-Cox method. (D, E) White blood cell (WBC, D) and platelet (E) counts were measured from blood samples collected for 14 days after the lethal challenge. Data are presented as box plots with the upper (75%) and lower (25%) quartiles, the horizontal line (median) and whiskers (maximum and minimum). Statistical significance across the antigens was evaluated by a two-tailed unpaired t-test. *P<0.05, **P<0.01, ***P<0.001 and ****P<0.00001.

FIG. 24. Viral titration from serum, spleen, liver, and kidney Organs were harvested at days 2, 4, and 6 from three animals at each time point. DBV titer values from serum (A), spleen (B), liver (C), and kidney (D) were measured with real-time PCR. Data are shown as mean±SEM. The asterisks indicate statistical significance compared between ferrets immunized with FT nanoparticle and GnH-FT nanoparticle from the two-tailed, unpaired t-test. *P<0.05, **P<0.01, ***P<0.001 and ****P<0.00001.

FIG. 25. Expression of Gn-H and Gn-H-FT from transfection with DNA and in vitro transcribed mRNA encoding SFTSV Gn-H and Gn-H-FT. (A) Molecular design of mRNAs encoding SFTSV Gn-H and Gn-H-FT. SFTSV Gn-H was codon-optimized to human codon usage and cloned into a vector carrying 5′ UTR, signal peptide, and 3′ UTR. N1-Methyl-PseudoUTP and 5′ cap analog was incorporated during in vitro transcription using the linearized DNA template. mRNA constructs were enzymatically polyadenylated to generate functional mRNAs for encapsulation into lipid nanoparticle (LNP). SP: signal peptide. (B) RNA gel electrophoresis on MOPS/formaldehyde gel. mRNA without (“C”) and with polyadenylation (“+Poly A”) were analyzed for their sizes by RNA gel electrophoresis. The left panel shows mRNA encoding sGn-H, and the right panel shows mRNA encoding sGn-H-FT. The size of sGn-H is: 960 base pairs, and the size of linker and FT is 555 base pairs. “M” stands for size marker. (C) Western blot analysis of supernatant from HEK293T cells transfected with sGn-H and sGn-H-FT expression vectors using a mouse monoclonal antibody recognizing SFTSV Gn-H. The control (“ctrl”) lane stands for mock transfection. The “Boiled” panel represents an additional step of boiling at 95 °C for 10 minutes to disassemble the Gn-H-FT nanoparticle to monomers. (D) Western blot analysis of whole cell lysate (WCL) and supernatant from HEK293T cells transfected with polyadenylated sGn-H and sGn-H-FT mRNAs using a mouse monoclonal antibody recognizing SFTSV Gn-H. The “Boiled” and “non-boiled” panels are described in FIG. 25C. The control (“ctrl”) lane stands for mock transfection. The expected molecular weight of Gn-H is 45 kDa, Gn-H-FT monomer is 65 kDa, Gn-H-FT nanoparticle is 1,560 kDa, and vinculin is 124 kDa.

FIG. 26. Immunization and sample collection timeline of sGn-H and sGn-H-FT-immunized BALB/c mice. (A) Timeline of animal immunization with the mRNA vaccine candidates and blood collection. 6-8 weeks old BALB/c mice were immunized twice with 1 μg of sGn-H or sGn-H-FT at weeks 0 and 3. Blood was collected at weeks 0, 2, 5, 9, 12, and 15 to harvest sera to characterize antibody formation at the time points and to characterize the potential maintenance of neutralizing antibody titer over an extended period. (B) Reciprocal serum endpoint titer measured by ELISA to characterize total IgG titer recognizing SFTSV Gn-H. ELISA plates were coated with purified sGn-H-10His protein. Sera from the blood collections were serially ten-fold diluted, and endpoint titer values are demonstrated as log 10. Asterisks represent statistical significance between animals immunized with sGn-H mRNA vs sGn-H-FT mRNA. “Ctrl” stands for immunization with PBS at the same volume as sGn-H and sGn-H-FT immunization. (C) Neutralizing antibody titer measured by pseudovirus neutralization assay using rVSV-SFTSV G-Luc. The titer values are depicted as reciprocal IC50 titer in log 10 values. Numbers on top of the histogram represent the geometric mean titer and fold difference of the titer from mice immunized with Gn-H vs Gn-H-FT mRNAs. The dotted line represents the limit of detection. *stands for p-value <0.05, **stands for p-value <0.01, ***stands for p-value <0.001, and ns stands for not significant.

FIG. 27. Mice immunized with Gn-H mRNA and Gn-H-FT mRNA are fully protected from lethal SFTSV infection. (A) Timeline for the immunization of 6 to 8-weeks old type I Interferon receptor (Ifnar1) knockout (A129) mice. The mice were immunized with 3 μg of mRNA encoding sGn-H or sGn-H-FT twice at weeks 0 and 3 and infected with 2E4 pfu of SFTSV for lethal infection. Mice were monitored for clinical symptoms of body weight loss and fatality for 15 days after the infection. (B) Fatality of the immunized and challenged mice for 15 days after the infection. Data are presented as a percent survival over the period. (C) Body weight curve of the immunized and challenged mice for 15 days after the infection. Data are presented as % body weight change from 100% as the initial body weight at the time of viral infection. *stands for p-value <0.05, **stands for p-value <0.01, ***stands for p-value <0.001, and ns stands for not significant.

FIG. 28A shows the mRNA sequence (SEQ ID NO:31) of the fusion protein used in Example 3. FIG. 28B shows the mRNA signal peptide employed.

FIG. 29A shows an exemplary human codon optimized sGn-Head DNA sequence, labelled SEQ ID NO:33. FIG. 29B shows an exemplary human optimized sGn-Head RNA sequence, labelled SEQ ID NO:34.

FIG. 30A shows an exemplary human codon optimized sGn-Head-Ferritin DNA sequence, labelled SEQ ID NO:35. FIG. 30B shows an exemplary human codon optimized sGn-Head-Ferritin RNA sequence, labelled SEQ ID NO:36.

FIG. 31A shows an exemplary Herpesvirus Simplex-1 (HSV-1) glycoprotein B codon optimized sGn-Head DNA sequence labelled SEQ ID NO:37. FIG. 31B shows an exemplary HSV-1 glycoprotein B codon optimized sGn-Head RNA sequence labelled SEQ ID NO:38.

FIG. 32A shows an exemplary HSV-1 glycoprotein B codon optimized sGn-Head-Ferritin DNA sequence labelled SEQ ID NO:39. FIG. 32B shows an exemplary HSV-1 glycoprotein B codon optimized sGn-Head-Ferritin RNA sequence labelled SEQ ID NO:40.

FIG. 33 shows how viral glycoprotein codon optimization was applied to an mRNA vaccine design for Severe Fever with Thrombocytopenia Syndrome Virus (SFTSV). An mRNA vaccine encoding SFTSV Gn head alone (sGn-H) (SEQ ID NO:38) or Gn head with ferritin (sGn-H-FT) (SEQ ID NO:40) was designed such that the codon usage for the sGn-H portion was optimized to the usage pattern from HSV-1 glycoprotein B (gB). A comparison of codon usage for SFTSV Gn-head vs. HSV-1 gB is shown in FIG. 33. Codon usage was analyzed using the graphical codon usage analyzer (GCUA) software and Prism9 software. Codon optimized sequences were synthesized in vitro.

FIG. 34 shows increased SFTSV Gn-head/ferritin antigen expression was observed by western blot of cell lysates following HEK293T mRNA transfection upon codon optimization of the Gn-head portion to HSV-1 gB compared to human codon optimization (FIG. 34A. Expression was enhanced even more following mRNA nanoparticle (LNP) transfection (FIG. 34B). Specifically, this figure shows Western blot analysis of whole cell lysate from HEK293T cells transfected with sGn-H (human codon optimized), sGn-H (HSVgB codon optimized), sGN—H-FT (human codon optimized), and sGn-H-FT (HSVgB codon optimized) mRNAs (34A) or LNPs (34B) using a mouse monoclonal antibody recognizing SFTSV Gn-H.

### DETAILED DESCRIPTION

Provided herein are compositions, systems, kits, and methods for immunizing a subject against severe fever with thrombocytopenia syndrome virus (SFTS virus) with a composition i) comprising a plurality of nanoparticles self-assembled from a plurality of fusion proteins that comprise: a) at least a portion of a Ferritin protein, and b) an immunogenic protein comprising at least a portion of: SFTS virus Gn and/or Gc envelope glycoprotein, or ii) polynucleotides encoding said fusion proteins (e.g., mRNA sequences present in lipid nanoparticles).

SFTSV encodes two envelope glycoproteins (Gn and Gc). Gn, for example, has been shown to contain major neutralization epitopes. In work conducted during the development of embodiments herein, we have fused the head region of Gn to self-assembling ferritin for enhanced antigen presentation and have purified the Gn head nanoparticles for use as a vaccine (e.g., in humans).

In certain embodiments, the Gn or Gc-Ferritin nanoparticles are provided in compositions that further comprises an adjuvant. Any suitable adjuvant may be employed. Examples of adjuvants include, but are not limited to, Adda Vax, aluminum hydroxide, aluminum phosphate, and aluminum potassium sulfate.

The nanoparticle vaccine compositions herein may be introduced into a subject by any suitable route, such as intramuscular, intradermal, subcutaneous, subcutaneous, transdermal, or intravenous. In certain embodiments, the nanoparticle vaccine compositions are injected into the subject via intramuscular injection.

The present disclosure is not limited by the type of Ferritin protein employed. In certain embodiments, the Gn or Gc proteins (or immunogenic portions thereof) are joined (e.g., directly or via linker or other moiety) to at least 25 contiguous amino acids, at least 50 contiguous amino acids, at least 75 contiguous amino acids, at least 100 contiguous amino acids, or at least 150 contiguous amino acids from an amino acid sequence selected from the group consisting of SEQ ID NO: 4, SEQ ID NO:5 SEQ ID NO:6, SEQ ID NO:7, SEQ ID NO:8, wherein the fusion protein is capable of self-assembling into nanoparticles. In certain embodiments, the Gn or Gc proteins (or immunogenic portions thereof) are joined (e.g., directly or via linker or other moiety) to a protein that is at least 85%, at least 90%, at least 95%, at least 97% at least 99% identical to an amino acid sequence selected from the group consisting of SEQ ID NO:4, SEQ ID NO:5, SEQ ID NO: 6, SEQ ID NO:7, SEQ ID NO:8. Amino acid sequences from representative ferritin proteins of the present disclosure are disclosed herein as SEQ ID NO:4 (H. pylori ferritin), SEQ ID NO: 5 (E. coli ferritin), SEQ ID NO:6 (bullfrog ferritin), SEQ ID NO:7 (H. pylori ferritin-bullfrog ferritin fusion) and SEQ ID NO:8 (E. coli ferritin-bullfrog ferritin fusion).

In certain embodiments, sequences within the Gn/Gc fusion-Ferritin fusion protein are directly joined. In some embodiments, one may employ linkers, spacers or other types of sequences.

In certain embodiments, proteins of the present disclosure are encoded by nucleic acid molecules and are expressed by nucleic acid constructs. As used herein a nucleic acid construct is a recombinant expression vector (i.e., a vector linked to a nucleic acid molecule encoding a protein) such that the nucleic acid molecule can effect expression of the protein when the nucleic acid construct is administered to, for example, a subject or an organ, tissue or cell. The vector also enables transport of the nucleic acid molecule to a cell within an environment, such as, but not limited to, an organism, tissue, or cell culture. The nucleic acid construct can be DNA, RNA or variants thereof. The vector can be, for example, a DNA plasmid, a viral vector, or other vector. Examples of such vectors include, for example, cytomegalovirus (CMV), retrovirus, adenovirus, adeno-associated virus, herpes virus, vaccinia virus, poliovirus, or any other DNA or RNA virus vector. The nucleic acid molecules herein may be functionally linked to a promoter. In certain embodiments, provided herein is a cell that includes the foregoing nucleic acid molecule.

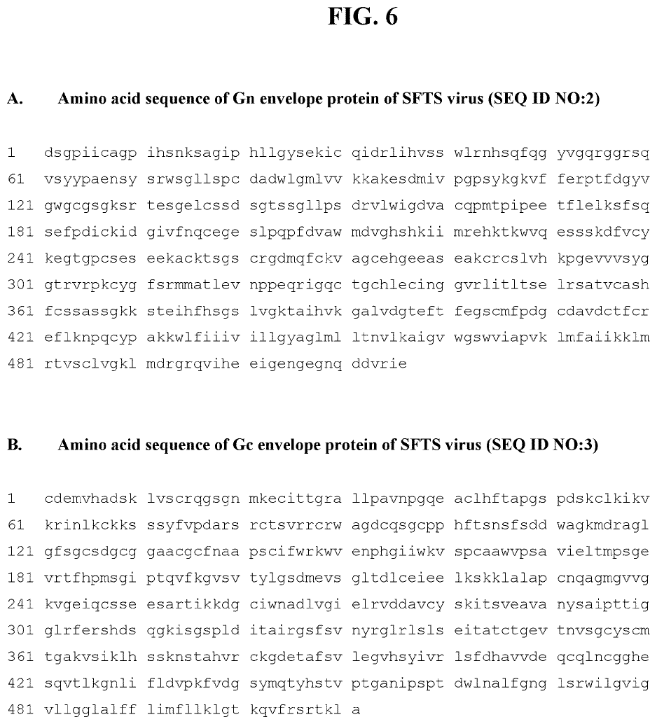

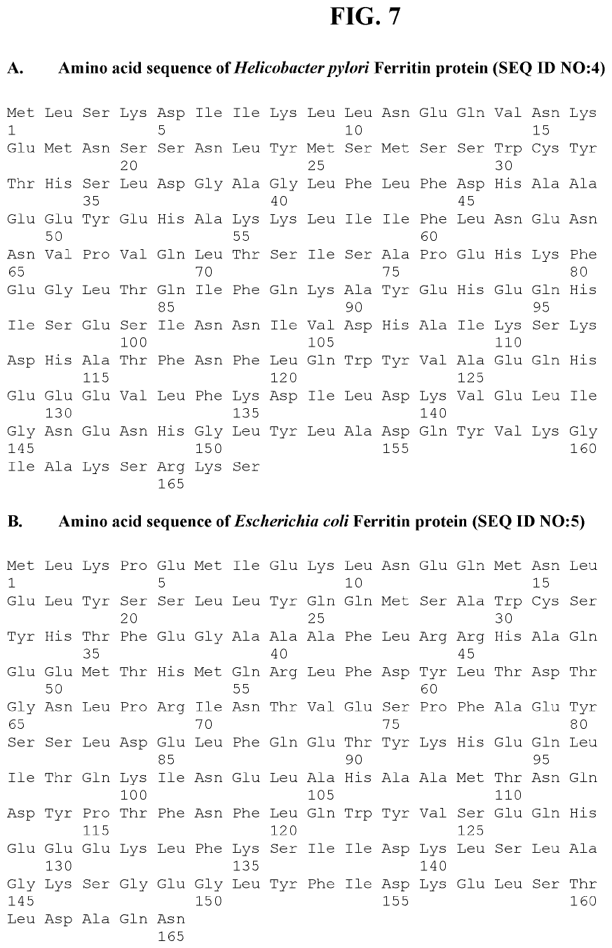

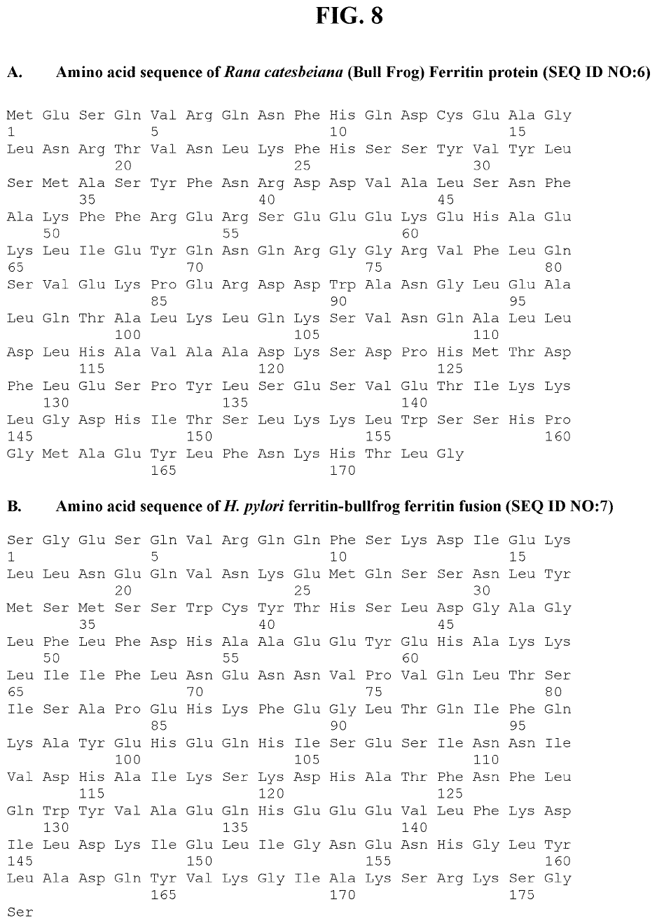

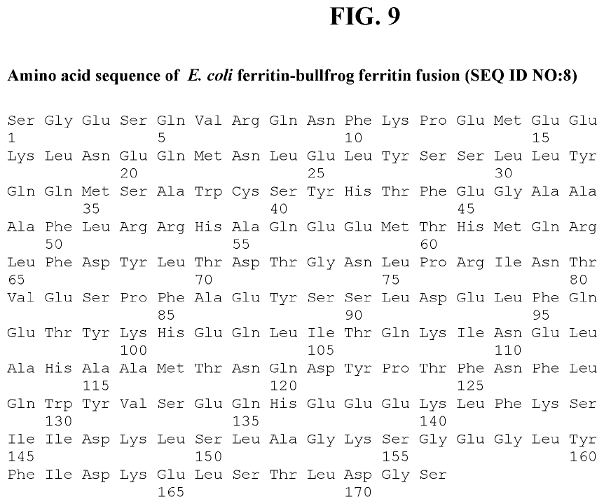

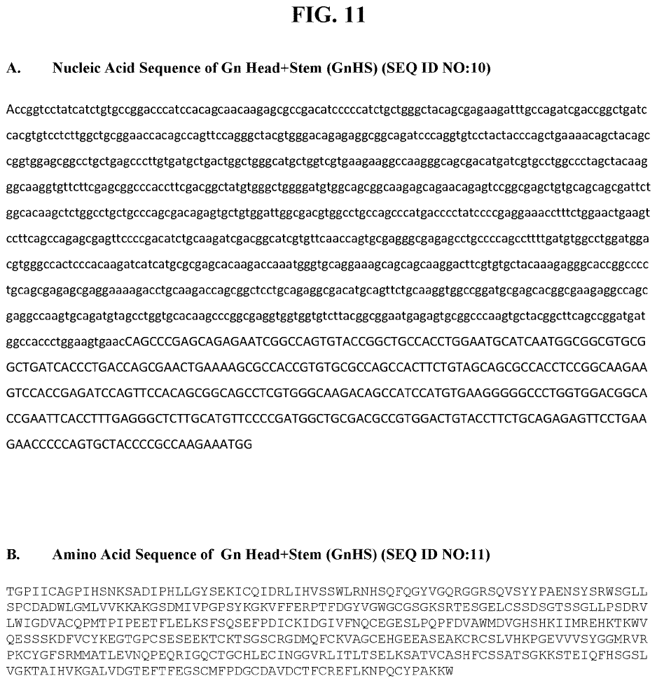

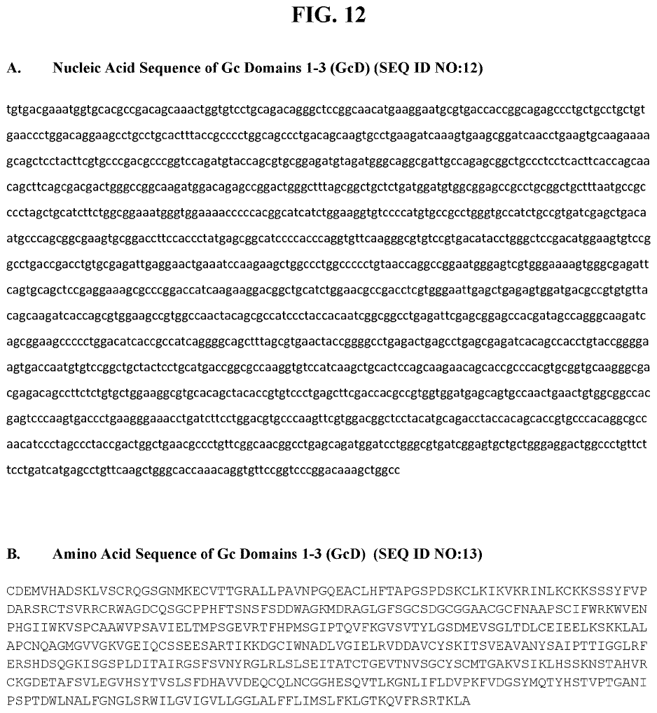

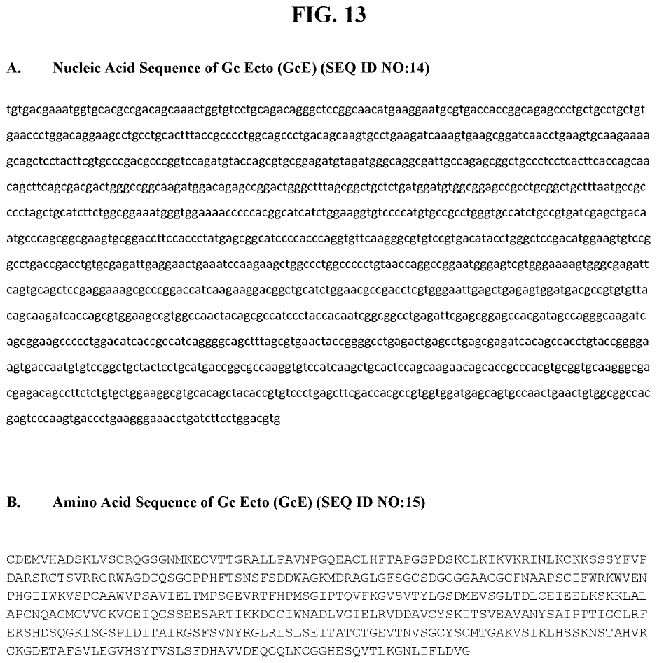

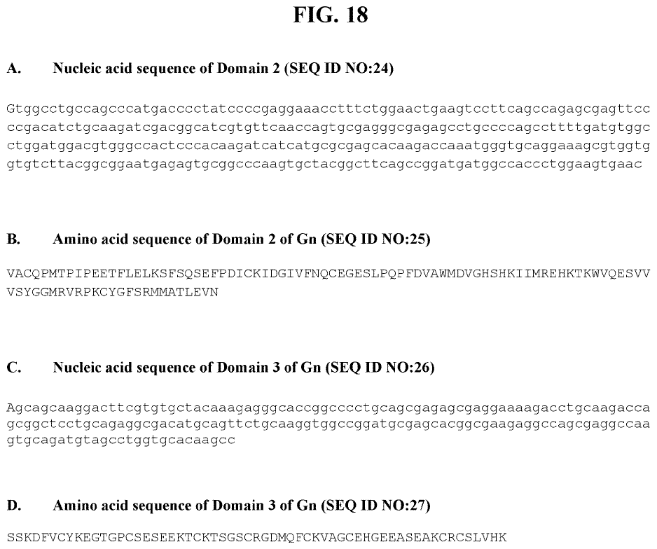

In certain embodiments, the Gn or Gc envelope proteins employed in the fusion protein may be full-length (see, e.g., FIG. 6) or portions thereof that are capable of eliciting an immune response in a subject. Examples of particular Gn or Gc proteins (and nucleic acid sequences encoding such proteins) are shown in FIGS. 6, 10-13, and 18.

In some embodiments, variants of such amino acid and nucleic acid sequences may be employed. A variant refers to a protein, or nucleic acid molecule, the sequence of which is similar, but not identical to, a reference sequence, wherein the activity of the variant protein (or the protein encoded by the variant nucleic acid molecule) is not significantly altered. These variations in sequence can be naturally occurring variations or they can be engineered through the use of genetic engineering technique known to those skilled in the art. Examples of such techniques are found in Sambrook J, Fritsch E F, Maniatis T et al., in Molecular Cloning-A Laboratory Manual, 2nd Edition, Cold Spring Harbor Laboratory Press, 1989, pp. 9.31-9.57), or in Current Protocols in Molecular Biology, John Wiley & Sons, N.Y. (1989), 6.3.1-6.3.6, both of which are incorporated herein by reference in their entirety. With regard to variants, any type of alteration in the amino acid, or nucleic acid, sequence is permissible so long as the resulting variant protein retains the ability to, for example, elicit neutralizing antibodies against an SFTS virus. Examples of such variations include, but are not limited to, deletions, insertions, substitutions and combinations thereof. For example, with regard to proteins, it is well understood by those skilled in the art that one or more (e.g., 2, 3, 4, 5, 6, 7, 8, 9 or 10), amino acids can often be removed from the amino and/or carboxy terminal ends of a protein without significantly affecting the activity of that protein. Similarly, one or more (e.g., 2, 3, 4, 5, 6, 7, 8, 9 or 10) amino acids can often be inserted into a protein without significantly affecting the activity of the protein.

In some embodiments, the polynucleotides or mRNAs herein encoding the fusion proteins herein, comprise at least one chemical modification or chemically modified base, nucleoside, or nucleotide. The chemical modifications may comprise any modification which is not naturally present in said RNA or any naturally-occurring modification of adenosine (A), guanosine (G), uridine (U), or cytidine (C) ribonucleosides. For example, a single polynucleotide or mRNA may include both naturally-occurring and non-naturally-occurring modifications. Chemical modifications may be located in any portion of the polynucleotide or mRNA molecule and the polynucleotide or mRNA molecule may contain any percentage of modified nucleosides (1-100%, such as at least 20% . . . at least 40% . . . or at least 60%). In some embodiments, every particular base or nucleoside may be modified (e.g., every uridine is a modified uridine). In some embodiments, at least 20%, or 50%, or 80% of any single nucleotide (e.g., uracil) in the of the polynucleotide or mRNA is chemically modified. In some embodiments, a particular modification is used for every particular type of nucleoside or base (e.g., every uridine is modified to a 1-methyl-pseudouridine). Exemplary RNA modifications can be found in the RNA modification database (See, mods(dot)rna(dot)albany(dot)edu/home).

In some embodiments, the at least one chemical modification comprises a modified uridine residue. Exemplary modified uridine residues include, but are not limited to, pseudouridine, 1-methylpseudouridine, 1-ethylpseudouridine, 2-thiouridine, 4′-thiouridine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-0-methyl uridine.

In some embodiments, the at least one chemical modification comprises a modified cytosine residue. Exemplary nucleosides having a modified cytosine include 5-aza-cytidine, 6-aza-cytidine, pseudoisocytidine, 3-methyl-cytidine, N4-acetyl-cytidine, 5-formyl-cytidine, N4-methyl-cytidine, 5-methyl-cytidine, 5-halo-cytidine, 5-hydroxymethyl-cytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine, α-thio-cytidine, 2′-O-methyl-cytidine, 5,2′-O-dimethyl-cytidine, N4-acetyl-2′-O-methyl-cytidine, N4,2′-O-dimethyl-cytidine, 5-formyl-2′-O-methyl-cytidine, N4,N4,2′-O-trimethyl-cytidine, 1-thio-cytidine, 2′-F-aracytidine, 2′-F-cytidine, and 2′-OH-aracytidine.

In some embodiments, the at least one chemical modification comprises a modified adenine residue. Exemplary nucleosides having a modified adenine include 2-amino-purine, 2,6-diaminopurine, 2-amino-6-halo-purine, 6-halo-purine, 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine, 2-methyl-adenine, N6-methyl-adenosine, 2-methylthio-N6-methyl-adenosine, N6-isopentenyl-adenosine, 2-methylthio-N6-isopentenyl-adenosine, N6-(cis-hydroxyisopentenyl) adenosine, 2-methylthio-N6-(cis-hydroxyisopentenyl) adenosine, N6-glycinylcarbamoyl-adenosine, N6-threonylcarbamoyl-adenosine, N6-methyl-N6-threonylcarbamoyl-adenosine, 2-methylthio-N6-threonylcarbamoyl-adenosine, N6,N6-dimethyl-adenosine, N6-hydroxynoryalylcarbamoyl-adenosine, 2-methylthio-N6-hydroxynoryalylcarbamoyl-adenosine, N6-acetyl-adenosine, 7-methyl-adenine, 2-methylthio-adenine, 2-methoxy-adenine, α-thio-adenosine, 2′-O-methyl-adenosine, N6,2′-O-dimethyl-adenosine, N6,N6,2′-O-trimethyl-adenosine, 1,2′-O-dimethyl-adenosine, 2′-O-ribosyladenosine (phosphate), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the at least one chemical modification comprises a modified guanine residue. Exemplary nucleosides having a modified guanine include inosine, 1-methyl-inosine, wyosine, methylwyosine, 4-demethyl-wyosine, isowyosine, wybutosine, peroxywybutosine, hydroxywybutosine, undermodified hydroxywybutosine, 7-deaza-guanosine, queuosine, epoxyqueuosine, galactosyl-queuosine, mannosyl-queuosine, 7-cyano-7-deaza-guanosine, 7-aminomethyl-7-deaza-guanosine, archaeosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methyl-inosine, 6-methoxy-guanosine, 1-methyl-guanosine, N2-methyl-guanosine, N2,N2-dimethyl-guanosine, N2,7-dimethyl-guanosine, N2,N2,7-dimethyl-guanosine, 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine, N2-methyl-2′-O-methyl-guanosine, N2,N2-dimethyl-2′-O-methyl-guanosine, 1-methyl-2′-O-methyl-guanosinc, N2,7-dimethyl-2′-O-methyl-guanosinc, 2′-O-methyl-inosine, 1,2′-O-dimethyl-inosine, and 2′-O-ribosylguanosine (phosphate).

In certain embodiments, the nucleic acid sequences encoding the fusion proteins described herein are present in lipid nanoparticles (e.g., for intravenous delivery to a human). Lipid nanoparticle compositions may include one or more cationic and/or ionizable lipids, phospholipids, neutral or non-cationic lipids, polyethylene glycol (PEG)-lipid conjugates, and/or sterols. In some embodiments, the lipid nanoparticle comprises a cationic lipid and/or ionizable lipid, a neutral or non-cationic lipid, and cholesterol. Cationic and/or ionizable lipids include, for example, amine-containing lipids that can be readily protonated and may have a positive or partial positive charge at physiological pH due to a pKa value between pH 5 and 8. The polar headgroup of the cationic lipids preferably comprises amine derivatives such as primary, secondary, and/or tertiary amines, quaternary ammonium, various combinations of amines, amidinium salts, or guanidine and/or imidazole groups as well as pyridinium, piperazine and amino acid headgroups such as lysine, arginine, ornithine and/or tryptophan. Cationic lipids include, but are not limited to, 1,2-dimyristoyl-sn-glycero-3-ethylphosphocholine (DMEPC), 1,2-di-O-octadecenyl-3-trimethylammonium propane (DOTMA) and/or 1,2-dioleoyl-3-trimethylammonium propane (DOTAP), 1,2-dimyristoyl-3-trimethylammonium propane (DMTAP), 2,3-di(tetradecoxy) propyl-(2-hydroxyethyl)-dimethylazanium bromide (DMRIE), didodecyl (dimethyl) ammonium bromide (DDAB), 1,2-dioleyloxypropyl-3-dimethyl-hydroxyethyl ammonium bromide (DORIE), 3β-[N-(N\N′-dimethylamino-ethane) carbamoyl]cholesterol (DC-Chol) or dioleyl ether phosphatidylcholine (DOEPC). Ionizable lipids include, but are not limited to, 1,2-dioleyloxy-3-dimethylamino-propane (DODMA).

In some embodiments, the lipid nanoparticle comprises a polyethylene glycol (PEG)-lipid conjugate. A PEG-lipid conjugate may include, but is not limited to, PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-DMG (1,2-dimyristoyl-rac-glycero-3-methoxypolyethylene glycol), PEG-c-DOMG (R-3-[(ω-methoxy poly(ethylene glycol) 2000) carbamoyl)]-1,2-dimyristyloxlpropyl-3-amine), PEG-DMA (PEG-dimethacrylate), PEG-DLPE (1,2-didodecanoyl-sn-glycero-3-phosphoethanolamine-PEG), PEG-DMPE (PEG-1,2-dimyristoyl-sn-glycero-3-phosphoethanolamine), PEG-DPPC (PEG-dipalmitoyl phosphatidylcholine), PEG-N,N-di (tetradecyl) acetamide, or a PEG-DSPE (1,2-distearoyl-sn-glycero-3-phosphocthanolaminc-poly(ethylene glycol)) lipid. In some embodiments, the lipid nanoparticle comprises PEG-DMG and/or PEG-N,N-di (tetradecyl) acetamide.

The sterol may comprise cholesterol, fecosterol, ergosterol, campesterol, sitosterol, stigmasterol, brassicasterol or a sterol ester, such as cholesteryl hemisuccinate, cholesteryl sulfate, or any other derivatives of cholesterol. A neutral or non-cationic lipid may include one or more phospholipids. Phospholipids include a phospholipid moiety and one or more fatty acid moieties. A phospholipid moiety may include, but is not limited to, phosphatidyl choline, phosphatidyl ethanolamine, phosphatidyl glycerol, phosphatidyl serine, phosphatidic acid, 2-lysophosphatidyl choline, and sphingomyelin. A fatty acid moiety may include, but is not limited to, lauric acid, myristic acid, myristoleic acid, palmitic acid, palmitoleic acid, stearic acid, oleic acid, linoleic acid, alpha-linolenic acid, erucic acid, phytanic acid, arachidic acid, arachidonic acid, eicosapentaenoic acid, behenic acid, docosapentaenoic acid, and docosahexaenoic acid.

Phospholipids suitable for use in the compositions may include, but are not limited to, phosphatidylglycerol (PG) including dimyristoyl phosphatidylglycerol (DMPG) and 1,2-dioleoyl-sn-glycero-3-phospho-rac-(1-glycerol) sodium salt (DOPG); phosphatidylcholine (PC), including egg yolk phosphatidylcholine, dimyristoyl phosphatidylcholine (DMPC), 1,2-distearoyl-sn-glycero-3-phosphocholine (DSPC), 1,2-dilinoleoyl-sn-glycero-3-phosphocholine (DLPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-didocosahexaenoyl-sn-glycero-3-phosphocholine, 1,2-diarachidonoyl-sn-glycero-3-phosphocholine; phosphatidylethanolamine (PE) including 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine; phosphatidic acid (PA); phosphatidylinositol (PI); phosphatidylserine (PS); and sphingomyelin (SM).

The positively charged lipid structures described herein may also include other components typically used in the formation of vesicles (e.g., for stabilization). Examples of such other components includes, without being limited thereto, fatty alcohols, fatty acids, and/or any other pharmaceutically acceptable excipients which may affect the surface charge, the membrane fluidity and assist in the incorporation of the lipid into the lipid assembly.

### EXAMPLE

### Example 1

### Generating SFTSV Gn-Ferritin Protein Nanoparticles

This example describes procedures used to generate Gn-Ferritin protein nanoparticles, which then could be used for immunization (e.g., with an adjuvant). The materials and methods to purify SFTSV Gn-ferritin protein nanoparticles were generally as described in Kim et al. (March/April 2021 Volume 12 Issue 2 e00230-21, which is herein incorporated by reference in its entirety, particularly for methods of generating antigen bearing Ferritin nanoparticles and methods of immunizing a test animal).

### Expression Vector Construction

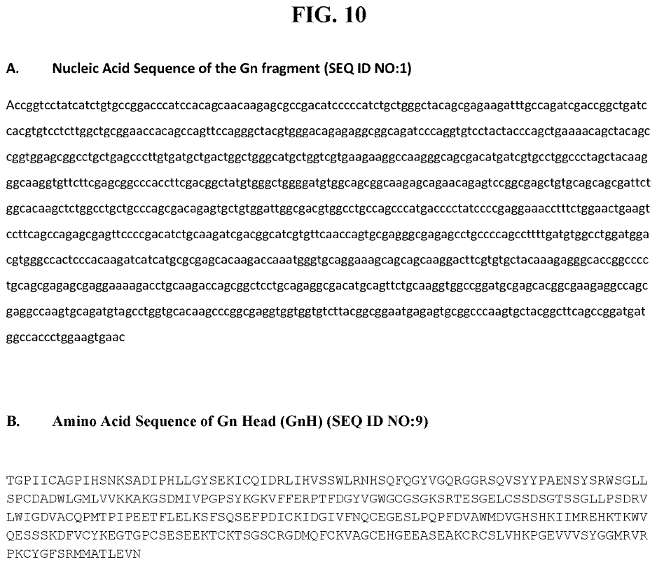

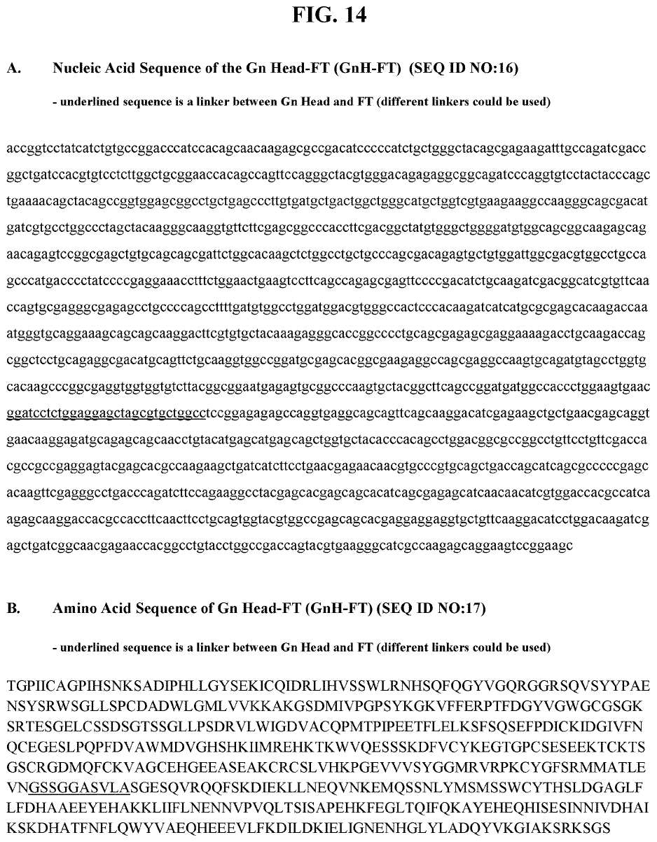

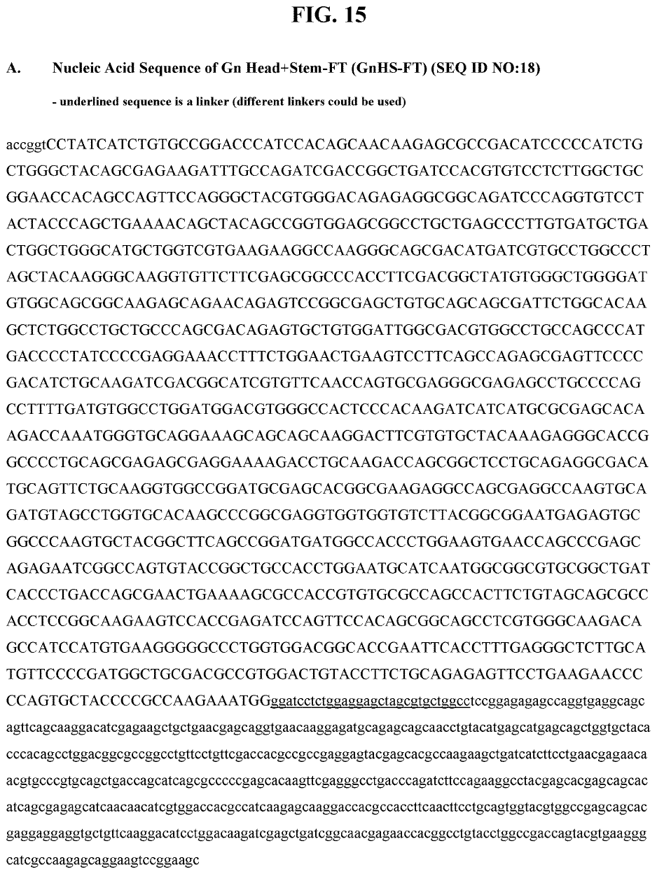

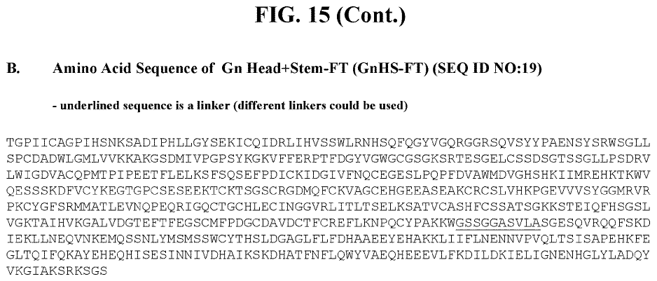

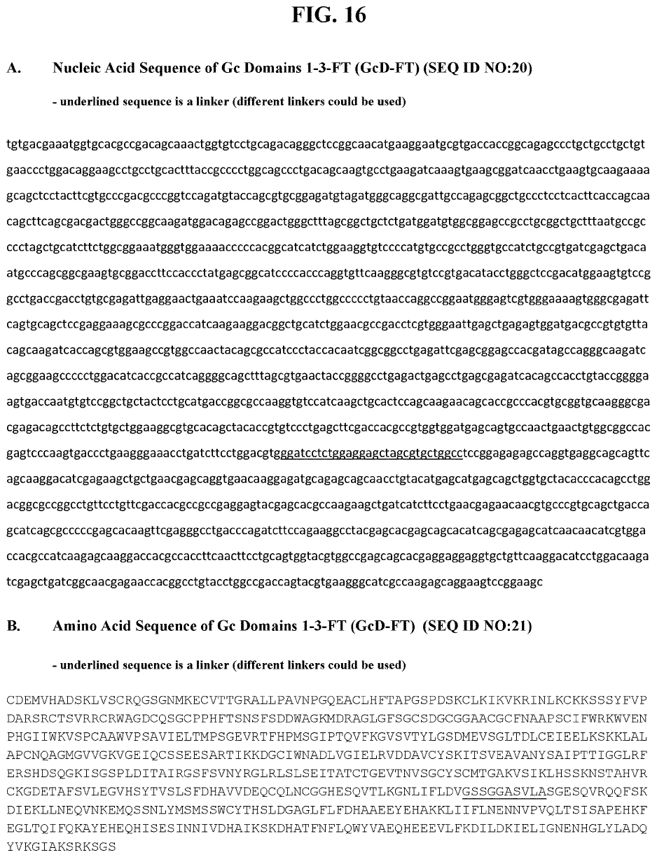

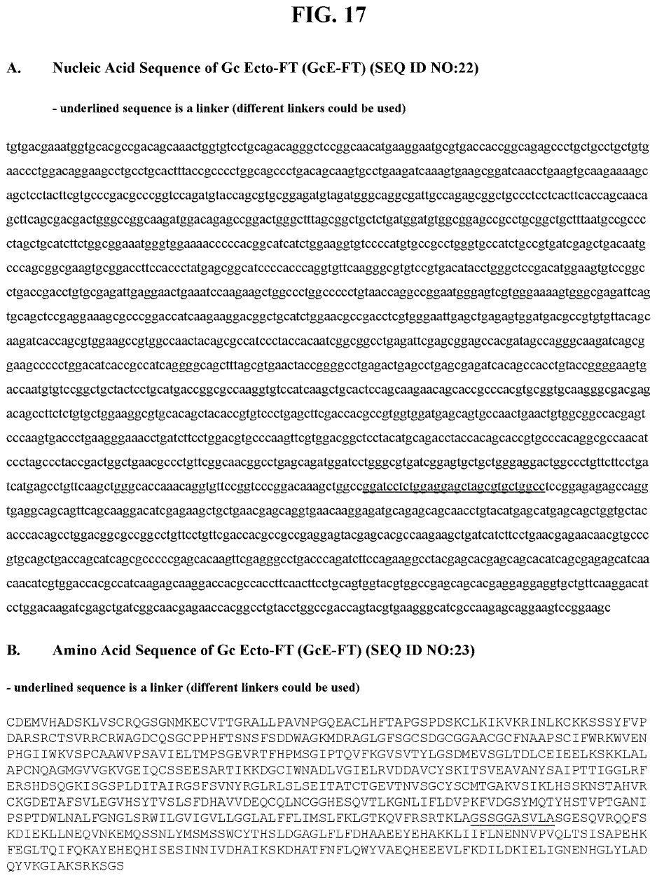

The gene encoding the recombinant ferritin engineered from Helicobacter pylori nonheme ferritin and the 2nd to 9th residues of the bullfrog (Rana catesbeiana) ferritin lower subunit was employed (SEQ ID NO:7), (See, Kanekiyo, M. et al. Cell, 162, and U.S. Pat. No. 10,744,199 to Kanekiyo et al., both of which are herein incorporated by reference. The gene encoding SFTSV Gn (Hubei strain) was codon optimized for human codon usage and synthesized by Genscript (New Jersey, United States) and is shown in FIG. 10 (SEQ ID NO:1). The gene was used to generate a fragment encoding the SFTSV Gn-SSGGASVLA linker-recombinant ferritin (shown in FIG. 14A, SEQ ID NO:16). For the expression plasmid, a commercially available pFUSE vector (Invivogen. California, United States) was engineered to replace the human ferritin light-chain gene promoter with the simian virus 40 (SV40) promoter. Genes encoding the recombinant ferritin and the SFTSV Gn-linker-ferritin fragment were cloned into the plasmid vector. The leader sequence used for expression was ATGGAAATCAAGG TGCTGTTTGCCCTCATCTGTATTGCTGTTGCTGAGGCA (SEQ ID NO:28).

### Expression and Purification of Nanoparticles:

HEK293T cells were purchased from the American Type Culture Collection (ATCC, Virginia, United States) and maintained in Dulbecco's modified Eagle's medium (DMEM) supplemented with 10% fetal bovine serum (FBS; Gibco, Massachusetts, United States) and 1% penicillin/streptomycin (Gibco). The cells were transiently transfected with polyethylenimine (Polysciences, Pennsylvania, United States) and respective vector plasmids in Opti-MEM and FreeStyle 293 medium (Gibco) supplemented with 3 mM valproic acid. Supernatants containing the nanoparticle were harvested 72 hours after transfection and initially purified by anion exchange chromatography method using Resource Q column (Cytiva lifesciences, Massachusetts, United States) on NGC fast protein liquid chromatography (Bio-Rad). Running buffer had gradient increase of NaCl concentration from 0 mM to 1 mM in phosphate buffered saline (PBS). Fractions eluted at 300 mM NaCl were pooled and further purified using size exclusion chromatography with Superdex 200 10/300 GL and Superose 6 PG (Cytiva,) on NGC and PBS as running buffer. Fractions at 0.45 column volume (CV) from size exclusion chromatography with Superdex 200 10/300 GL were further analyzed for purity of eFT (empty ferritin nanoparticles), and fractions at 0.5 CV from size exclusion chromatography with Superose 6 PG were further analyzed for the purity of sGnH-FT. The analyses for purity were performed by Coomassie blue staining and Western blot using in-house monoclonal mouse antibody recognizing SFTSV Gn.

### Successful Generation of Antibodies in Mice

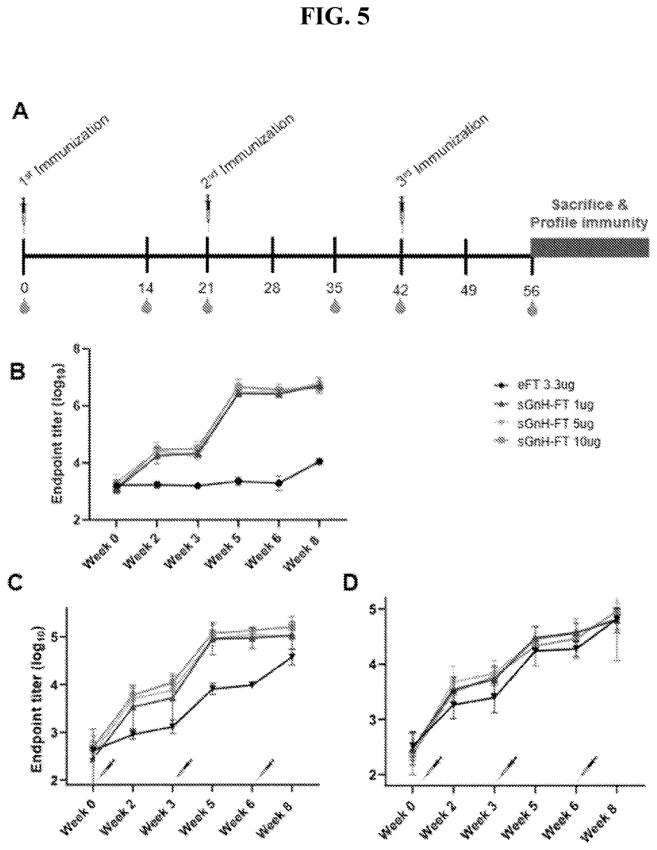

All animals were purchased from Jackson Laboratory (Maine, United States) housed at Lerner Research Institute Biological Resource Unit (BRU) accordingly to the approved animal experiment protocol. Female Balb/C mice of age from 6 to 8 weeks old were intramuscularly immunized in hind leg with 1 μg, 5 μg, or 10 μg of sGnH-FT or 3.3 μg of eFT (equivalent to 10 μg of sGnH-FT). Immunizations were performed at weeks 0, 3, and 6 (see FIG. 5A). All immunizations included AddaVax adjuvant (Invivogen) and matched to the final volume of 50 ul per immunization by adding PBS. Blood was collected from the immunized animals at weeks 0, 2, 3, 5, 6 and 8 to test for antibody development by retro-orbital bleeding.

FIG. 5B shows endpoint titration result against SFTSV GnH-10His antigen coated on ELISA plate. The antibody titer values were measured directly against the major viral antigen—SFTSV GnH—and the results demonstrate that 1 μg, 5 μg or 10 μg do not have significant difference in antibody titer generated from each immunization dose. On contrary, animals immunized with eFT did not develop any significant antibody titer against SFTSV GnH, with minimal induction only at the last blood drawing.

Specific antibody titer against sGnH-FT and eFT are demonstrated in FIGS. 5C and 5D. Endpoint titration from FIG. 5C used sGnH-FT, the vaccine candidate, as antigen to coat ELISA plates. Therefore, the titer demonstrated in 5C shows antibody recognizing both sGnH and the ferritin nanoparticle. Endpoint titration from FIG. 5D used eFT as antigen to coat ELISA plates.

Therefore, the titer demonstrated in 5D shows antibody recognizing only the carrier portion of the vaccine.

### Example 2

### SFTSV Gn-Ferritin Protein Nanoparticles Protect Against SFTSV Challenge

This example describes procedures testing a protein subunit vaccine with 24-mer self-assembling ferritin (FT) nanoparticle to present the SFTSV Gn head region (GnH) to enhance immunogenicity. Anion exchange chromatography and size exclusion chromatography readily purified the GnH-FT nanoparticles to homogeneity with structural integrity. Mice immunized with GnH-FT nanoparticles induced robust NAb response and T-cell immunity against DBV Gn. Furthermore, aged ferrets immunized with GnH-FT nanoparticles were fully protected from DBV challenge without SFTS symptoms such as body weight loss, thrombocytopenia, leukopenia, and fatality. This example demonstrates that DBV GnH-FT nanoparticles provide an efficient vaccine which could be administered to those with fatal SFTSV (aka DBV) infection.

### Results

### Molecular Design, Purification, and Characterization of DBV GnH-Ferritin Nanoparticles

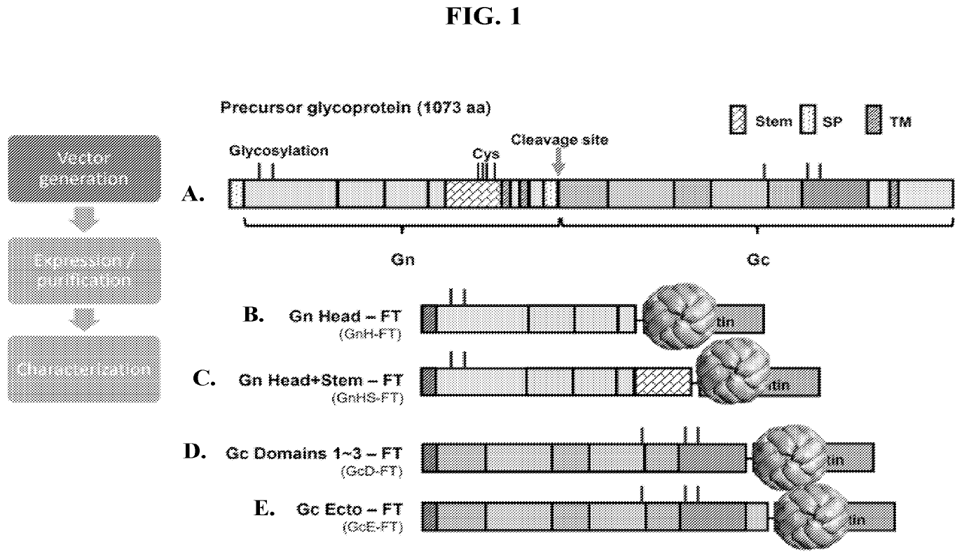

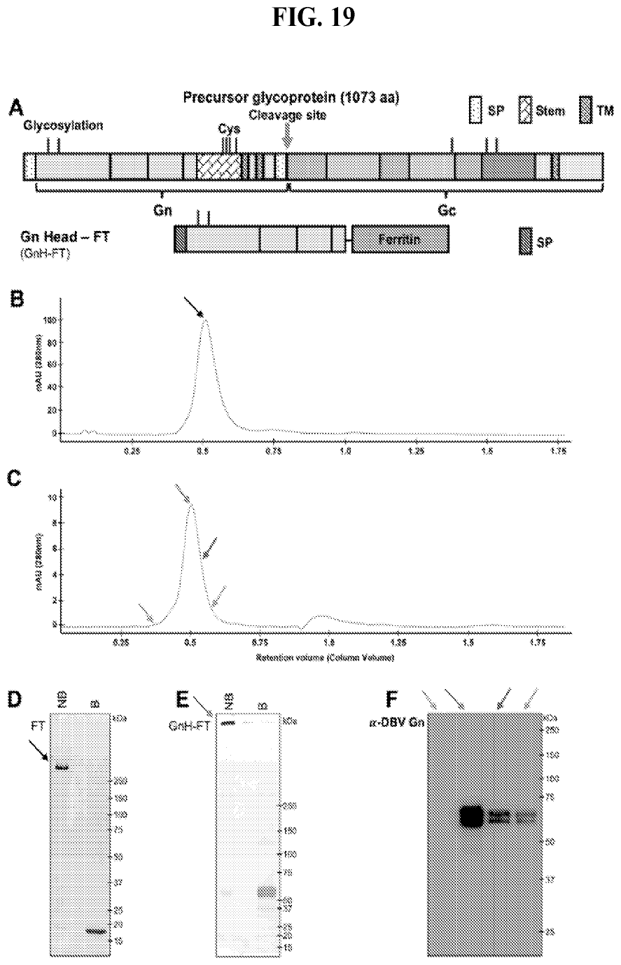

A majority of DBV-neutralizing antibodies from human convalescent sera target the head region of Gn (13, 14), which mediates viral attachment to host cells. Furthermore, our recent study of DBV DNA vaccine has identified the viral glycoproteins as the most immunogenic antigen among the viral proteins. The DBV Gn head (GnH) gene was first human-codon-optimized and fused to the IL-2 signal peptide at the N-terminus and the H. pylori-bullfrog hybrid ferritin at the C-terminus to generate GnH-ferritin (GnH-FT) (FIG. 19A). HEK293T cells were transfected with GnH-FT expression plasmid or the hybrid ferritin (FT) with the signal peptide, and the cell supernatants were collected to purify the GnH-FT and FT nanoparticles by anion exchange chromatography and size exclusion chromatography. The purified FT (FIG. 19B) and GnH-FT (FIG. 19C) nanoparticles were homogenous, as demonstrated by size exclusion chromatography with specific columns exhibiting maximal separating resolution at several hundred kilodaltons and a few megadaltons, respectively. Additionally, the chromatograms of GnH-FT and FT nanoparticles showed peaks at fractions corresponding to the expected molecular weight as 24-mer nanoparticles. We further tested if the purified FT and GnH-FT nanoparticles retained the 24-mer nanoparticle structures by loading the purified fractions onto SDS-PAGE without boiling (“NB” for not boiled) or with boiling (“B” for boiled). The purified nanoparticles retained the higher-order structure without boiling but disassembled into subunit monomers upon boiling (FIG. 19D, 19E). As a result, bands appeared at the expected molecular weights of monomers calculated based on previously solved GnH structures (15). Immunoblotting analysis of different fractions (marked with colored arrows) of size exclusion chromatography with anti-DBV Gn antibody also confirmed the presence of GnH-ferritin with intensities matching the peak heights of size exclusion chromatogram (FIG. 1F). These results indicate that the purified GnH-FT nanoparticles retain the higher-order structure from self-assembly.

### Purified GnH-FT Nanoparticle Retains the Higher-Order Structure and Presents GnH on its Surface

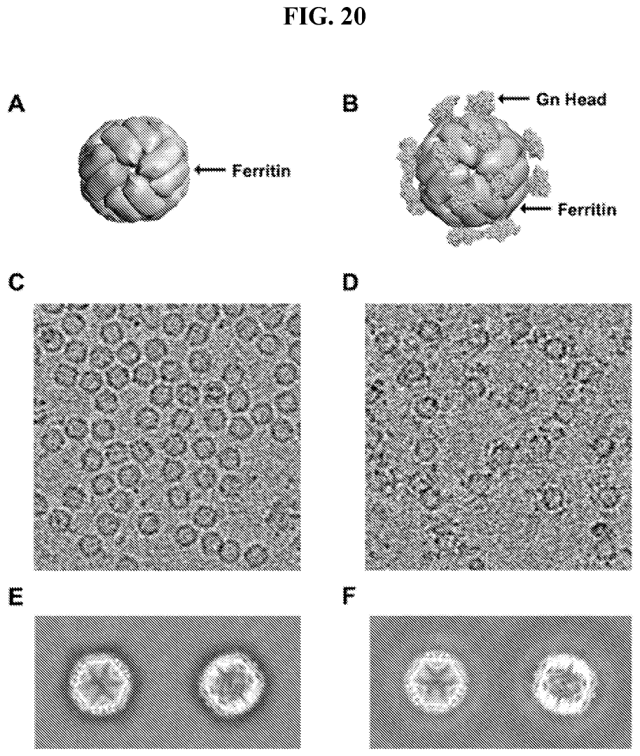

According to computer-assisted modeling based on previous reports using ferritin nanoparticles as carrier platforms, the GnH antigen was expected to radially project from three-fold axis points of the nanoparticle (FIG. 20A, 20B). Negative staining transmission electron microscopy (EM) and cryo-EM of FT nanoparticles demonstrated a homogenously smooth circular surface with an average diameter of 9.5 nm (FIG. 20C), while those of GnH-FT nanoparticles showed clearly visible protrusions from the ferritin core with an average diameter of 14.7 nm (FIG. 20D). These protrusions appeared as an extra layer of white, smeared halo surrounding the ferritin core in the cryo-EM 2D class averages of GnH-FT nanoparticles (FIG. 20F) compared to those of the FT nanoparticles (FIG. 20E). These data indicated that the GnH was flexible on the surface of the ferritin particle, and its presence did not affect the assembly of the ferritin particle.

Immunization with GnH-FT Nanoparticle Induces Humoral Immunity and Cellular Immunity In Vivo

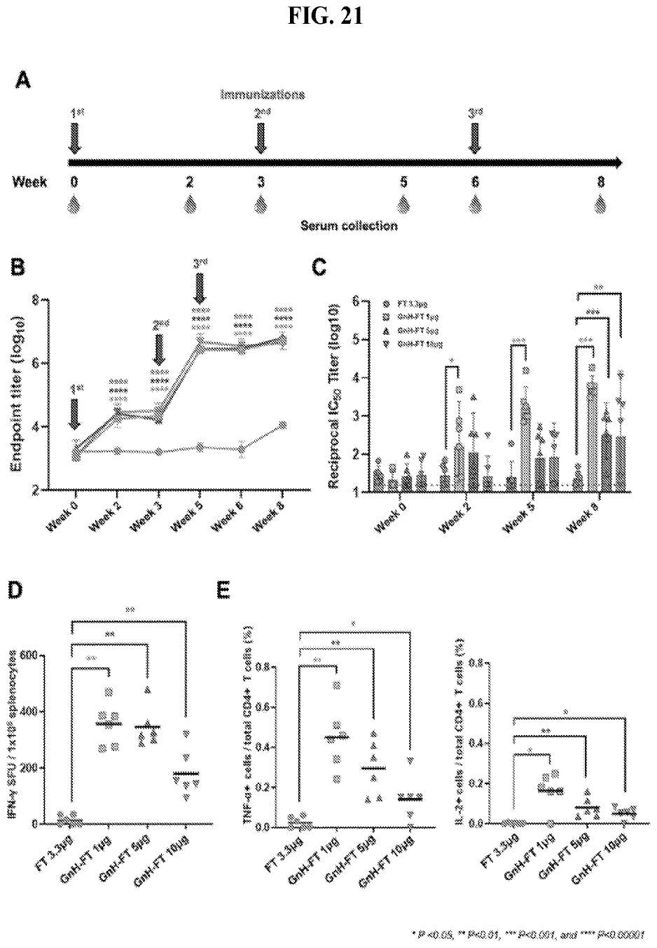

To enhance the relatively weaker immunogenicity of protein subunit vaccines, a diverse selection of adjuvants is administered in combination with protein vaccine candidate. One of the safest among the variety of adjuvants is MF59, an oil-in-water emulsion adjuvant used in adjuvanted flu vaccines (29-32). We combined the veterinary equivalent MF59, AddaVax, with FT or GnH-FT nanoparticles for immunization. 8-10-week-old BALB/c mice (n=6 per antigen) were immunized with a total of 3 doses at 3-week intervals of 3.3 μg of FT nanoparticle-200 equimolar to 10 μg of GnH-FT nanoparticle- or 1 μg, 5 μg, or 10 μg of GnH-FT nanoparticle via the intramuscular route. Blood was drawn prior to immunization (week 0) and every week starting 2 weeks after the priming immunization. Total IgG antibody against GnH soluble protein reached a maximal level at 2 weeks after the 2nd dose of immunization (first booster) and did not further increase upon the 3rd dose (second booster) among all three doses of GnH-FT nanoparticle immunization (FIG. 21B). Interestingly, there was no statistically significant difference in the induction of total anti-GnH IgG levels across different doses of GnH-FT nanoparticle, indicating that 1 μg GnH-FT dose was sufficient for inducing strong antibody response.

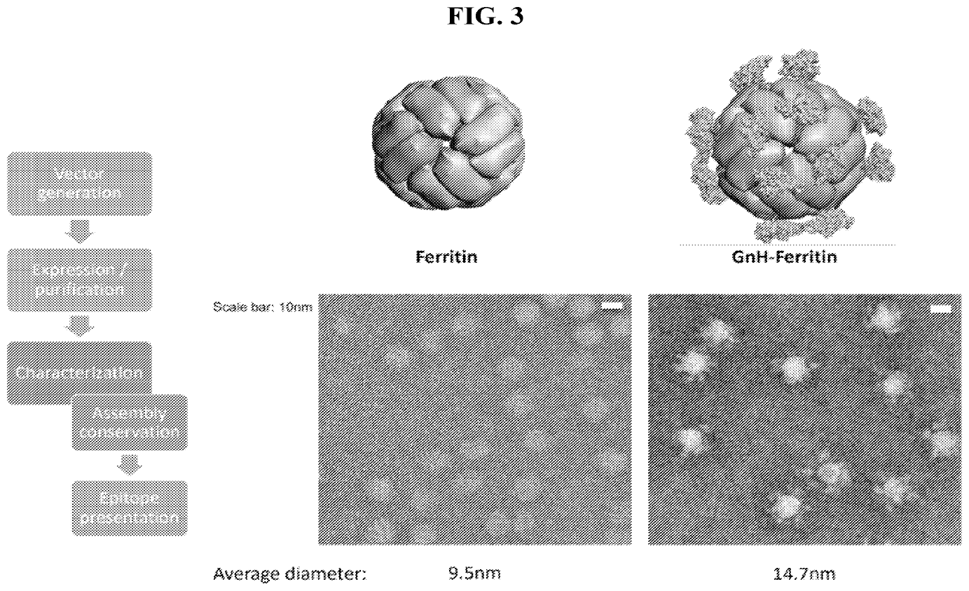

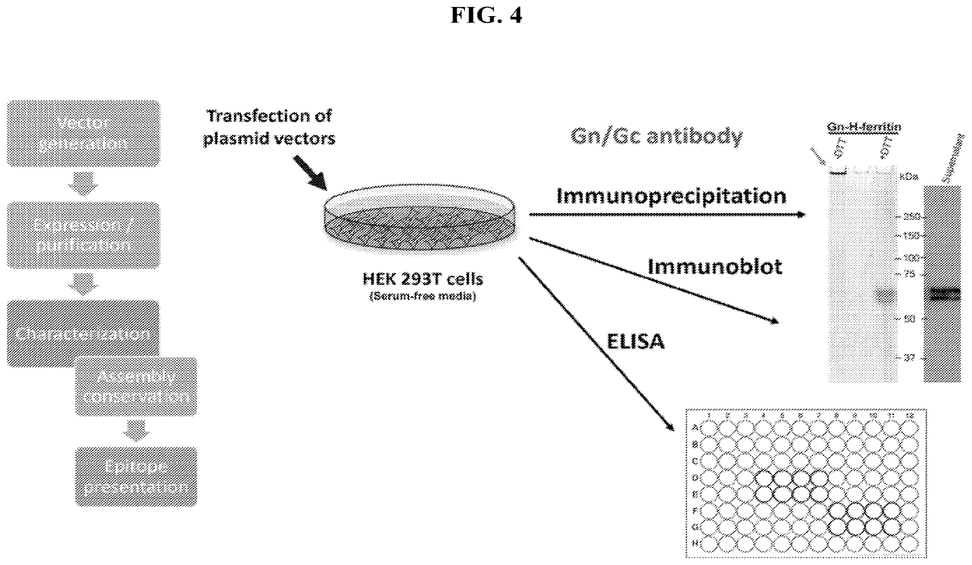

We performed a neutralization assay using replication-defective recombinant Vesicular Stomatitis Virus (rVSV) carrying DBV glycoproteins, Gn and Gc, and luciferase reporter gene (rVSV-DBV G). Unlike total anti-GnH antibody response that reached a maximal level after the 2nd immunization, neutralizing antibody (NAb) titer continuously increased over the 3 immunizations with GnH-FT nanoparticle (FIG. 21C). Immunization with 1 μg GnH-FT nanoparticle elicited the most robust neutralizing antibody response against DBV, followed by 5 μg and 10 μg. Consistently with the total IgG, there was no significant induction of NAb against DBV upon immunization with FT nanoparticle alone (FIG. 3C). These data suggest that 1 μg GnH-FT may be, in some embodiments, an optimal dose for 3-dose regimen to elicit strong NAb response against DBV infection.

Although NAb titer is a critical marker of vaccine efficacy, many studies have shown the importance of cellular immunity for antiviral immunity (33, 34). To perform IFN-γ ELISpot, spleens from immunized mice at week 8 (two weeks after the 3rd immunization) were harvested, ex vivo stimulated with a pool of overlapping peptides (OLPs) spanning GnH, and subsequently subjected to IFN-γ ELISpot. This showed that IFN-γ secretion was induced across all doses of GnH-FT nanoparticle immunization, whereas immunization with 1 μg GnH-FT nanoparticle elicited the most robust IFN-γ secretion (FIG. 21D). In addition, GnH-FT nanoparticle immunization successfully induced TNF-α and IL-2 production from OLP-stimulated CD4+ T cells, whereas FT-nanoparticle immunization did not induce those cytokine productions (FIG. 21E).

Finally, there was no significant production of TNF-α and IL-2 from OLP-stimulated CD8+ T cells (data not shown). These showed that the maximal induction of NAb, IFN-γ, TNF-α and IL-2 were observed from 1 μg dose of GnH-FT nanoparticle immunization. These data corresponded to the previous reports of robust activation of protective immunity from the low dose immunization of nanoparticle vaccines (25-27) and Adenoviral vector vaccine (35). Collectively, these results demonstrate that the immunization of DBV GnH-FT nanoparticle effectively elicits both NAb production and T-cell response in mice.

Aged Ferret Immunized with GnH-FT Nanoparticle Form Antibody Responses Against DBV

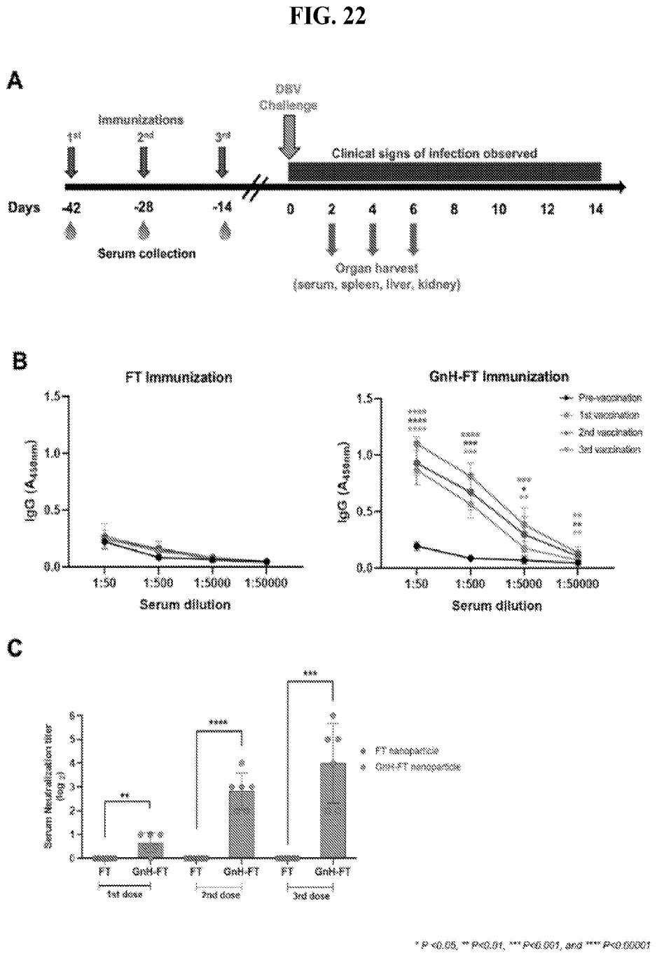

Naïve 4-year-old ferrets (n=12 ferrets per antigen) were vaccinated via intramuscular injection with FT or GnH-FT nanoparticles with Adda Vax adjuvant for a total of 3 immunizations at 2-week intervals. Blood of immunized ferrets was collected on the day of immunization to characterize antibody responses (FIG. 22A). While total anti-GnH IgG titers were dramatically increased after the 1st or 2nd vaccination, they were further escalated after the 3rd vaccination, suggesting that the vaccination protocol of total 3 doses maximizes the antibody response in aged ferret. We did not observe significant increase of IgG level upon booster immunizations, which might be due to the potential saturation of our assay (FIG. 22B). Consistently, serum NAb titers against DBV CB 1/2014 strain were continuously increased following the priming vaccination and subsequent booster vaccinations (FIG. 22C). On the contrary, neither anti-GnH antibody nor anti-DBV NAb was detected from control ferrets immunized with FT nanoparticles. These data indicate that the GnH-FT nanoparticle effectively induces antibody responses against DBV in aged ferret model.

Immunization with GnH-FT Nanoparticle Provides Full Protection Against Lethal DBV Challenge in Aged Ferrets

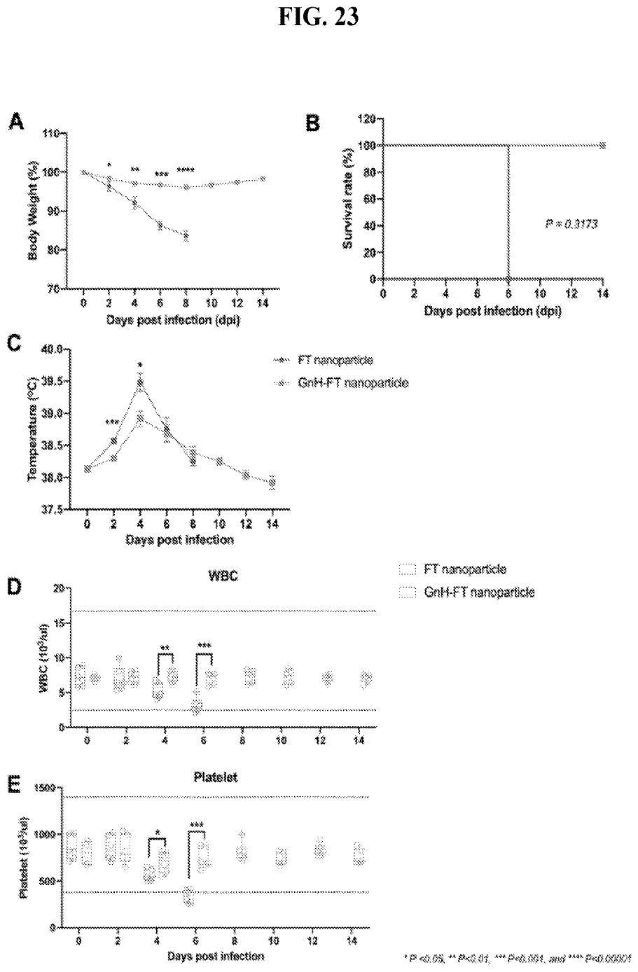

To evaluate the protective efficacy of GnH-FT vaccines, FT or GnH-FT vaccinated aged ferrets were intramuscularly challenged with a lethal dose of DBV CB1/2014 strain (107.6 252 TCID50) in 2 weeks after the third vaccination, and monitored for clinical signs of infection, viral titers and platelet counts in the blood, body weight, body temperature, and survival rate for the following 14 days, with evaluations every other day. Blood was collected every other day to measure platelet and white blood cell counts to observe thrombocytopenia and leukopenia (FIG. 22A). Strikingly, all ferrets immunized with GnH-FT nanoparticles were fully protected from lethal DBV challenge, while ferrets immunized with FT nanoparticles suffered significant body weight loss up to 20% and succumbed to death (FIGS. 23A and 23B). Aged ferrets immunized with GnH-FT nanoparticles showed minimal increases in body temperature, while those immunized with FT nanoparticles experienced severe fever (FIG. 23C). Platelet and white blood cell counts were also measured from the blood samples to test for the characteristic symptoms of thrombocytopenia and leukopenia. Consistent with body weight, temperature, and survival, aged ferrets immunized with GnH-FT nanoparticles showed little or no significant reduction in platelet and white blood cell counts (FIG. 23D). In contrast, aged ferrets immunized with FT nanoparticles demonstrated dramatic reductions in both platelet and white blood cell counts before the fatal outcome (FIG. 23E).

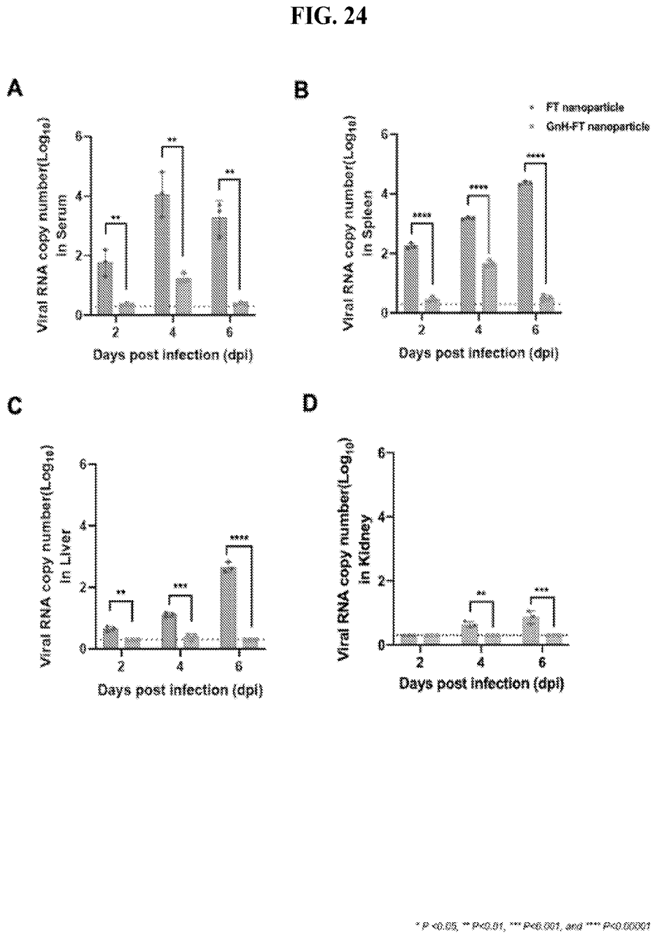

To assess viral burden in multiple organs upon lethal DBV challenge, we sacrificed 3 ferrets at days 2, 4, and 6 post-infection and harvested serum, spleen, liver, and kidney. Viral titers were measured as RNA copy numbers using real-time PCR. Aged ferrets immunized with FT-nanoparticle demonstrated significant viremia in liver and kidney upon lethal DBV challenge and succumbed to the viral challenge (FIGS. 6C and 6D). However, aged ferrets immunized with GnH-FT nanoparticles rapidly cleared the challenging virus so that the viral titer quickly decreased to the limit of detection or lower (FIGS. 24C and 24D). These data indicate that immunization with GnH-FT nanoparticles provides complete protection against SFTS pathogenesis upon lethal DBV challenge and promotes viral clearance in aged ferrets.

DBV, previously named SFTSV, is an emerging pathogen causing fatal SFTS in infected patients. Since its original discovery in China, it has established endemic infection in South Korea, Japan, and China, and spread to Southeast Asian countries (3, 4, 8). A clear age-dependence in pathogenesis from human DBV infection is evident, as the majority of hospitalization cases and almost all fatal infections occur in age groups of 50 or above (7). The vector tick—Haemaphysalis longicornis—used to have a relatively confined habitat in East Asia. However, its parthenogenetic reproduction has enabled recent rapid spread to other continents, including Australia and North America (36, 37). Combined with the spread of the tick, there have been growing concerns of a DBV outbreak beyond East Asia (1, 7, 37, 38). Here, we demonstrate the immunogenicity of the self-assembling GnH-FT nanoparticle as an effective DBV vaccine. Mice immunized with the FT nanoparticle vaccine induced potent antibody responses and cellular immunity. Immunized aged ferrets were fully protected from the lethal infection of DBV. Our results strongly demonstrate the DBV GnH-FT nanoparticle as a promising vaccine, providing protective immunity against DBV infection and subsequent SFTS pathogenesis.

Among DBV viral proteins, Gn and Gc glycoproteins are initially translated as a precursor glycoprotein from the M segment of the viral genome and subsequently processed into separate proteins by host protease (11). Structural analyses of closely related Bunyaviruses Heartland Bandavirus (HRTV) and Rift Valley Fever Virus (RVFV)—have identified that Gn and Gc form heterodimers and create higher-order structures on the viral surface (39, 40). Gn and Gc contribute to viral infection with Gn attaching to the host cell membrane and Gc mediating membrane fusion for endocytosis (12, 41). Our previous study demonstrated that the most potent immunogenicity was observed upon immunization of the Gn/Gc-containing M segment DNA in mice and aged ferrets (16).

Previous studies have demonstrated higher immunogenicity at lower doses of nanoparticle vaccines compared to conventional protein vaccines while retaining the advantage of reduced reactogenicity (26, 27). To develop a safe yet sufficiently immunogenic vaccine for aged population with weakened immunity, we formulated GnH-FT nanoparticle with an adjuvant with an established safety profile in the elderly population. Purified GnH-FT nanoparticles showed their integrity in presenting the DBV GnH on the carrier nanoparticle. Mice and aged ferrets immunized with GnH-FT nanoparticle robustly induced DBV-recognizing IgG and NAb.

While NAb titers increased over booster immunizations, the highest NAb titers were observed from the immunization with 1 μg of GnH-FT nanoparticle. Mice immunized with 1 μg GnH-FT nanoparticle also showed the strongest T cell response. This corresponds to the previous findings that immunizations with low doses of ferritin-fused nanoparticle provide protective immunity against Influenza virus (0.22 μg) (27), Epstein-Barr virus (0.5 μg) (26) and SARS-COV-2 vaccine (15 μg) (25). Similar results have been recently reported in a large clinical trial investigating the immunogenicity of Adenoviral vector (Ad26.COV2.S)-based vaccine against SARS-COV-2. Immunization with a lower dose (5E10) of Ad26.COV2.S particles induced more potent immunity with milder side effects than a higher dose (1E11) of Ad26.COV2.S particles (35). These studies further support the strong immunogenicity of ferritin nanoparticle at low doses as vaccine carrier. From our mouse in vivo studies, immunization with 1 μg GnH-FT provided the most robust induction of NAbs and T-cell responses. Because dose is closely related to vaccine-related adverse effects, additional studies are needed to optimize GnH-FT immunization protocols to enhance vaccine-mediated immunity while minimizing reactogenicity.

There are six genotypes of DBV (A to F) reported to date from South Korea, Japan, and China (7). Geographical locations with endemic DBV infection display varying abundances of these genotypes and, therefore, varying fatality rate. The GnH is encoded by the M segment of the DBV genome, which exhibits lower than 10% viral nucleotide variation (42, 43) and 6% amino acid variation (44), although the numbers vary across genotypes. Another study reported broad cross-reactivity of the HB29 strain of DBV (used in this study) with both autologous and hctcrologous genotypes of DBV (16, 45). Our previous DBV vaccine development also reported cross-protection of live-attenuated vaccines using HB29 (16, 46). This suggests that HB29 may serve as an optimal standard strain for DBV vaccine development. Protective immunity can be expanded even further for broader protection by exchanging the GnH on the FT nanoparticle with the GnH of other genotypes as “plug and play” platform.

### Material and Methods

### Expression and Purification of the Nanoparticles

Expression vectors to purify the nanoparticles were prepared as previously described in our earlier publication (25). Dabie Bandavirus (DBV) glycoprotein Gn gene (GenBank NC_018138.1) was codon-optimized for human codon usage (Genscript) and cloned into the expression vector. At 70% confluency, HEK293T cells (ATCC) cells had their media changed to FreeStyle 293 medium (Gibco) and transfected with the plasmids. Supernatants were concentrated with 100 kDa or 500 kDa MWCO filters on Labscale TFF (Sigma). Concentrated supernatants were flowed into anion Resource Q column (Cytiva) for anion exchange chromatography on NGC FPLC (Bio-Rad) running with pH8.0, 20 mM Tris-Cl and a gradient increase from 0 M to 1M NaCl at 3.0 ml/min. Fractions from NaCl concentration of 200 mM to 500 mM were collected and further purified by size exclusion chromatography. NGC FPLC equipped with Superdex 200 Increase 10/300 GL (for FT nanoparticle) and Superose 6 Increase 10/300 GL (for GnH-FT nanoparticle) columns (Cytiva) were used with PBS at 0.1 ml/min. Collected fractions were analyzed by loading onto SDS-PAGE with or without boiling for 10 min at 95 °C and stained with Coomassie Brilliant blue. Western blot of the fractions was performed using in house-generated mouse monoclonal antibody against DBV Gn.

To purify DBV GnH-10His protein, HEK293T cells were transfected with the mammalian expression vector under the same condition as for nanoparticle purification. Supernatant was flowed into HisTrap HP column (Cytiva) using NGC at flow rate of 5 ml/min and eluted by gradient increase of imidazole from 0 mM to 500 mM in 150 mM NaCl and 20 mM Tris-Cl, pH8.0. Fractions were tested for yield and purity by SDS-PAGE and stored at −80 °C in 10% glycerol.

### Computer-Assisted 3D Modeling of FT and GnH-FT Nanoparticles

Hypothetical structures of FT nanoparticle and GnH-FT nanoparticles were designed based on previously solved DBV Gn (PDB: 5Y11) and H. pylori-bullfrog hybrid ferritin (PDB: 3EGM) with Chimera (University of California San Francisco), PyMol (Schrodinger), and Meshmixer (Autodesk). The model was revised to take account for the size ratio of FT nanoparticles and GnH-FT nanoparticles.

Transmission Electron Microscopy and Cryo-EM Analysis of FT and GnH-FT Nanoparticles For negative staining transmission electron microscopy (EM), carbon-coated grids were rendered hydrophilic by glow-discharge and applied with a drop of purified nanoparticles in DPBS. After absorption for 1 min, excess sample was blotted away, and the grids were stained with 1% (w/v) uranyl acetate. After drying, the grids were imaged on a Talos F200X G2 microscope at 200 kV. To prepare cryo-EM grid, an aliquot of 3.5 μL purified nanoparticles at ~1 mg/mL concentration was applied to a 300-mesh Quantifoil R1.2/1.3 Cu grid pre-treated with glow-discharge, blotted in a Vitrobot Mark IV machine (force-5, time 3 s), and plunge-frozen in liquid ethane. The grid was loaded in a Titan Krios microscope equipped with Gatan BioQuantum K3 imaging filter and camera. A 20-eV slit was used for the filter. Data collection was done with serialEM (47). Images were recorded at 81,000× magnification, corresponding to a pixel size of 1.06 Å/pix. A defocus range of −1.0 μm to −1.8 μm was set. A total dose of 50 e-/Å2 of each exposure was fractionated into 50 frames. The first two frames of the movie stacks were not included in motion-correction. CryoEM data processing was performed on the fly with cryoSPARC Live (48) following regular single particle procedures.

### Virus Propagation and Titration

Methods from our previous publications were applied for DBV propagation and titration (16, 17). Briefly, Vero E6 (ATCC, CRL-1586) cells were cultured at 37 °C and 5% CO₂ with Dulbecco's modified Eagle's medium (DMEM) supplemented with 10% FBS. Cells were infected with CB1/2014 strain of DBV at confluency and supernatant was collected 7 days later. The supernatant was centrifuged to remove cell debris and stored at −80 °C until further use. Viral titer in form of TCID50 was determined by immunofluorescence assay (IFA) using in house-generated mouse monoclonal antibody recognizing DBV nucleoprotein (Np).

### Animal Care

BALB/c mice at ages of 6-8 weeks (Jackson Laboratories, Maine) were housed in Biological Resource Unit facility within Lerner Research Institute, Cleveland Clinic, with 12 h light/dark cycle with access to water and diet. Aged ferrets at ages of 48-50 months (ID Bio, Cheongju, South Korea) were housed in Laboratory Animal Research Center of Chungbuk National University (LARC) (Cheongju, South Korea) with 12 h light/dark cycle with access to water and diet. All mouse and ferret cares were performed in accordance with the institutional animal care guideline and experiment protocols approved by Institutional Biosafety Committee (IBC) and Institutional Animal Care and Use Committee (IACUC) in Cleveland Clinic and Chungbuk National University, respectively. After viral challenge, the animals were monitored more frequently by the authors or veterinary technicians on duty. Viruses were handled in an enhanced biosafety level 3 containment laboratory as approved by the Korean Centers for Disease Control and Prevention (KCDC-14-3-07).

### Animal Immunization and Sample Collection

Mice were intramuscularly immunized with 3.3 μg of FT nanoparticles, or 1 μg, 5 μg, or 10 μg of GnH-FT nanoparticles in hind leg. The antigens were prepared in 50 μl of DPBS and mixed with 50 μl AddaVax adjuvant (veterinary equivalent to MF59, Invivogen). Blood was collected from saphenous vein or retro orbital sinus to titer neutralizing antibodies. For ferret immunization, 15 μg FT nanoparticles or GnH-FT nanoparticles in 300 μl was mixed with 300 μl of AddaVax adjuvant for intramuscular immunization into the legs under anesthesia. Blood was also collected at the anesthesia. Subsequently, ferrets were intramuscularly infected with 107.6 TCID50/mL of DBV, which has shown 100% fatality in our previous study (46). Their body weight and temperature were measured, and veterinary clinical symptoms were observed. Blood was collected for hematological analysis every other day until 14 days post infection. Three animals per group were sacrificed at days 2, 4, and 6 to collect serum, spleen, liver, and kidney with individual scissors to avoid cross-contamination.

### Titration of DBV Gn-Recognizing Antibodies and DBV-Neutralizing Antibodies in Serum

To measure total mouse IgG against GnH, FT and GnH-FT, ELISA plates (MaxiSorp, ThermoFisher) were coated with respective antigens at concentration of 0.1 μg/well. The plates were blocked with 5% skim milk in 0.05% PBS-Tween 20. Heat-inactivated sera was diluted in 10-fold dilution series in DPBS and 100 μl of the dilutions were incubated in the wells overnight at 4 °C. Plates were washed and incubated with HRP-conjugated anti-mouse IgG antibody (Jackson Immunoresearch). For detection of antibodies, the plates were overlaid with TMB substrate (ThermoFisher) and 1M sulfuric acid.

To measure mouse neutralizing antibody titer against DBV, we performed pseudovirus neutralization assay as described in our previous publication (41). In brief, we co-incubated serially 2-fold diluted sera with recombinant VSV carrying DBV glycoproteins and reporter luciferase gene (rVSV-DBV-Luc). The inoculum was added to HEK293T cells and incubated at 37 °C with 5% CO₂ and the luciferase signal was with analyzed using luciferase assay kit (Promega).

For titration of total ferret IgG and neutralizing antibodies against DBV, we performed ELISA and serum neutralization titration as previously described (16, 46). To titer total ferret IgG titer, ELISA plates coated with antigen and blocked in same method as from mouse IgG titer. Ferret sera were diluted in 2% skim milk in 0.05% PBS-Tween 20 from 1:50 to 1:50,000. 100μ of diluted ferret sera were incubated in ELISA plates for 2 h at room temperature. Plates were then washed and incubated with HRP-conjugated anti-ferret IgG (KPL, South Korea). O-phenylenediamine dihydrochloride substrate was added to develop color and 1M sulfuric acid stop solution was added. After washing ELISA plates coated with GnH, the plates were blocked and incubated with HRP-conjugated anti-ferret IgG (KPL, South Korea). O-phenylenediamine dihydrochloride (ThermoFisher) was added to the plates and 1M sulfuric acid was added to stop color development. OD values at 450 nm were measured with a plate reader (iMark Microplate reader, Bio-Rad).

Serum neutralization titer was measured as previously described (49). Briefly, heat-inactivated serum samples were serially twofold diluted from a 1:2 to 1:128. Then, 50 μl of the diluted serum were mixed with equal volume of 200 focus-forming units of DBV for 1 h at 37 °C. The mixture was adsorbed onto confluent Vero E6 cells in 96-well plate at 37 °C for 1h. Media was changed to maintenance medium and cells were maintained in incubator for 5 days. Cells were then fixed with 10% formalin and stained with in house-generated anti-DBV Np antibody. For FRNT50, the cells were also stained with HRP-conjugated anti-mouse IgG antibody. Serum neutralizing antibody titer was presented as reciprocal of the highest serum dilution neutralizing fluorescence signal of DBV Np.

### Profiling T-Cell Immunity by IFN-γ ELISpot

We referred to our previous publication (16). In brief, Multiscreen 96-well plates with PVDF membrane (Milipore) were coated with 100 ul of anti-mouse IFN-γ antibody (clone AN-18, eBioscience, South Korea) overnight at 4 °C. Mouse splenocytes were stimulated ex vivo with overlapping peptide pool (OLP) of 78 of 15-mer peptides covering DBV GnH formulated at 0.625 μg/ml for each peptide in RPMI medium (Gibco) in the 96-well plate. PMA at 10 ng/ml and ionomycin at 500 ng/ml were included as positive controls and 0.5% DMSO was included as negative control for stimulation. After 24 h stimulation in 5% CO2, 37 °C incubator, plates were washed to remove cells and incubated with 100 μl of biotinylated anti-mouse IFN-γ antibody for 1 h at RT, followed by wash and incubation with 100 μL of streptavidin-alkaline phosphatase (Invitrogen) for 1 h at RT. 100 μl of BCIP/NBT was added for 10 min incubation at RT. Number of spot forming units (SFUs) per cells were presented after subtracting SFUs from negative control wells stimulated with 0.5% final concentration of DMSO.

### Intracellular Cytokine Staining

Based our protocol from previous publication (16), we resuspended the splenocytes from the immunized in 100 μl of RPMI-1640 media. The cells were ex vivo stimulated with anti-CD107a antibody (BD Biosciences, 553792), anti-CD28/CD49d antibody (BD Biosciences, 347690) and OLP or DMSO in 100 μl. The mixture was incubated in 5% CO2, 37 °C incubator 1 hour and treated with 4 μl of mixture of the complete RPMI-1640 media: Brefeldin A (GolgiPlug, BD Biosciences, 555029): Monensin (GolgiStop, BD Biosciences, 554715) in 55:3:2. After 12 hours of incubation in 5% CO2, 37 °C, the cells were washed with PBS and stained with surface antibodies (anti-CD44 BV421 [BD Biosciences, 536970], anti-CD8a BV510 [BD Biosciences, 563068], anti-CD62L BV650 [BD Biosciences, 564108], anti-CD3 BV786 [BD Biosciences, 564010], anti-CD4 PerCP-Cy5.5 [BD Biosciences, 561115], and anti-CD19 APC [BD Biosciences, 561738]) for 15 minutes at RT and washed. Cells were then fixed with 4% paraformaldehyde in PBS and permeabilized. We then washed the cells and stained with FACS antibodies (anti-IL-2 FITC [BD Biosciences, 562040], anti-TNF PE [BD Biosciences, 554419], anti-IFN-γ [BD Biosciences, 557735], and anti-CD107a [BD Biosciences, 560647]) by 20 minutes of incubation at room temperature, washed twice with the permeabilization buffer and resuspended with 300 μl of PBS.

Hematological Analysis and Viral Titration from Challenged Ferrets

We analyzed for hematological profile and tittered viremia as previously described (17). Total white blood cell and platelet counts in whole ferret blood samples were analyzed using the Celltac hematology analyzer (MEK-6550J/K, Nihon Kohden, Japan). Total RNA was extracted with TRIzol reagent (ThermoFisher) and reverse-transcribed to generate cDNA using QuantiTect Reverse Transcription system (Qiagen). Primers for real-time RT PCR (F: AATTCACATTTGAGGGTAGTT (SEQ ID NO:29), R: TATCCAAGGAGGATGACAATAAT (SEQ ID NO:30) were designed to recognize M segment of DBV genomc. Real-time PCR was performed with SYBR Green supermix and CFX Real-Time PCR detection system (Bio-Rad). Copy numbers were normalized to GAPDH gene.

### REFERENCES FOR EXAMPLE 2

1. Casel et al., Severe fever with thrombocytopenia syndrome virus: emerging novel phlebovirus and their control strategy. Experimental & Molecular Medicine 53:713-722.
2. Yu et al., 2011. Fever with thrombocytopenia associated with a novel bunyavirus in China. N Engl J Med 546 364:1523-32.
3. Rattanakomol et al., 2022. Severe Fever with Thrombocytopenia Syndrome Virus Infection, Thailand, 2019-2020. Emerg. Infect Dis 28:2572-2574.
4. Tran et al., 2019, Endemic Severe Fever with Thrombocytopenia Syndrome, Vietnam. Emerg Infect Dis 25:1029-552 1031.
5. Jiang et al., 2015. A cluster of person-to-person transmission cases caused by SFTS virus in Penglai, China. Clin Microbiol Infect 21:274-9.
6. Zhang et al., 2012. Hemorrhagic fever caused by a novel Bunyavirus in China: pathogenesis and correlates of fatal outcome. Clin Infect Dis 54:527-33.
7. Yun et al., 2020. Genetic and pathogenic diversity of severe fever with thrombocytopenia syndrome virus (SFTSV) in South Korea. JCI Insight 5.
8. Li et al., 2021. Severe fever with thrombocytopenia syndrome virus: a highly lethal bunyavirus. Crit Rev Microbiol 47:112-125.
9. Anonymous. 2018. Annual review of diseases prioritized under the Research and Development Blueprint, Informal consultation. World Health Organization, https://www.who.int/docs/default-source/blue-print/2018-annual-review-of-diseases-568 prioritized-under-the-research-and-development-blueprint.pdf?sfvrsn=4c22e36_2.
10. Diseases NIoAaI. 2018. NIAID Emerging Infectious Diseases/Pathogens-Category C. Diseases BaEI,
11. Plegge et al., 2016. Evidence that Processing of the Severe Fever with Thrombocytopenia Syndrome Virus Gn/Gc Polyprotein Is Critical for Viral Infectivity and Requires an Internal Gc Signal Peptide. PLOS One 11: c0166013.
12. Halldorsson et al., 2016. Structure of a phleboviral envelope glycoprotein reveals a consolidated model of membrane fusion. Proc Natl Acad Sci USA 113:7154-9.
13. Guo et al., 2013. Human antibody neutralizes severe Fever with thrombocytopenia syndrome virus, an emerging hemorrhagic Fever virus. Clin Vaccine Immunol 20:1426-32.
14. Kim et al., 2019. An anti-Gn glycoprotein antibody from a convalescent patient potently inhibits the infection of severe fever with thrombocytopenia syndrome virus. PLOS Pathog 15: e1007375.
15. Wu et al., 2017. Structures of phlebovirus glycoprotein Gn and identification of a neutralizing antibody epitope. Proc Natl Acad Sci USA 114: E7564-E7573.
16. Kwak et al., 2019. Development of a SFTSV DNA vaccine that confers complete protection against lethal infection in ferrets. Nature Communications 10:3836.
17. Park et al., 2019. Ferret animal model of severe fever with thrombocytopenia syndrome phlebovirus for human lethal infection and pathogenesis. Nat Microbiol 4:438-446.
18. Choi et al., 2020. Severe Fever with Thrombocytopenia Syndrome Virus NSs Interacts with TRIM21 To Activate the p62-Keap1-Nrf2 Pathway. J Virol 94.
19. Zimmermann P, Curtis N. 2019. Factors That Influence the Immune Response to Vaccination. Clinical Microbiology Reviews 32: e00084-18.
20. Pollard A J, Bijker E M. 2021. A guide to vaccinology: from basic principles to new developments, Nature Reviews Immunology 21:83-100.
21. Gause et al., 2017. Immunological Principles Guiding the Rational Design of Particles for Vaccine Delivery. ACS Nano 11:54-68.
22. Porter CJH, Trevaskis N L. 2020. Targeting immune cells within lymph nodes. Nature Nanotechnology 15:423-425.
23. Kelly et al., 2020. Self-assembling influenza nanoparticle vaccines drive extended germinal center activity and memory B cell maturation. JCI Insight 5.
24. Link et al., 2012. Innate immunity mediates follicular transport of particulate but not soluble protein antigen. J Immunol 188:3724-33.
25. Kim et al., 2021. Development of Spike Receptor-Binding Domain Nanoparticles as a Vaccine Candidate against SARS-COV-2 Infection in Ferrets. mBio 12.
26. Kanekiyo et al., 2015, Rational Design of an Epstein-Barr Virus Vaccine Targeting the Receptor-Binding Site. Cell, 162:1090-100.
27. Kanckiyo et al., 2013. Self-assembling influenza nanoparticle vaccines elicit broadly neutralizing H1N1 antibodies. Nature 499:102-6.
28. Kim et al., 2018. Chaperna-Mediated Assembly of Ferritin-Based Middle East Respiratory Syndrome-Coronavirus Nanoparticles. Front Immunol 9:1093.
29. Domnich et al., 2017. Effectiveness of MF59-adjuvanted seasonal influenza vaccine in the elderly: A systematic review and meta-analysis. Vaccine 35:513-520.
30. Podda A, Del Giudice G. 2003. MF59-adjuvanted vaccines: increased immunogenicity with an optimal safety profile. Expert Review of Vaccines 2:197-204.
31. Pellegrini et al., 2009. MF59-adjuvanted versus non-adjuvanted influenza vaccines: integrated analysis from a large safety database. Vaccine 27:6959-65.
32. Tsai T F. 2013. Fluad (R)-MF59 (R)-Adjuvanted Influenza Vaccine in Older Adults. Infect Chemother, 45:159-74.
33. Maini M K, Pallett L J. 2018. Defective T-cell immunity in hepatitis B virus infection: why therapeutic vaccination needs a helping hand. Lancet Gastroenterol Hepatol 3:192-202.
34. Thimme R. 2021. T cell immunity to hepatitis C virus: Lessons for a prophylactic vaccine. J Hepatol 74:220-229.
35. Sadoff et al., 2021. Interim Results of a Phase 1-2a Trial of Ad26.COV2.S Covid-19 Vaccine. New England Journal of Medicine 384:1824-1835.
36. (CDC) CfDCaP. 2023. What you need to know about Asian longhorned ticks-a new tick in the United States.
37. Zhang et al., 2022. Rapid Spread of Severe Fever with Thrombocytopenia, Syndrome Virus by Parthenogenetic Asian Longhorned Ticks. Emerg Infect Dis 28:363-372.
38. Yun et al., 2014. Severe fever with thrombocytopenia syndrome virus in ticks collected from humans, South Korea, 2013, Emerg Infect Dis 20:1358-61.
39. Kimura et al., 2021. Characterization of pseudotyped vesicular stomatitis virus bearing the heartland virus envelope glycoprotein, Virology 556:124-132.
40. Lam et al., 2013, Evolutionary and molecular analysis of the emergent severe fever with thrombocytopenia syndrome virus. Epidemics 5:1-10.
41. Xia et al., 2023, Glucosylceramide is essential for Heartland and Dabie bandavirus glycoprotein-induced membrane fusion. PLOS Pathogens 19: e1011232.
42. Yoshikawa et al., 2015. Phylogenetic and Geographic Relationships of Severe Fever With Thrombocytopenia Syndrome Virus in China, South Korea, and Japan. J Infect. Dis 212:889-98.
43. Li et al., 2021. Molecular evolution and genetic diversity analysis of SFTS virus based on next-generation sequencing. Biosafety and Health 3:105-115.
44. Yun et al., 2017. Molecular genomic characterization of tick- and human-derived severe fever with thrombocytopenia syndrome virus isolates from South Korea. PLOS Negl Trop Dis 11: e0005893.
45. Jia et al., 2017. Identification of a candidate standard strain of severe fever with thrombocytopenia syndrome virus for vaccine quality control in China using a cross-neutralization assay. Biologicals 46:92-98.
46. Kwang-Min et al., 2019. Cross-genotype protection of live-attenuated vaccine candidate for severe fever with thrombocytopenia syndrome virus in a ferret model. PNAS 116:26900-26908.
47. Mastronarde D N. 2005. Automated electron microscope tomography using robust prediction of specimen movements. J Struct Biol 152:36-51.
48. Punjani et al., 2017. cryoSPARC: algorithms for rapid unsupervised cryo-E M structure determination. Nat Methods 14:290-296.
49. Yu et al., 2018, Seroprevalence and genetic characterization of severe fever with thrombocytopenia syndrome virus in domestic g oats in South Korea. Ticks Tick Borne Dis 9:1202-1206.

### Example 3

SFTSV Gn Head-Ferritin Nanoparticle mRNA Vaccine Confers Efficient Protection Against Lethal Challenge

This example describes the immunogenicity of mRNA vaccines encoding SFTSV Gn-H and Gn-H-FT nanoparticles as SFTSV vaccine candidates. This example includes expressions of sGn-H, mRNA encapsulation into lipid nanoparticles (LNPs) for efficient delivery and immunization of mice to assess the induction of humoral immunity against SFTSV. Both sGn-H and sGn-H-FT mRNA vaccines elicited considerable levels of SFTSV-neutralizing antibodies (NAbs) that remained elevated for up to 12 weeks following booster. Furthermore, we challenged immunized mice with sGn-H or sGn-H-FT mRNA LNP vaccine candidate with a lethal dose of SFTSV to evaluate protection against SFTS symptoms, including body weight loss and fatality. The results show that SFTSV Gn-H and Gn-H-FT mRNA LNPs represent promising vaccine candidates capable of providing protection against lethal SFTSV infection.

### Materials and Methods

Molecular Design of mRNA Encoding SFTSV Gn-H and Gn-H-FT

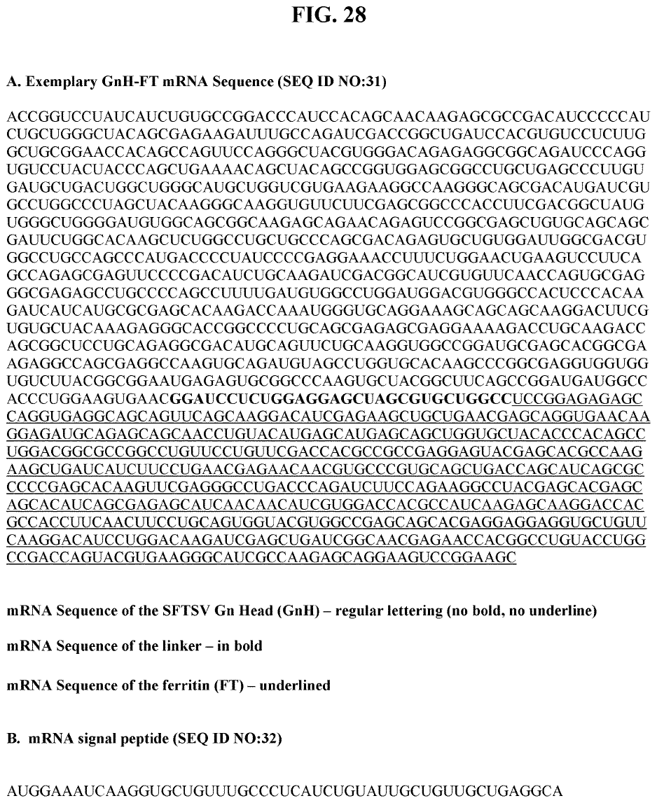

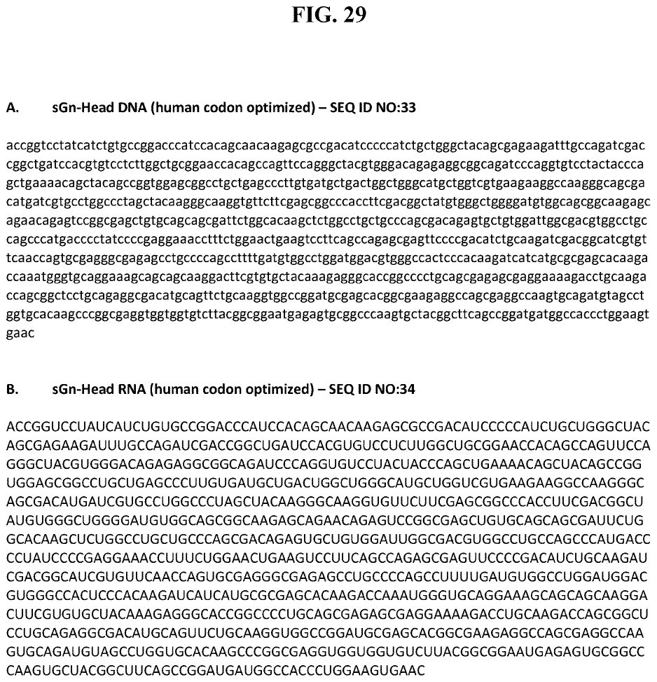

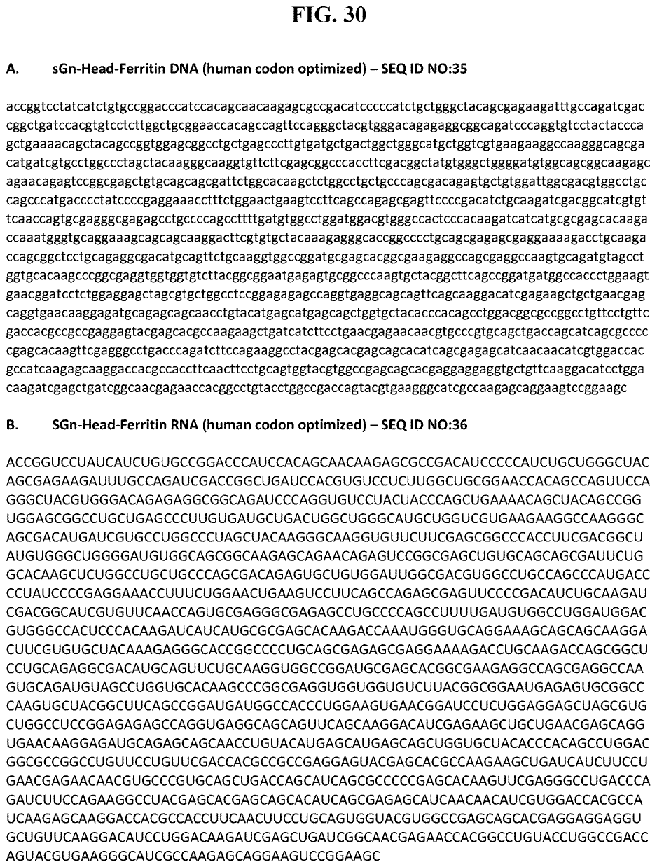

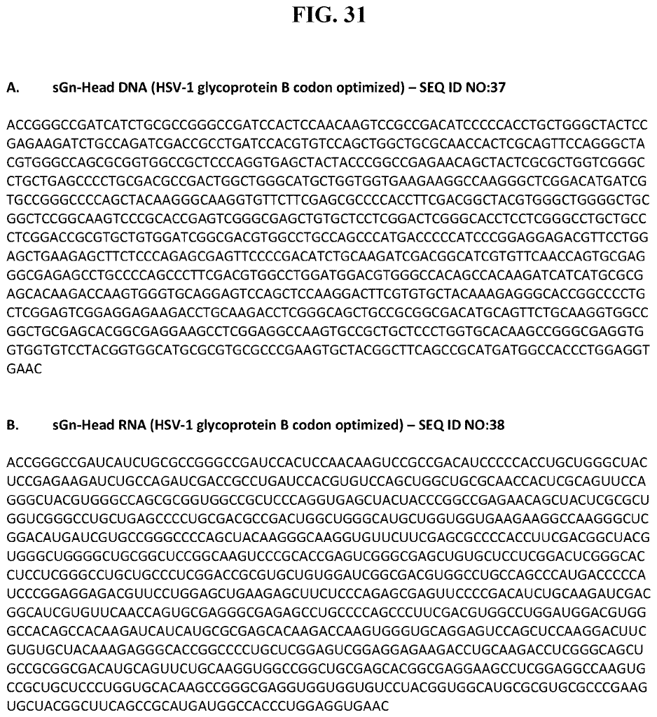

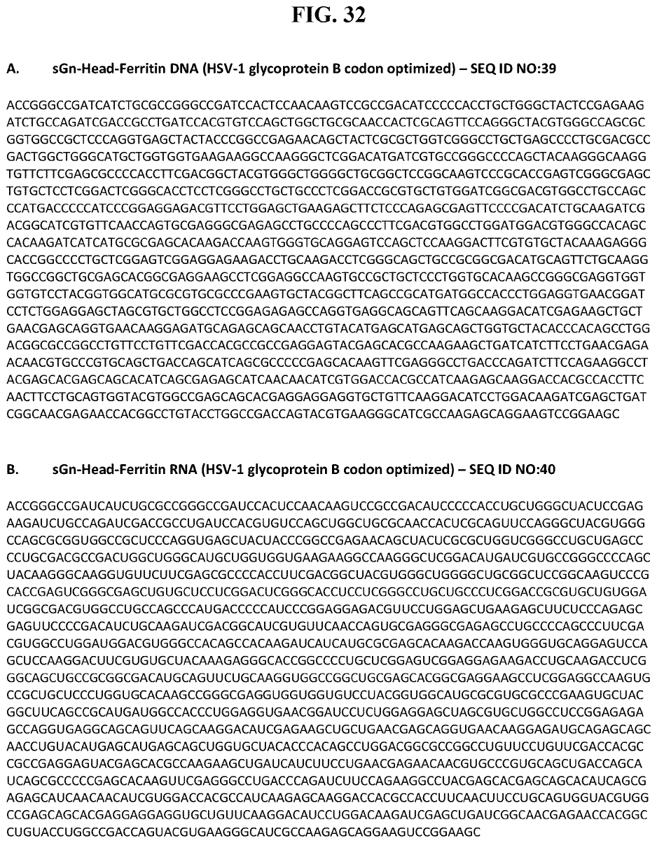

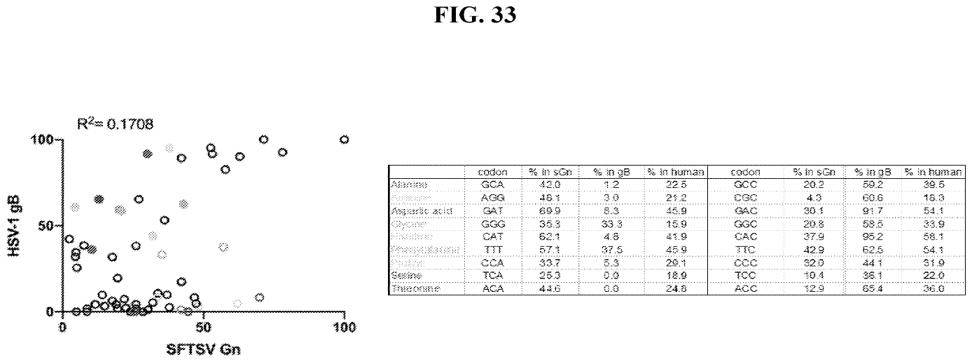

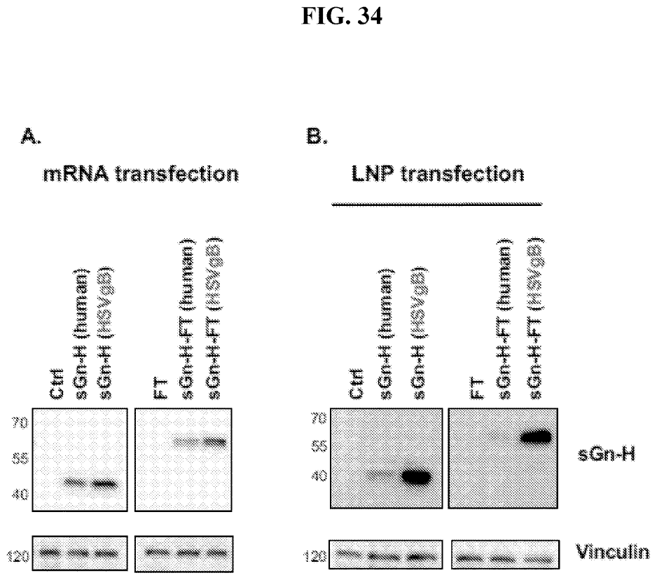

The GnH-FT construct was generated as previously described in our publication (26, herein incorporated by reference and is found at FIG. 28, SEQ ID NO:31). The head region of SFTSV was cloned into a vector engineered to incorporate the T7 promoter, as well as 5′ and 3′ UTRs (43). sGn-H was then fused with hybrid ferritin via a linker. The construct was also cloned into the same vector to make the sGn-H-FT construct. Linearized and purified DNA templates of sGn-H and sGn-H-FT, complete with the UTRs, underwent in vitro transcription (IVT) via T7 RNA polymerase (NEB). Co-transcriptional capping introduced N1-Methyl-PseudoUTP and CleanCap AG cap1 analog (TriLink), which (3′ OMe): N-7413-10. Enzymatic polyadenylation was accomplished using Poly-A polymerase (NEB). The resultant 5′-capped and polyadenylated mRNA products were purified via spin column (Zymo Research) and evaluated through RNA agarose gel electrophoresis.

DNA and mRNA Transfection to Characterize Expression

A mammalian expression vector was transfected into HEK293T cells. The DNA transfection was conducted utilizing PolyJet transfection reagent (SignaGen). The mRNA transfection was conducted at HEK293T with 1 μg of 5′-capped and polyadenylated linear mRNAs using TransIT-mRNA transfection reagent (Mirus). Supernatant and/or cell lysates were collected 24 hours post-transfection for subsequent analysis via SDS-PAGE and Western blot analysis.

mRNA Encapsulation into Lipid Nanoparticle (LNP)

The mRNA encapsulation process was carried out using a NanoAssemblr Ignite+microfluidic instrument (Precision nanosystems) at a ratio of 3:1 (aqueous mRNA: lipid in ethanol). The lipids used were Gen Voy-ILM (stands for ionizable lipid mix, catalog #NWW0042), which is described by the manufacturer as DSPC (1,2-distearoyl-sn-glycero-3-phosphocholine): cholesterol: ionizable lipid: stabilizer at ratio of 10:37.5:50:2.5. Subsequently, LNPs containing the mRNA (mRNA-LNPs) were diluted with DPBS (Gibco) and concentrated using Amicon centrifugal filters (Millipore) to eliminate ethanol. The resulting mRNA-LNP products were stored at 4 °C until immunization. Encapsulation efficiency and the mRNA concentration within the LNPs were verified using Ribogreen RNA assay (ThermoFisher) to ensure encapsulation efficiency surpassing 95%.

### Virus Propagation and Titration

Vero E6 cells (CRL-1586), confirmed negative for mycoplasma contamination, were procured from ATCC. Confluent Vero E6 cells were infected with the CB 1/2014 strain of SFTSV. The supernatants were harvested seven days post-infection and stored at −80 °C. Virus titer was determined by infecting Vero E6 cells with serial dilutions of the virus in overlay media consisting of DMEM supplemented with 3% FBS, 1% penicillin/streptomycin, 1.2% Avicel (FMC BioPolymer), 0.5% Glutamax, and 0.5% sodium pyruvate. The cells were maintained for 14 days, and the monolayers were fixed with 10% formaldehyde in PBS and stained with a solution containing 20% ethanol and 1% crystal violet for plaque counting.

Mouse Immunization and Infection with SFTSV

The studies were conducted in accordance with IBC (202 and FLIB002) and IACUC-approved protocols (2452 and 2503). BALB/c mice aged 6 to 8 weeks (Jackson laboratory) were intramuscularly immunized with LNP carrying 1 μg of sGn-H or sGn-H-FT mRNA at weeks 0 and 3. Blood samples were collected to obtain serum for subsequent immunological profiling. Type I Interferon receptor (Ifnar1) knockout mice (A129) of the same age (Jackson laboratory) received 3 μg of GnH or GnH-FT mRNA-LNPs at the same time points as the BALB/c mice. The mice were then transported to the Animal Biosafety Level-3 (ABSL-3) facility at the Florida Research and Innovation Center, where they remained until the conclusion of the study. For the challenge study, the mice were intramuscularly infected with a lethal dose of 2E4 plaque-forming units (pfu) of SFTSV. Clinical symptoms characterized as changes in body weight and survival were monitored over a period of 14 days.

### Titration of Overall IgG Antibodies Recognizing SFTSV GnH

The SFTSV Gn Head protein, purified in accordance with our previous literature (26), was coated onto MaxiSorp ELISA plates (ThermoFisher) at a concentration of 1 μg/ml. Subsequently, the plates were blocked overnight at 4 °C using 3% BSA in PBS-T. Serial dilutions of heat-inactivated sera in PBS were introduced into the wells, followed by an overnight incubation at 4 °C. After washing with PBS-T, the wells were treated with HRP-conjugated anti-mouse IgG antibody (ThermoFisher). Following another round of washing with PBS-T, the wells were added with TMB substrate (BioLegend) and 1 M sulfuric acid. Endpoint titer values were determined by calculating the dilution at which absorbance values exceeded twice the background absorbance values.

### Titration of SFTSV-Neutralizing Antibody by Pseudovirus Neutralization Assay

Recombinant Vesicular Stomatitis Virus (rVSV) pseudovirus, carrying SFTSV Glycoproteins and a luciferase reporter gene (rVSV-SFTSV G-Luc), was generated following procedures outlined in previous literature (26, 44). Heat-inactivated sera were serially diluted in PBS and mixed with rVSV-SFTSV G-Luc for a 1 hour incubation at 37 °C. The mixture was added to confluent HEK293T cells for infection for 1 hour, followed by washing and incubation at 5% CO₂ and 37 °C for 24 hours. Cells were lysed, and the luciferase signal was measured as relative light units (RLU) using luciferase assay system (Promega). Neutralization efficacy was normalized by defining RLU values from wells with no sera (cells infected with rVSV-SFTSV G-Luc without sera) as 100% neutralization and wells with no rVSV-SFTSV G-Luc (media only) as 0% neutralization. The neutralization titer was quantified as IC50 using a nonlinear function (log agonist vs. normalized response, variable slope) within GraphPad Prism.

### Statistical Analyses

Statistical analyses were performed using GraphPad Prism and Microsoft Excel. Mean values and error bars representing the standard deviation were presented for experiments conducted with three or more replicates. Geometric mean values with associated 95% confidence intervals were utilized to represent the levels of SFTSV GnH-recognizing antibodies and SFTSV-neutralizing antibodies. Group comparisons underwent statical analysis using a two-way analysis of variance (ANOVA), followed by a Student's t-test. Survival curves were statistically compared using the Mantel-Cox test.

### Results

Expression of sGn-H Monomer and sGn-H-FT Nanoparticle

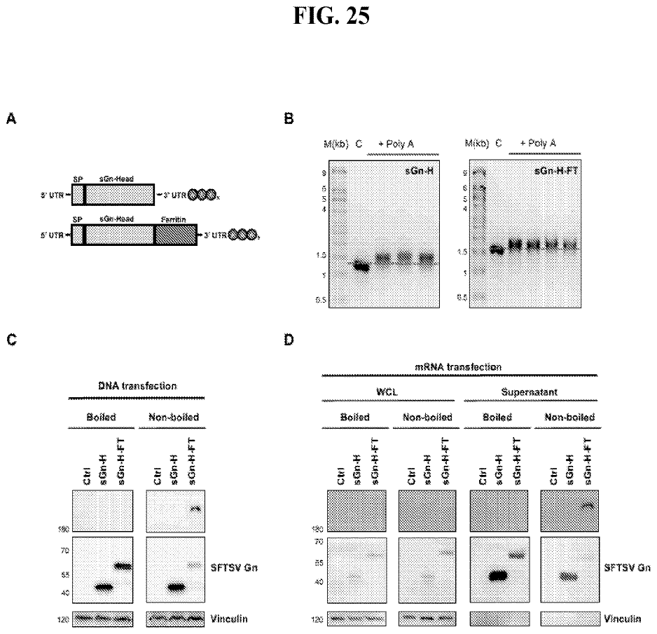

The SFTSV Gn Head region (sGn-H) not only plays an important role in attaching to the host cell receptor, CCR2 (22), but also is the critical target epitope of NAbs in convalescent human sera (23, 24). To ensure efficient expression, sGn-H and sGn-H-FT were codon-optimized to match human codon usage and cloned into a vector containing the 5′ untranslated region (UTR), signal peptide (SP), and 3′ UTR. Linearized and amplified DNA templates were utilized for co-transcriptional N1-Methyl-PseudoUTP incorporation and 5′ capping. The resulting in vitro transcribed mRNA was enzymatically polyadenylated (FIG. 1A). The mRNA products before and after polyadenylation underwent purification and size analysis through MOPS/formaldehyde RNA gel electrophoresis. The results showed the efficient polyadenylation of sGn-H and sGn-H-FT mRNAs (FIG. 25B).

HEK293T cells were transfected with mammalian vector to characterize sGn-H or sGn-H-FT expression. Supernatants from transfected cells were collected and analyzed for their expressions by immunoblot with anti-Gn antibody. The samples were subsequently loaded onto the gel with or without boiling step to assess the oligomerization of sGn-H-FT 24-mer nanoparticles. This showed efficient expressions of sGn-H and sGn-H-FT with the expected molecular weights of 45 kDa and 65 kDa, respectively, under boiled and non-boiled conditions (FIG. 25C). Notably, a high molecular weight sGn-H-FT was present close to the stacking gel of SDS-PAGE upon non-boiling conditions, suggesting the retention of 24-mer nanoparticle homopolymer structure as previously shown (26). Cells transfected with the 5′-capped and polyadenylated mRNAs also showed efficient expressions of sGn-H and sGn-H-FT in their whole cell lysates (WLC) and supernatants with or without boiling condition (FIG. 25D). Comparison of band intensities between WCL and supernatants indicated that most of sGn-H and sGn-H-FT were secreted from transfected cells, consistently with our previous study (26) (FIG. 25D). Equivalent levels of vinculin, serving as a loading control, were present except the supernatant fractions (FIG. 25C,D). Collectively, these data indicate that sGn-H and sGn-H-FT mRNA formats display efficient expressions in transfected cells.

sGn-H and sGn-H-FT mRNA LNP Vaccination Induces Strong Humoral Immunity in Immunized Mice

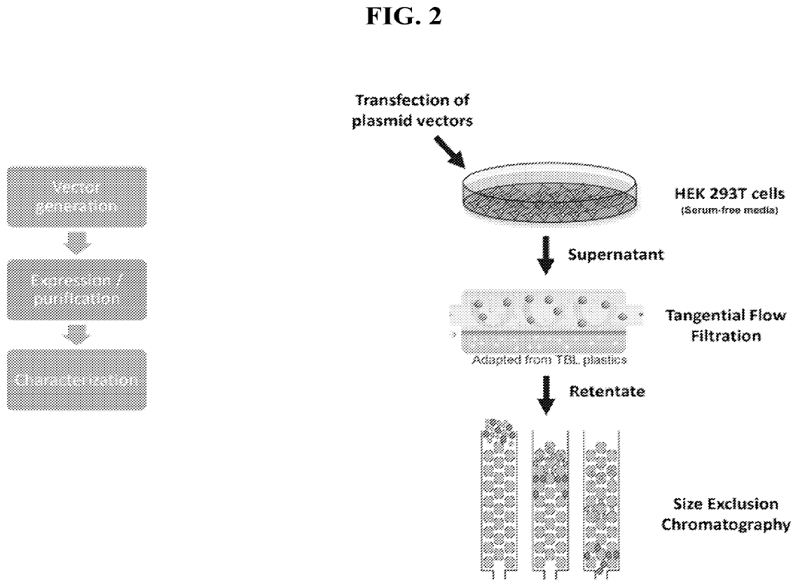

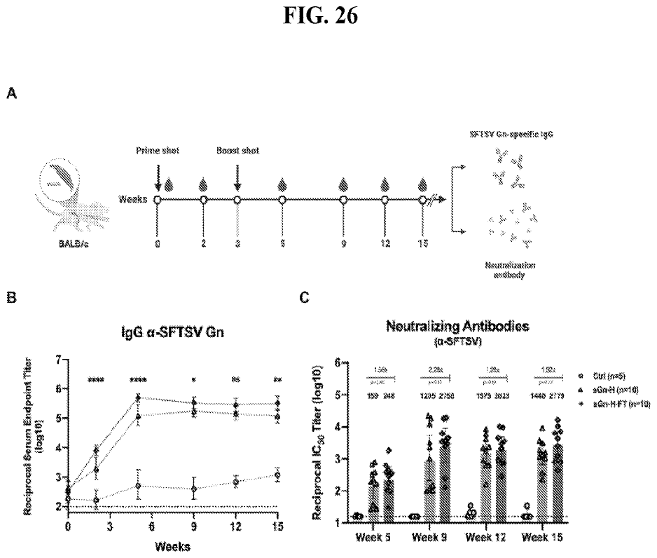

sGn-H or sGn-H-FT mRNAs were encapsulated into lipid nanoparticles (LNP) with approximately 90-95% efficiency for mRNA delivery. Six to eight-week-old BALB/c mice were immunized with 1 μg of sGn-H mRNA LNPs (n=10) or sGn-H-FT (n=10) mRNA LNPs, or PBS (n=5) as control at weeks 0 and 3 (FIG. 2A). Purified sGn-H was coated to ELISA plates to measure total antibody response and recombinant vesicular stomatitis virus (rVSV-SFTSV G-Luc) carrying SFTSV Gn-Gc glycoprotein and luciferase reporter genes was used for pseudovirus neutralization assay as previously described (26). Blood samples were collected at weeks 0, 2, 5, 9, 12, and 15 to evaluate overall sGn-H-recognizing total antibody and NAb levels over 15 weeks. Mice immunized with sGn-H or sGn-H-FT mRNA LNPs showed significant increases of anti-sGn-H total IgG after the first immunization with further elevation after the second immunization (FIG. 26B). Due to the enhanced immunogenicity achieved by sGn-H-FT nanoparticle, overall anti-sGn-H antibody titers in mice immunized with sGn-H-FT mRNA LNPs were detectably higher than those in mice immunized with sGn-H mRNA LNPs at weeks 2, 5, 9, and 15. Remarkably, anti-sGn-H antibody titers remained high, even at week 15 (12 weeks after the second immunization), indicating potential prolonged protection following immunization with sGn-H or sGn-H-FT mRNA LNPs. In contrast, PBS control group did not exhibit any detectable induction of anti-sGn-H IgG in ELISA after immunizations (FIG. 26B). Mice immunized with sGn-H mRNA or sGn-H-FT mRNA LNPs showed robust inductions of NAbs against SFTSV infection. Interestingly, NAb titers continuously increased until week 9 (six weeks after the second immunization) (FIG. 26C). Similarly, NAb titers from mice immunized with sGn-H-FT mRNA LNPs were 1.56-fold (week 5), 2.28-fold (week 9), 1.28-fold (week 12), and 1.92-fold (week 15) higher in geometric mean titers than those in mice immunized with sGn-H mRNA LNPs, although these differences were statistically insignificant (FIG. 26C). In contrast, PBS control group did not exhibit any detectable induction of NAbs after immunizations (FIG. 26B). Collectively, these results show the strong activity of sGn-H and sGn-H-FT mRNA LNPs to induce effective humoral immunity in vaccinated mice.

Immunization with sGn-H mRNA or sGn-H-FT mRNA LNPs Provides Protective Immunity Against Lethal SFTSV Infection

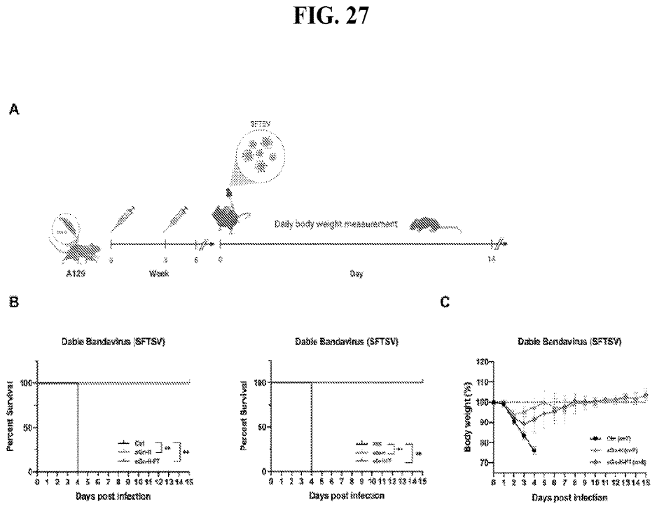

A129 IFNαβR^{-/-} mouse model has been developed for SFTSV infection and pathogenesis (refs). A129 mice were immunized at weeks 0 and 3 with PBS control, sGn-H mRNA LNPs, or sGn-H-FT mRNA LNPs at a dose of 3 μg. At two weeks after the second immunization, mice were infected with a lethal dose of SFTSV at 2E4 plaque-forming units (pfu) and monitored for clinical symptoms of fatality and body weight loss for 15 days post-infection (dpi) (FIG. 27A). Mice immunized with sGn-H or sGn-H-FT mRNA LNPs were fully protected from lethal challenge, whereas mice in PBS control group all succumbed to fatal infection by 4 dpi (FIG. 27B). Consistently, mice immunized with sGn-H mRNA or sGn-H-FT mRNA LNPs did not show body weight loss throughout post-infection period, but mice in PBS control group exhibited rapid body weight loss (FIG. 27C). These results show the strong protection efficacy of sGn-H and sGn-H-FT mRNA LNPs in A129 mouse model.

### REFERENCES FOR EXAMPLE 3

1. Casel et al., Severe fever with thrombocytopenia syndrome virus: emerging novel phlebovirus and their control strategy. Experimental & Molecular Medicine. 2021; 53 (5): 713-22.
2. Kwang-Min et al., Cross-genotype protection of live-attenuated vaccine candidate for severe fever with thrombocytopenia syndrome virus in a ferret model. PNAS. 2019; 116 (52): 26900-8.
3. Yu et al. Fever with thrombocytopenia associated with a novel bunyavirus in China. N Engl J Med. 2011; 364 (16): 1523-32.
4. Zhang et al. Hemorrhagic fever caused by a novel Bunyavirus in China: pathogenesis and correlates of fatal outcome. Clin Infect Dis. 2012; 54 (4): 527-33.
5. Yoshikawa et al. Phylogenetic and Geographic Relationships of Severe Fever With Thrombocytopenia Syndrome Virus in China, South Korea, and Japan. J Infect Dis. 2015; 212 (6): 889-98.
6. Yoo et al., Application of therapeutic plasma exchange in patients having severe fever with thrombocytopenia syndrome. Korean J Intern Med. 2019; 34 (4): 902-9.
7. Yun et al. Genetic and pathogenic diversity of severe fever with thrombocytopenia syndrome virus (SFTSV) in South Korea. JCI Insight. 2020; 5 (2).
8. Lam et al. Evolutionary and molecular analysis of the emergent severe fever with thrombocytopenia syndrome virus. Epidemics. 2013; 5 (1): 1-10.
9. Li et al., Severe fever with thrombocytopenia syndrome virus: a highly lethal bunyavirus. Crit Rev Microbiol. 2021; 47 (1): 112-25.
10. Jiang et al. A cluster of person-to-person transmission cases caused by SFTS virus in Penglai, China. Clin Microbiol Infect. 2015; 21 (3): 274-9.
11. Yun et al. Molecular genomic characterization of tick- and human-derived severe fever with thrombocytopenia syndrome virus isolates from South Korea. PLOS Negl Trop Dis. 2017; 11 (9): e0005893.
12. Zhang et al. Rapid Spread of Severe Fever with Thrombocytopenia Syndrome Virus by Parthenogenetic Asian Longhorned Ticks. Emerg Infect Dis. 2022; 28 (2): 363-72.
13. Animal and Plant Health Inspection Service USDoA. National Haemaphysalis longicornis (Asian longhorned tick) Situation Report. aphis.usda.gov2023.
14. (CDC) CfDCaP. What you need to know about Asian longhorned ticks-a new tick in the United States2023. Available from: https://www.cdc.gov/ticks/longhorned-tick/index.html.
15. Diseases NIoAal. NIAID Emerging Infectious Diseases/Pathogens-Category C. In: Diseases BaEI, editor. 2018.
16. Annual review of diseases prioritized under the Research and Development Blueprint, Informal consultation. World Health Organization; 2018.
17. Li et al. Molecular evolution and genetic diversity analysis of SFTS virus based on next-generation sequencing. Biosafety and Health. 2021; 3 (2): 105-15.
18. Sun et al. Architecture of severe fever with thrombocytopenia syndrome virus. Protein & Cell. 2023.
19. Xia et al. Glucosylceramide is essential for Heartland and Dabie bandavirus glycoprotein-induced membrane fusion. PLOS Pathogens. 2023; 19 (3): e1011232.
20. Jia et al. Identification of a candidate standard strain of severe fever with thrombocytopenia syndrome virus for vaccine quality control in China using a cross-neutralization assay. Biologicals. 2017; 46:92-8.
21. Plegge et al., Evidence that Processing of the Severe Fever with Thrombocytopenia Syndrome Virus Gn/Gc Polyprotein Is Critical for Viral Infectivity and Requires an Internal Gc Signal Peptide. PLOS One. 2016; 11 (11): e0166013.
22. Zhang et al. CCR2 is a host entry receptor for severe fever with thrombocytopenia syndrome virus. Science Advances. 2023; 9 (31): eadg6856.
23. Guo et al. Human antibody neutralizes severe Fever with thrombocytopenia syndrome virus, an emerging hemorrhagic Fever virus. Clin Vaccine Immunol. 2013; 20 (9): 1426-32.
24. Kim et al. An anti-Gn glycoprotein antibody from a convalescent patient potently inhibits the infection of severe fever with thrombocytopenia syndrome virus. PLOS Pathog. 2019; 15 (2): e1007375.
25. Kwak et al. Development of a SFTSV DNA vaccine that confers complete protection against lethal infection in ferrets. Nature Communications. 2019; 10 (1): 3836.
26. Kim et al. Self-assembling Gn head ferritin nanoparticle vaccine provides full protection from lethal challenge of Dabie bandavirus in aged ferrets. mBio. 2023: e0186823.
27. Pollard A J, Bijker E M. A guide to vaccinology: from basic principles to new developments. Nature Reviews Immunology. 2021; 21 (2): 83-100.
28. Gause et al . . . , Immunological Principles Guiding the Rational Design of Particles for Vaccine Delivery. ACS Nano. 2017; 11 (1): 54-68.
29. Kelly et al. Self-assembling influenza nanoparticle vaccines drive extended germinal center activity and memory B cell maturation. JCI Insight. 2020; 5 (10).
30. Kanekiyo et al. Rational Design of an Epstein-Barr Virus Vaccine Targeting the Receptor-Binding Site. Cell. 2015; 162 (5): 1090-100.
31. Kim et al. Development of Spike Receptor-Binding Domain Nanoparticles as a Vaccine Candidate against SARS-COV-2 Infection in Ferrets. mBio. 2021; 12 (2).
32. Kim et al. Chaperna-Mediated Assembly of Ferritin-Based Middle East Respiratory Syndrome-Coronavirus Nanoparticles. Front Immunol. 2018; 9:1093.
33. Park et al. Ferret animal model of severe fever with thrombocytopenia syndrome phlebovirus for human lethal infection and pathogenesis. Nat Microbiol. 2019; 4 (3): 438-46.
34. Li et al. Safety and immunogenicity of the SARS-COV-2 BNT162b1 mRNA vaccine in younger and older Chinese adults: a randomized, placebo-controlled, double-blind phase 1 study. Nature Medicine. 2021; 27 (6): 1062-70.
35. Brailovskaia et al., To vaccinate or not to vaccinate!? Predictors of willingness to receive Covid-19 vaccination in Europe, the U.S., and China. PLOS ONE. 2021; 16 (12): e0260230.
36. Jackson et al., The promise of mRNA vaccines: a biotech and industrial perspective. npj Vaccines. 2020; 5 (1): 11.
37. Pardi et al., mRNA vaccines-a new era in vaccinology. Nature Reviews Drug Discovery. 2018; 17 (4): 261-79.
38. Baden et al. Efficacy and Safety of the mRNA-1273 SARS-COV-2 Vaccine. New England Journal of Medicine. 2020; 384 (5): 403-16.
39. Polack et al. Safety and Efficacy of the BNT162b2 mRNA Covid-19 Vaccine. New England Journal of Medicine. 2020; 383 (27): 2603-15.
40. Watson et al., Global impact of the first year of COVID-19 vaccination: a mathematical modelling study. Lancet Infect Dis. 2022; 22 (9): 1293-302.
41. Wu et al. Long-term effectiveness of COVID-19 vaccines against infections, hospitalisations, and mortality in adults: findings from a rapid living systematic evidence synthesis and meta-analysis up to December 2022. Lancet Respir Med. 2023; 11 (5): 439-52.
42. Barbier et al., The clinical progress of mRNA vaccines and immunotherapies. Nature Biotechnology. 2022; 40 (6): 840-54.
43. Richner et al. Modified mRNA Vaccines Protect against Zika Virus Infection. Cell. 2017; 168 (6): 1114-25 e10.
44. Kimura et al. Characterization of pseudotyped vesicular stomatitis virus bearing the heartland virus envelope glycoprotein. Virology. 2021; 556:124-32.
45. Yu et al. Seroprevalence and genetic characterization of severe fever with thrombocytopenia syndrome virus in domestic goats in South Korea. Ticks Tick Borne Dis. 2018; 9 (5): 1202-6.
46. LaMori et al., Hepatitis vaccination adherence and completion rates and factors associated with low compliance: A claims-based analysis of U.S. adults. PLOS One. 2022; 17 (2): e0264062.
47. Sandoval et al., Effectiveness of mRNA, protein subunit vaccine and viral vectors vaccines against SARS-COV-2 in people over 18 years old: a systematic review. Expert Review of Vaccines. 2023; 22 (1): 35-53.
48. Matheson et al, J. Increasing HPV Vaccination Series Completion Rates via Text Message Reminders. Journal of Pediatric Health Care. 2014; 28 (4): e35-e9.
49. Zhang et al. Rapid development of an updated mRNA vaccine against the SARS-COV-2 Omicron variant. Cell Research. 2022; 32 (4): 401-3.
50. Abu, et al. Effect of mRNA Vaccine Boosters against SARS-COV-2 Omicron Infection in Qatar. New England Journal of Medicine. 2022; 386 (19): 1804-16.

All publications and patents mentioned in the specification and/or listed below are herein incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the relevant fields are intended to be within the scope described herein.

We claim:

1. A composition comprising: a plurality of fusion proteins, and/or a polynucleotide encoding said fusion protein, wherein each of said plurality of fusion proteins comprises:
a) at least a portion of a Ferritin protein, and
b) an immunogenic protein comprising at least a portion of: i) severe fever with thrombocytopenia syndrome virus (SFTS) virus Gn envelope glycoprotein and/or ii) SFTS virus Gc envelope glycoprotein,
wherein said plurality of fusion proteins are self-assembled into a plurality of nanoparticles, and
wherein said plurality of nanoparticles each display said immunogenic protein on their surfaces.

2. The composition of claim 1, wherein said composition further comprises an adjuvant.

3. The composition of claim 1, wherein said at least a portion of said Ferritin protein comprises at least 25 consecutive amino acids from a Ferritin protein.

4. The composition of claim 1, wherein said at least a portion of said Ferritin protein comprise at least 25 consecutive amino acids, or at least 100 consecutive amino acids, or at least about 150 consecutive amino acids, or the entire amino acid sequence, from an amino acid sequence selected from: SEQ ID NOs: 4, 5, 6, 7, and 8.

5. The composition of claim 1, wherein said at least a portion of said Ferritin protein comprises an amino acid sequence at least about 90% identical, or at least 95% identical, or at least 99% identical, or 100% identical, to an amino acid sequence selected from SEQ ID NOs: 4, 5, 6, 7, and 8.

6. The composition of claim 1, wherein said at least a portion of said Ferritin protein comprises an amino acid sequence selected from SEQ ID NOs: 4, 5, 6, 7, 8 or SEQ ID NOs: 4, 5, 6, 7, and 8 with one or two conservative amino acid changes.

7. The composition of claim 1, wherein said at least a portion of said Ferritin protein is a hybrid protein comprising at least a portion of a bullfrog ferritin protein joined to at least a portion of a ferritin protein selected from the group consisting of a Helicobacter pylori ferritin protein and an Escherichia coli ferritin protein.

8. The composition of claim 1, wherein said at least a portion of said SFTS virus Gn envelope glycoprotein comprises at least 25, or at least 50, consecutive amino acids, or the entire amino acid sequence; from an amino acid sequence selected from: SEQ ID NOs: 2, 9, 11, 25, and 27, or SEQ ID NOs: 2, 9, 11, 25, and 27 with one or two conservative amino acid changes or end deletions.

9. The composition of claim 1, wherein said at least a portion of said SFTS virus Gc envelope glycoprotein comprises at least 25 consecutive amino acids, or at least 100 consecutive amino acids, or at least about 200 consecutive amino acids, or the entire amino acid sequence; from an amino acid sequence selected from: SEQ ID NOs: 3, 13, and 15, or SEQ ID NOs: 3, 13 and 15 with one or two conservative amino acid changes.

10. The composition of claim 1, wherein each of said fusion proteins further comprises a linker sequence.

11. The composition of claim 1, wherein said polynucleotide comprises at least 24 or 35 consecutive nucleotides from any of SEQ ID NOs: 1, 10, 12, 14, 16, 18, 20, 22, 24, 26, 31, and 33-40, and wherein T is optionally replaced with U, and/or any of the nucleotides, including U, are replaced with a modified base.

12. The composition of claim 1, wherein said polynucleotide comprises RNA.

13. The composition of claim 1, wherein said polynucleotide is partially or fully human codon optimized, or HSV-1 glycoprotein B codon optimized, and is optionally RNA.

14. The composition of claim 1, wherein the polynucleotide further comprises or encodes: a 5′ untranslated region (UTR), a 5′ cap, a 3′ UTR, an IRES, a 3′ tailing sequence, or any combination thereof.

15. The composition of claim 14, wherein the 3′ tailing sequence comprises a polyA tail, a polyG quartet, a stem loop sequence, a triple helix forming sequence, a tRNA-like sequence, or any combination thereof.

16. The composition of claim 1, wherein said polynucleotide comprises at least one chemically modified nucleotide.

17. The composition of claim 16, wherein the at least one chemically modified nucleotide comprises a modified uracil.

18. The composition of claim 17, wherein at least 60% of the uracils in the polynucleotide are chemically modified.

19. The composition of claim 16, wherein the at least one chemically modified nucleotide comprises 5-methylcytosine or N1-methylpseudouridine (m1Ψ).

20. The composition of claim 1, wherein said polynucleotide comprises a nucleotides sequence: i) having at least 75%, or 85%, or 95% identity to SEQ ID NOs: 1, 10, 12, 14, 16, 18, 20, 22, 24, 26, 31, or 33-40, or a complement or reverse complement thereof, wherein T may be replaced by U or a modified U, and/or any of the nucleotides, including U, are replaced with a modified base.

21. A method of immunizing a subject comprising at least one of the following:
a) administering at least a portion of said plurality of nanoparticles of any of claims 1-10 to a subject such that an immune response to said immunogenic protein is produced in said subject, and/or
b) administering said polynucleotide of any of claims 1-20, optionally present in an expression vector or delivery vehicle, such that said nanoparticles are expressed in said subject and such that an immune response to said immunogenic protein is product in said subject.

22. The method of claim 21, wherein said subject is a human.

23. The method of claim 21, wherein the delivery vehicle comprises a lipid nanoparticle encapsulating the composition.

24. The method of claim 23, wherein the lipid nanoparticle comprises a cationic lipid, a neutral and/or non-cationic lipid, a sterol, or any combination thereof.


SEQUENCE LISTING (40 sequences)

SEQ ID NO: 1 - DNA, 960 bp, Synthetic construct
  source 1..960: mol_type = other DNA
accggtcctatcatctgtgccggacccatccacagcaacaagagcgccgacatcccccatctgctgggctacagcgagaagatttgccagatcgaccggctgatccacgtgtcctcttggctgcggaaccacagccagttccagggctacgtgggacagagaggcggcagatcccaggtgtcctactacccagctgaaaacagctacagccggtggagcggcctgctgagcccttgtgatgctgactggctgggcatgctggtcgtgaagaaggccaagggcagcgacatgatcgtgcctggccctagctacaagggcaaggtgttcttcgagcggcccaccttcgacggctatgtgggctggggatgtggcagcggcaagagcagaacagagtccggcgagctgtgcagcagcgattctggcacaagctctggcctgctgcccagcgacagagtgctgtggattggcgacgtggcctgccagcccatgacccctatccccgaggaaacctttctggaactgaagtccttcagccagagcgagttccccgacatctgcaagatcgacggcatcgtgttcaaccagtgcgagggcgagagcctgccccagccttttgatgtggcctggatggacgtgggccactcccacaagatcatcatgcgcgagcacaagaccaaatgggtgcaggaaagcagcagcaaggacttcgtgtgctacaaagagggcaccggcccctgcagcgagagcgaggaaaagacctgcaagaccagcggctcctgcagaggcgacatgcagttctgcaaggtggccggatgcgagcacggcgaagaggccagcgaggccaagtgcagatgtagcctggtgcacaagcccggcgaggtggtggtgtcttacggcggaatgagagtgcggcccaagtgctacggcttcagccggatgatggccaccctggaagtgaac

SEQ ID NO: 2 - protein, 516 aa, Dabie bandavirus
DSGPIICAGPIHSNKSAGIPHLLGYSEKICQIDRLIHVSSWLRNHSQFQGYVGQRGGRSQVSYYPAENSYSRWSGLLSPCDADWLGMLVVKKAKESDMIVPGPSYKGKVFFERPTFDGYVGWGCGSGKSRTESGELCSSDSGTSSGLLPSDRVLWIGDVACQPMTPIPEETFLELKSFSQSEFPDICKIDGIVFNQCEGESLPQPFDVAWMDVGHSHKIIMREHKTKWVQESSSKDFVCYKEGTGPCSESEEKACKTSGSCRGDMQFCKVAGCEHGEEASEAKCRCSLVHKPGEVVVSYGGTRVRPKCYGFSRMMATLEVNPPEQRIGQCTGCHLECINGGVRLITLTSELRSATVCASHFCSSASSGKKSTEIHFHSGSLVGKTAIHVKGALVDGTEFTFEGSCMFPDGCDAVDCTFCREFLKNPQCYPAKKWLFIIIVILLGYAGLMLLTNVLKAIGVWGSWVIAPVKLMFAIIKKLMRTVSCLVGKLMDRGRQVIHEEIGENGEGNQDDVRIE

SEQ ID NO: 3 - protein, 511 aa, Dabie bandavirus
CDEMVHADSKLVSCRQGSGNMKECITTGRALLPAVNPGQEACLHFTAPGSPDSKCLKIKVKRINLKCKKSSSYFVPDARSRCTSVRRCRWAGDCQSGCPPHFTSNSFSDDWAGKMDRAGLGFSGCSDGCGGAACGCFNAAPSCIFWRKWVENPHGIIWKVSPCAAWVPSAVIELTMPSGEVRTFHPMSGIPTQVFKGVSVTYLGSDMEVSGLTDLCEIEELKSKKLALAPCNQAGMGVVGKVGEIQCSSEESARTIKKDGCIWNADLVGIELRVDDAVCYSKITSVEAVANYSAIPTTIGGLRFERSHDSQGKISGSPLDITAIRGSFSVNYRGLRLSLSEITATCTGEVTNVSGCYSCMTGAKVSIKLHSSKNSTAHVRCKGDETAFSVLEGVHSYIVRLSFDHAVVDEQCQLNCGGHESQVTLKGNLIFLDVPKFVDGSYMQTYHSTVPTGANIPSPTDWLNALFGNGLSRWILGVIGVLLGGLALFFLIMFLLKLGTKQVFRSRTKLA

SEQ ID NO: 4 - protein, 167 aa, Helicobacter pylori
MLSKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKVELIGNENHGLYLADQYVKGIAKSRKS

SEQ ID NO: 5 - protein, 165 aa, Escherichia coli
MLKPEMIEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDAQN

SEQ ID NO: 6 - protein, 173 aa, Lithobates catesbeianus
MESQVRQNFHQDCEAGLNRTVNLKFHSSYVYLSMASYFNRDDVALSNFAKFFRERSEEEKEHAEKLIEYQNQRGGRVFLQSVEKPERDDWANGLEALQTALKLQKSVNQALLDLHAVAADKSDPHMTDFLESPYLSESVETIKKLGDHITSLKKLWSSHPGMAEYLFNKHTLG

SEQ ID NO: 7 - protein, 177 aa, Synthetic construct
SGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 8 - protein, 172 aa, Synthetic construct
SGESQVRQNFKPEMEEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDGS

SEQ ID NO: 9 - protein, 320 aa, Synthetic construct
TGPIICAGPIHSNKSADIPHLLGYSEKICQIDRLIHVSSWLRNHSQFQGYVGQRGGRSQVSYYPAENSYSRWSGLLSPCDADWLGMLVVKKAKGSDMIVPGPSYKGKVFFERPTFDGYVGWGCGSGKSRTESGELCSSDSGTSSGLLPSDRVLWIGDVACQPMTPIPEETFLELKSFSQSEFPDICKIDGIVFNQCEGESLPQPFDVAWMDVGHSHKIIMREHKTKWVQESSSKDFVCYKEGTGPCSESEEKTCKTSGSCRGDMQFCKVAGCEHGEEASEAKCRCSLVHKPGEVVVSYGGMRVRPKCYGFSRMMATLEVN

SEQ ID NO: 10 - DNA, 1299 bp, Synthetic construct
  source 1..1299: mol_type = other DNA
accggtcctatcatctgtgccggacccatccacagcaacaagagcgccgacatcccccatctgctgggctacagcgagaagatttgccagatcgaccggctgatccacgtgtcctcttggctgcggaaccacagccagttccagggctacgtgggacagagaggcggcagatcccaggtgtcctactacccagctgaaaacagctacagccggtggagcggcctgctgagcccttgtgatgctgactggctgggcatgctggtcgtgaagaaggccaagggcagcgacatgatcgtgcctggccctagctacaagggcaaggtgttcttcgagcggcccaccttcgacggctatgtgggctggggatgtggcagcggcaagagcagaacagagtccggcgagctgtgcagcagcgattctggcacaagctctggcctgctgcccagcgacagagtgctgtggattggcgacgtggcctgccagcccatgacccctatccccgaggaaacctttctggaactgaagtccttcagccagagcgagttccccgacatctgcaagatcgacggcatcgtgttcaaccagtgcgagggcgagagcctgccccagccttttgatgtggcctggatggacgtgggccactcccacaagatcatcatgcgcgagcacaagaccaaatgggtgcaggaaagcagcagcaaggacttcgtgtgctacaaagagggcaccggcccctgcagcgagagcgaggaaaagacctgcaagaccagcggctcctgcagaggcgacatgcagttctgcaaggtggccggatgcgagcacggcgaagaggccagcgaggccaagtgcagatgtagcctggtgcacaagcccggcgaggtggtggtgtcttacggcggaatgagagtgcggcccaagtgctacggcttcagccggatgatggccaccctggaagtgaaccagcccgagcagagaatcggccagtgtaccggctgccacctggaatgcatcaatggcggcgtgcggctgatcaccctgaccagcgaactgaaaagcgccaccgtgtgcgccagccacttctgtagcagcgccacctccggcaagaagtccaccgagatccagttccacagcggcagcctcgtgggcaagacagccatccatgtgaagggggccctggtggacggcaccgaattcacctttgagggctcttgcatgttccccgatggctgcgacgccgtggactgtaccttctgcagagagttcctgaagaacccccagtgctaccccgccaagaaatgg

SEQ ID NO: 11 - protein, 433 aa, Synthetic construct
TGPIICAGPIHSNKSADIPHLLGYSEKICQIDRLIHVSSWLRNHSQFQGYVGQRGGRSQVSYYPAENSYSRWSGLLSPCDADWLGMLVVKKAKGSDMIVPGPSYKGKVFFERPTFDGYVGWGCGSGKSRTESGELCSSDSGTSSGLLPSDRVLWIGDVACQPMTPIPEETFLELKSFSQSEFPDICKIDGIVFNQCEGESLPQPFDVAWMDVGHSHKIIMREHKTKWVQESSSKDFVCYKEGTGPCSESEEKTCKTSGSCRGDMQFCKVAGCEHGEEASEAKCRCSLVHKPGEVVVSYGGMRVRPKCYGFSRMMATLEVNQPEQRIGQCTGCHLECINGGVRLITLTSELKSATVCASHFCSSATSGKKSTEIQFHSGSLVGKTAIHVKGALVDGTEFTFEGSCMFPDGCDAVDCTFCREFLKNPQCYPAKKW

SEQ ID NO: 12 - DNA, 1533 bp, Synthetic construct
  source 1..1533: mol_type = other DNA
tgtgacgaaatggtgcacgccgacagcaaactggtgtcctgcagacagggctccggcaacatgaaggaatgcgtgaccaccggcagagccctgctgcctgctgtgaaccctggacaggaagcctgcctgcactttaccgcccctggcagccctgacagcaagtgcctgaagatcaaagtgaagcggatcaacctgaagtgcaagaaaagcagctcctacttcgtgcccgacgcccggtccagatgtaccagcgtgcggagatgtagatgggcaggcgattgccagagcggctgccctcctcacttcaccagcaacagcttcagcgacgactgggccggcaagatggacagagccggactgggctttagcggctgctctgatggatgtggcggagccgcctgcggctgctttaatgccgcccctagctgcatcttctggcggaaatgggtggaaaacccccacggcatcatctggaaggtgtccccatgtgccgcctgggtgccatctgccgtgatcgagctgacaatgcccagcggcgaagtgcggaccttccaccctatgagcggcatccccacccaggtgttcaagggcgtgtccgtgacatacctgggctccgacatggaagtgtccggcctgaccgacctgtgcgagattgaggaactgaaatccaagaagctggccctggccccctgtaaccaggccggaatgggagtcgtgggaaaagtgggcgagattcagtgcagctccgaggaaagcgcccggaccatcaagaaggacggctgcatctggaacgccgacctcgtgggaattgagctgagagtggatgacgccgtgtgttacagcaagatcaccagcgtggaagccgtggccaactacagcgccatccctaccacaatcggcggcctgagattcgagcggagccacgatagccagggcaagatcagcggaagccccctggacatcaccgccatcaggggcagctttagcgtgaactaccggggcctgagactgagcctgagcgagatcacagccacctgtaccggggaagtgaccaatgtgtccggctgctactcctgcatgaccggcgccaaggtgtccatcaagctgcactccagcaagaacagcaccgcccacgtgcggtgcaagggcgacgagacagccttctctgtgctggaaggcgtgcacagctacaccgtgtccctgagcttcgaccacgccgtggtggatgagcagtgccaactgaactgtggcggccacgagtcccaagtgaccctgaagggaaacctgatcttcctggacgtgcccaagttcgtggacggctcctacatgcagacctaccacagcaccgtgcccacaggcgccaacatccctagccctaccgactggctgaacgccctgttcggcaacggcctgagcagatggatcctgggcgtgatcggagtgctgctgggaggactggccctgttcttcctgatcatgagcctgttcaagctgggcaccaaacaggtgttccggtcccggacaaagctggcc

SEQ ID NO: 13 - protein, 511 aa, Synthetic construct
CDEMVHADSKLVSCRQGSGNMKECVTTGRALLPAVNPGQEACLHFTAPGSPDSKCLKIKVKRINLKCKKSSSYFVPDARSRCTSVRRCRWAGDCQSGCPPHFTSNSFSDDWAGKMDRAGLGFSGCSDGCGGAACGCFNAAPSCIFWRKWVENPHGIIWKVSPCAAWVPSAVIELTMPSGEVRTFHPMSGIPTQVFKGVSVTYLGSDMEVSGLTDLCEIEELKSKKLALAPCNQAGMGVVGKVGEIQCSSEESARTIKKDGCIWNADLVGIELRVDDAVCYSKITSVEAVANYSAIPTTIGGLRFERSHDSQGKISGSPLDITAIRGSFSVNYRGLRLSLSEITATCTGEVTNVSGCYSCMTGAKVSIKLHSSKNSTAHVRCKGDETAFSVLEGVHSYTVSLSFDHAVVDEQCQLNCGGHESQVTLKGNLIFLDVPKFVDGSYMQTYHSTVPTGANIPSPTDWLNALFGNGLSRWILGVIGVLLGGLALFFLIMSLFKLGTKQVFRSRTKLA

SEQ ID NO: 14 - DNA, 1302 bp, Synthetic construct
  source 1..1302: mol_type = other DNA
tgtgacgaaatggtgcacgccgacagcaaactggtgtcctgcagacagggctccggcaacatgaaggaatgcgtgaccaccggcagagccctgctgcctgctgtgaaccctggacaggaagcctgcctgcactttaccgcccctggcagccctgacagcaagtgcctgaagatcaaagtgaagcggatcaacctgaagtgcaagaaaagcagctcctacttcgtgcccgacgcccggtccagatgtaccagcgtgcggagatgtagatgggcaggcgattgccagagcggctgccctcctcacttcaccagcaacagcttcagcgacgactgggccggcaagatggacagagccggactgggctttagcggctgctctgatggatgtggcggagccgcctgcggctgctttaatgccgcccctagctgcatcttctggcggaaatgggtggaaaacccccacggcatcatctggaaggtgtccccatgtgccgcctgggtgccatctgccgtgatcgagctgacaatgcccagcggcgaagtgcggaccttccaccctatgagcggcatccccacccaggtgttcaagggcgtgtccgtgacatacctgggctccgacatggaagtgtccggcctgaccgacctgtgcgagattgaggaactgaaatccaagaagctggccctggccccctgtaaccaggccggaatgggagtcgtgggaaaagtgggcgagattcagtgcagctccgaggaaagcgcccggaccatcaagaaggacggctgcatctggaacgccgacctcgtgggaattgagctgagagtggatgacgccgtgtgttacagcaagatcaccagcgtggaagccgtggccaactacagcgccatccctaccacaatcggcggcctgagattcgagcggagccacgatagccagggcaagatcagcggaagccccctggacatcaccgccatcaggggcagctttagcgtgaactaccggggcctgagactgagcctgagcgagatcacagccacctgtaccggggaagtgaccaatgtgtccggctgctactcctgcatgaccggcgccaaggtgtccatcaagctgcactccagcaagaacagcaccgcccacgtgcggtgcaagggcgacgagacagccttctctgtgctggaaggcgtgcacagctacaccgtgtccctgagcttcgaccacgccgtggtggatgagcagtgccaactgaactgtggcggccacgagtcccaagtgaccctgaagggaaacctgatcttcctggacgtg

SEQ ID NO: 15 - protein, 435 aa, Synthetic construct
CDEMVHADSKLVSCRQGSGNMKECVTTGRALLPAVNPGQEACLHFTAPGSPDSKCLKIKVKRINLKCKKSSSYFVPDARSRCTSVRRCRWAGDCQSGCPPHFTSNSFSDDWAGKMDRAGLGFSGCSDGCGGAACGCFNAAPSCIFWRKWVENPHGIIWKVSPCAAWVPSAVIELTMPSGEVRTFHPMSGIPTQVFKGVSVTYLGSDMEVSGLTDLCEIEELKSKKLALAPCNQAGMGVVGKVGEIQCSSEESARTIKKDGCIWNADLVGIELRVDDAVCYSKITSVEAVANYSAIPTTIGGLRFERSHDSQGKISGSPLDITAIRGSFSVNYRGLRLSLSEITATCTGEVTNVSGCYSCMTGAKVSIKLHSSKNSTAHVRCKGDETAFSVLEGVHSYTVSLSFDHAVVDEQCQLNCGGHESQVTLKGNLIFLDVG

SEQ ID NO: 16 - DNA, 1521 bp, Synthetic construct
  source 1..1521: mol_type = other DNA
accggtcctatcatctgtgccggacccatccacagcaacaagagcgccgacatcccccatctgctgggctacagcgagaagatttgccagatcgaccggctgatccacgtgtcctcttggctgcggaaccacagccagttccagggctacgtgggacagagaggcggcagatcccaggtgtcctactacccagctgaaaacagctacagccggtggagcggcctgctgagcccttgtgatgctgactggctgggcatgctggtcgtgaagaaggccaagggcagcgacatgatcgtgcctggccctagctacaagggcaaggtgttcttcgagcggcccaccttcgacggctatgtgggctggggatgtggcagcggcaagagcagaacagagtccggcgagctgtgcagcagcgattctggcacaagctctggcctgctgcccagcgacagagtgctgtggattggcgacgtggcctgccagcccatgacccctatccccgaggaaacctttctggaactgaagtccttcagccagagcgagttccccgacatctgcaagatcgacggcatcgtgttcaaccagtgcgagggcgagagcctgccccagccttttgatgtggcctggatggacgtgggccactcccacaagatcatcatgcgcgagcacaagaccaaatgggtgcaggaaagcagcagcaaggacttcgtgtgctacaaagagggcaccggcccctgcagcgagagcgaggaaaagacctgcaagaccagcggctcctgcagaggcgacatgcagttctgcaaggtggccggatgcgagcacggcgaagaggccagcgaggccaagtgcagatgtagcctggtgcacaagcccggcgaggtggtggtgtcttacggcggaatgagagtgcggcccaagtgctacggcttcagccggatgatggccaccctggaagtgaacggatcctctggaggagctagcgtgctggcctccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagtccggaagc

SEQ ID NO: 17 - protein, 507 aa, Synthetic construct
TGPIICAGPIHSNKSADIPHLLGYSEKICQIDRLIHVSSWLRNHSQFQGYVGQRGGRSQVSYYPAENSYSRWSGLLSPCDADWLGMLVVKKAKGSDMIVPGPSYKGKVFFERPTFDGYVGWGCGSGKSRTESGELCSSDSGTSSGLLPSDRVLWIGDVACQPMTPIPEETFLELKSFSQSEFPDICKIDGIVFNQCEGESLPQPFDVAWMDVGHSHKIIMREHKTKWVQESSSKDFVCYKEGTGPCSESEEKTCKTSGSCRGDMQFCKVAGCEHGEEASEAKCRCSLVHKPGEVVVSYGGMRVRPKCYGFSRMMATLEVNGSSGGASVLASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 18 - DNA, 1860 bp, Synthetic construct
  source 1..1860: mol_type = other DNA
accggtcctatcatctgtgccggacccatccacagcaacaagagcgccgacatcccccatctgctgggctacagcgagaagatttgccagatcgaccggctgatccacgtgtcctcttggctgcggaaccacagccagttccagggctacgtgggacagagaggcggcagatcccaggtgtcctactacccagctgaaaacagctacagccggtggagcggcctgctgagcccttgtgatgctgactggctgggcatgctggtcgtgaagaaggccaagggcagcgacatgatcgtgcctggccctagctacaagggcaaggtgttcttcgagcggcccaccttcgacggctatgtgggctggggatgtggcagcggcaagagcagaacagagtccggcgagctgtgcagcagcgattctggcacaagctctggcctgctgcccagcgacagagtgctgtggattggcgacgtggcctgccagcccatgacccctatccccgaggaaacctttctggaactgaagtccttcagccagagcgagttccccgacatctgcaagatcgacggcatcgtgttcaaccagtgcgagggcgagagcctgccccagccttttgatgtggcctggatggacgtgggccactcccacaagatcatcatgcgcgagcacaagaccaaatgggtgcaggaaagcagcagcaaggacttcgtgtgctacaaagagggcaccggcccctgcagcgagagcgaggaaaagacctgcaagaccagcggctcctgcagaggcgacatgcagttctgcaaggtggccggatgcgagcacggcgaagaggccagcgaggccaagtgcagatgtagcctggtgcacaagcccggcgaggtggtggtgtcttacggcggaatgagagtgcggcccaagtgctacggcttcagccggatgatggccaccctggaagtgaaccagcccgagcagagaatcggccagtgtaccggctgccacctggaatgcatcaatggcggcgtgcggctgatcaccctgaccagcgaactgaaaagcgccaccgtgtgcgccagccacttctgtagcagcgccacctccggcaagaagtccaccgagatccagttccacagcggcagcctcgtgggcaagacagccatccatgtgaagggggccctggtggacggcaccgaattcacctttgagggctcttgcatgttccccgatggctgcgacgccgtggactgtaccttctgcagagagttcctgaagaacccccagtgctaccccgccaagaaatggggatcctctggaggagctagcgtgctggcctccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagtccggaagc

SEQ ID NO: 19 - protein, 620 aa, Synthetic construct
TGPIICAGPIHSNKSADIPHLLGYSEKICQIDRLIHVSSWLRNHSQFQGYVGQRGGRSQVSYYPAENSYSRWSGLLSPCDADWLGMLVVKKAKGSDMIVPGPSYKGKVFFERPTFDGYVGWGCGSGKSRTESGELCSSDSGTSSGLLPSDRVLWIGDVACQPMTPIPEETFLELKSFSQSEFPDICKIDGIVFNQCEGESLPQPFDVAWMDVGHSHKIIMREHKTKWVQESSSKDFVCYKEGTGPCSESEEKTCKTSGSCRGDMQFCKVAGCEHGEEASEAKCRCSLVHKPGEVVVSYGGMRVRPKCYGFSRMMATLEVNQPEQRIGQCTGCHLECINGGVRLITLTSELKSATVCASHFCSSATSGKKSTEIQFHSGSLVGKTAIHVKGALVDGTEFTFEGSCMFPDGCDAVDCTFCREFLKNPQCYPAKKWGSSGGASVLASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 20 - DNA, 1863 bp, Synthetic construct
  source 1..1863: mol_type = other DNA
tgtgacgaaatggtgcacgccgacagcaaactggtgtcctgcagacagggctccggcaacatgaaggaatgcgtgaccaccggcagagccctgctgcctgctgtgaaccctggacaggaagcctgcctgcactttaccgcccctggcagccctgacagcaagtgcctgaagatcaaagtgaagcggatcaacctgaagtgcaagaaaagcagctcctacttcgtgcccgacgcccggtccagatgtaccagcgtgcggagatgtagatgggcaggcgattgccagagcggctgccctcctcacttcaccagcaacagcttcagcgacgactgggccggcaagatggacagagccggactgggctttagcggctgctctgatggatgtggcggagccgcctgcggctgctttaatgccgcccctagctgcatcttctggcggaaatgggtggaaaacccccacggcatcatctggaaggtgtccccatgtgccgcctgggtgccatctgccgtgatcgagctgacaatgcccagcggcgaagtgcggaccttccaccctatgagcggcatccccacccaggtgttcaagggcgtgtccgtgacatacctgggctccgacatggaagtgtccggcctgaccgacctgtgcgagattgaggaactgaaatccaagaagctggccctggccccctgtaaccaggccggaatgggagtcgtgggaaaagtgggcgagattcagtgcagctccgaggaaagcgcccggaccatcaagaaggacggctgcatctggaacgccgacctcgtgggaattgagctgagagtggatgacgccgtgtgttacagcaagatcaccagcgtggaagccgtggccaactacagcgccatccctaccacaatcggcggcctgagattcgagcggagccacgatagccagggcaagatcagcggaagccccctggacatcaccgccatcaggggcagctttagcgtgaactaccggggcctgagactgagcctgagcgagatcacagccacctgtaccggggaagtgaccaatgtgtccggctgctactcctgcatgaccggcgccaaggtgtccatcaagctgcactccagcaagaacagcaccgcccacgtgcggtgcaagggcgacgagacagccttctctgtgctggaaggcgtgcacagctacaccgtgtccctgagcttcgaccacgccgtggtggatgagcagtgccaactgaactgtggcggccacgagtcccaagtgaccctgaagggaaacctgatcttcctggacgtgggatcctctggaggagctagcgtgctggcctccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagtccggaagc

SEQ ID NO: 21 - protein, 621 aa, Synthetic construct
CDEMVHADSKLVSCRQGSGNMKECVTTGRALLPAVNPGQEACLHFTAPGSPDSKCLKIKVKRINLKCKKSSSYFVPDARSRCTSVRRCRWAGDCQSGCPPHFTSNSFSDDWAGKMDRAGLGFSGCSDGCGGAACGCFNAAPSCIFWRKWVENPHGIIWKVSPCAAWVPSAVIELTMPSGEVRTFHPMSGIPTQVFKGVSVTYLGSDMEVSGLTDLCEIEELKSKKLALAPCNQAGMGVVGKVGEIQCSSEESARTIKKDGCIWNADLVGIELRVDDAVCYSKITSVEAVANYSAIPTTIGGLRFERSHDSQGKISGSPLDITAIRGSFSVNYRGLRLSLSEITATCTGEVTNVSGCYSCMTGAKVSIKLHSSKNSTAHVRCKGDETAFSVLEGVHSYTVSLSFDHAVVDEQCQLNCGGHESQVTLKGNLIFLDVGSSGGASVLASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 22 - DNA, 2094 bp, Synthetic construct
  source 1..2094: mol_type = other DNA
tgtgacgaaatggtgcacgccgacagcaaactggtgtcctgcagacagggctccggcaacatgaaggaatgcgtgaccaccggcagagccctgctgcctgctgtgaaccctggacaggaagcctgcctgcactttaccgcccctggcagccctgacagcaagtgcctgaagatcaaagtgaagcggatcaacctgaagtgcaagaaaagcagctcctacttcgtgcccgacgcccggtccagatgtaccagcgtgcggagatgtagatgggcaggcgattgccagagcggctgccctcctcacttcaccagcaacagcttcagcgacgactgggccggcaagatggacagagccggactgggctttagcggctgctctgatggatgtggcggagccgcctgcggctgctttaatgccgcccctagctgcatcttctggcggaaatgggtggaaaacccccacggcatcatctggaaggtgtccccatgtgccgcctgggtgccatctgccgtgatcgagctgacaatgcccagcggcgaagtgcggaccttccaccctatgagcggcatccccacccaggtgttcaagggcgtgtccgtgacatacctgggctccgacatggaagtgtccggcctgaccgacctgtgcgagattgaggaactgaaatccaagaagctggccctggccccctgtaaccaggccggaatgggagtcgtgggaaaagtgggcgagattcagtgcagctccgaggaaagcgcccggaccatcaagaaggacggctgcatctggaacgccgacctcgtgggaattgagctgagagtggatgacgccgtgtgttacagcaagatcaccagcgtggaagccgtggccaactacagcgccatccctaccacaatcggcggcctgagattcgagcggagccacgatagccagggcaagatcagcggaagccccctggacatcaccgccatcaggggcagctttagcgtgaactaccggggcctgagactgagcctgagcgagatcacagccacctgtaccggggaagtgaccaatgtgtccggctgctactcctgcatgaccggcgccaaggtgtccatcaagctgcactccagcaagaacagcaccgcccacgtgcggtgcaagggcgacgagacagccttctctgtgctggaaggcgtgcacagctacaccgtgtccctgagcttcgaccacgccgtggtggatgagcagtgccaactgaactgtggcggccacgagtcccaagtgaccctgaagggaaacctgatcttcctggacgtgcccaagttcgtggacggctcctacatgcagacctaccacagcaccgtgcccacaggcgccaacatccctagccctaccgactggctgaacgccctgttcggcaacggcctgagcagatggatcctgggcgtgatcggagtgctgctgggaggactggccctgttcttcctgatcatgagcctgttcaagctgggcaccaaacaggtgttccggtcccggacaaagctggccggatcctctggaggagctagcgtgctggcctccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagtccggaagc

SEQ ID NO: 23 - protein, 698 aa, Synthetic construct
CDEMVHADSKLVSCRQGSGNMKECVTTGRALLPAVNPGQEACLHFTAPGSPDSKCLKIKVKRINLKCKKSSSYFVPDARSRCTSVRRCRWAGDCQSGCPPHFTSNSFSDDWAGKMDRAGLGFSGCSDGCGGAACGCFNAAPSCIFWRKWVENPHGIIWKVSPCAAWVPSAVIELTMPSGEVRTFHPMSGIPTQVFKGVSVTYLGSDMEVSGLTDLCEIEELKSKKLALAPCNQAGMGVVGKVGEIQCSSEESARTIKKDGCIWNADLVGIELRVDDAVCYSKITSVEAVANYSAIPTTIGGLRFERSHDSQGKISGSPLDITAIRGSFSVNYRGLRLSLSEITATCTGEVTNVSGCYSCMTGAKVSIKLHSSKNSTAHVRCKGDETAFSVLEGVHSYTVSLSFDHAVVDEQCQLNCGGHESQVTLKGNLIFLDVPKFVDGSYMQTYHSTVPTGANIPSPTDWLNALFGNGLSRWILGVIGVLLGGLALFFLIMSLFKLGTKQVFRSRTKLAGSSGGASVLASGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 24 - DNA, 303 bp, Synthetic construct
  source 1..303: mol_type = other DNA
gtggcctgccagcccatgacccctatccccgaggaaacctttctggaactgaagtccttcagccagagcgagttccccgacatctgcaagatcgacggcatcgtgttcaaccagtgcgagggcgagagcctgccccagccttttgatgtggcctggatggacgtgggccactcccacaagatcatcatgcgcgagcacaagaccaaatgggtgcaggaaagcgtggtggtgtcttacggcggaatgagagtgcggcccaagtgctacggcttcagccggatgatggccaccctggaagtgaac

SEQ ID NO: 25 - protein, 101 aa, Synthetic construct
VACQPMTPIPEETFLELKSFSQSEFPDICKIDGIVFNQCEGESLPQPFDVAWMDVGHSHKIIMREHKTKWVQESVVVSYGGMRVRPKCYGFSRMMATLEVN

SEQ ID NO: 26 - DNA, 179 bp, Synthetic construct
  source 1..179: mol_type = other DNA
agcagcaaggacttcgtgtgctacaaagagggcaccggcccctgcagcgagagcgaggaaaagacctgcaagaccagcggctcctgcagaggcgacatgcagttctgcaaggtggccggatgcgagcacggcgaagaggccagcgaggccaagtgcagatgtagcctggtgcacaagcc

SEQ ID NO: 27 - protein, 59 aa, Synthetic construct
SSKDFVCYKEGTGPCSESEEKTCKTSGSCRGDMQFCKVAGCEHGEEASEAKCRCSLVHK

SEQ ID NO: 28 - DNA, 51 bp, Synthetic construct
  source 1..51: mol_type = other DNA
atggaaatcaaggtgctgtttgccctcatctgtattgctgttgctgaggca

SEQ ID NO: 29 - DNA, 21 bp, Synthetic construct
  source 1..21: mol_type = other DNA
aattcacatttgagggtagtt

SEQ ID NO: 30 - DNA, 23 bp, Synthetic construct
  source 1..23: mol_type = other DNA
tatccaaggaggatgacaataat

SEQ ID NO: 31 - RNA, 1521 nt, Synthetic construct
  source 1..1521: mol_type = other RNA
accggtcctatcatctgtgccggacccatccacagcaacaagagcgccgacatcccccatctgctgggctacagcgagaagatttgccagatcgaccggctgatccacgtgtcctcttggctgcggaaccacagccagttccagggctacgtgggacagagaggcggcagatcccaggtgtcctactacccagctgaaaacagctacagccggtggagcggcctgctgagcccttgtgatgctgactggctgggcatgctggtcgtgaagaaggccaagggcagcgacatgatcgtgcctggccctagctacaagggcaaggtgttcttcgagcggcccaccttcgacggctatgtgggctggggatgtggcagcggcaagagcagaacagagtccggcgagctgtgcagcagcgattctggcacaagctctggcctgctgcccagcgacagagtgctgtggattggcgacgtggcctgccagcccatgacccctatccccgaggaaacctttctggaactgaagtccttcagccagagcgagttccccgacatctgcaagatcgacggcatcgtgttcaaccagtgcgagggcgagagcctgccccagccttttgatgtggcctggatggacgtgggccactcccacaagatcatcatgcgcgagcacaagaccaaatgggtgcaggaaagcagcagcaaggacttcgtgtgctacaaagagggcaccggcccctgcagcgagagcgaggaaaagacctgcaagaccagcggctcctgcagaggcgacatgcagttctgcaaggtggccggatgcgagcacggcgaagaggccagcgaggccaagtgcagatgtagcctggtgcacaagcccggcgaggtggtggtgtcttacggcggaatgagagtgcggcccaagtgctacggcttcagccggatgatggccaccctggaagtgaacggatcctctggaggagctagcgtgctggcctccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagtccggaagc

SEQ ID NO: 32 - RNA, 51 nt, Synthetic construct
  source 1..51: mol_type = other RNA
atggaaatcaaggtgctgtttgccctcatctgtattgctgttgctgaggca

SEQ ID NO: 33 - DNA, 960 bp, Synthetic construct; identical to SEQ ID NO: 1
  source 1..960: mol_type = other DNA

SEQ ID NO: 34 - RNA, 960 nt, Synthetic construct
  source 1..960: mol_type = other RNA
accggtcctatcatctgtgccggacccatccacagcaacaagagcgccgacatcccccatctgctgggctacagcgagaagatttgccagatcgaccggctgatccacgtgtcctcttggctgcggaaccacagccagttccagggctacgtgggacagagaggcggcagatcccaggtgtcctactacccagctgaaaacagctacagccggtggagcggcctgctgagcccttgtgatgctgactggctgggcatgctggtcgtgaagaaggccaagggcagcgacatgatcgtgcctggccctagctacaagggcaaggtgttcttcgagcggcccaccttcgacggctatgtgggctggggatgtggcagcggcaagagcagaacagagtccggcgagctgtgcagcagcgattctggcacaagctctggcctgctgcccagcgacagagtgctgtggattggcgacgtggcctgccagcccatgacccctatccccgaggaaacctttctggaactgaagtccttcagccagagcgagttccccgacatctgcaagatcgacggcatcgtgttcaaccagtgcgagggcgagagcctgccccagccttttgatgtggcctggatggacgtgggccactcccacaagatcatcatgcgcgagcacaagaccaaatgggtgcaggaaagcagcagcaaggacttcgtgtgctacaaagagggcaccggcccctgcagcgagagcgaggaaaagacctgcaagaccagcggctcctgcagaggcgacatgcagttctgcaaggtggccggatgcgagcacggcgaagaggccagcgaggccaagtgcagatgtagcctggtgcacaagcccggcgaggtggtggtgtcttacggcggaatgagagtgcggcccaagtgctacggcttcagccggatgatggccaccctggaagtgaac

SEQ ID NO: 35 - DNA, 1521 bp, Synthetic construct; identical to SEQ ID NO: 16
  source 1..1521: mol_type = other DNA

SEQ ID NO: 36 - RNA, 1521 nt, Synthetic construct; identical to SEQ ID NO: 31
  source 1..1521: mol_type = other RNA

SEQ ID NO: 37 - DNA, 960 bp, Synthetic construct
  source 1..960: mol_type = other DNA
accgggccgatcatctgcgccgggccgatccactccaacaagtccgccgacatcccccacctgctgggctactccgagaagatctgccagatcgaccgcctgatccacgtgtccagctggctgcgcaaccactcgcagttccagggctacgtgggccagcgcggtggccgctcccaggtgagctactacccggccgagaacagctactcgcgctggtcgggcctgctgagcccctgcgacgccgactggctgggcatgctggtggtgaagaaggccaagggctcggacatgatcgtgccgggccccagctacaagggcaaggtgttcttcgagcgccccaccttcgacggctacgtgggctggggctgcggctccggcaagtcccgcaccgagtcgggcgagctgtgctcctcggactcgggcacctcctcgggcctgctgccctcggaccgcgtgctgtggatcggcgacgtggcctgccagcccatgacccccatcccggaggagacgttcctggagctgaagagcttctcccagagcgagttccccgacatctgcaagatcgacggcatcgtgttcaaccagtgcgagggcgagagcctgccccagcccttcgacgtggcctggatggacgtgggccacagccacaagatcatcatgcgcgagcacaagaccaagtgggtgcaggagtccagctccaaggacttcgtgtgctacaaagagggcaccggcccctgctcggagtcggaggagaagacctgcaagacctcgggcagctgccgcggcgacatgcagttctgcaaggtggccggctgcgagcacggcgaggaagcctcggaggccaagtgccgctgctccctggtgcacaagccgggcgaggtggtggtgtcctacggtggcatgcgcgtgcgcccgaagtgctacggcttcagccgcatgatggccaccctggaggtgaac

SEQ ID NO: 38 - RNA, 960 nt, Synthetic construct
  source 1..960: mol_type = other RNA
accgggccgatcatctgcgccgggccgatccactccaacaagtccgccgacatcccccacctgctgggctactccgagaagatctgccagatcgaccgcctgatccacgtgtccagctggctgcgcaaccactcgcagttccagggctacgtgggccagcgcggtggccgctcccaggtgagctactacccggccgagaacagctactcgcgctggtcgggcctgctgagcccctgcgacgccgactggctgggcatgctggtggtgaagaaggccaagggctcggacatgatcgtgccgggccccagctacaagggcaaggtgttcttcgagcgccccaccttcgacggctacgtgggctggggctgcggctccggcaagtcccgcaccgagtcgggcgagctgtgctcctcggactcgggcacctcctcgggcctgctgccctcggaccgcgtgctgtggatcggcgacgtggcctgccagcccatgacccccatcccggaggagacgttcctggagctgaagagcttctcccagagcgagttccccgacatctgcaagatcgacggcatcgtgttcaaccagtgcgagggcgagagcctgccccagcccttcgacgtggcctggatggacgtgggccacagccacaagatcatcatgcgcgagcacaagaccaagtgggtgcaggagtccagctccaaggacttcgtgtgctacaaagagggcaccggcccctgctcggagtcggaggagaagacctgcaagacctcgggcagctgccgcggcgacatgcagttctgcaaggtggccggctgcgagcacggcgaggaagcctcggaggccaagtgccgctgctccctggtgcacaagccgggcgaggtggtggtgtcctacggtggcatgcgcgtgcgcccgaagtgctacggcttcagccgcatgatggccaccctggaggtgaac

SEQ ID NO: 39 - DNA, 1521 bp, Synthetic construct
  source 1..1521: mol_type = other DNA
accgggccgatcatctgcgccgggccgatccactccaacaagtccgccgacatcccccacctgctgggctactccgagaagatctgccagatcgaccgcctgatccacgtgtccagctggctgcgcaaccactcgcagttccagggctacgtgggccagcgcggtggccgctcccaggtgagctactacccggccgagaacagctactcgcgctggtcgggcctgctgagcccctgcgacgccgactggctgggcatgctggtggtgaagaaggccaagggctcggacatgatcgtgccgggccccagctacaagggcaaggtgttcttcgagcgccccaccttcgacggctacgtgggctggggctgcggctccggcaagtcccgcaccgagtcgggcgagctgtgctcctcggactcgggcacctcctcgggcctgctgccctcggaccgcgtgctgtggatcggcgacgtggcctgccagcccatgacccccatcccggaggagacgttcctggagctgaagagcttctcccagagcgagttccccgacatctgcaagatcgacggcatcgtgttcaaccagtgcgagggcgagagcctgccccagcccttcgacgtggcctggatggacgtgggccacagccacaagatcatcatgcgcgagcacaagaccaagtgggtgcaggagtccagctccaaggacttcgtgtgctacaaagagggcaccggcccctgctcggagtcggaggagaagacctgcaagacctcgggcagctgccgcggcgacatgcagttctgcaaggtggccggctgcgagcacggcgaggaagcctcggaggccaagtgccgctgctccctggtgcacaagccgggcgaggtggtggtgtcctacggtggcatgcgcgtgcgcccgaagtgctacggcttcagccgcatgatggccaccctggaggtgaacggatcctctggaggagctagcgtgctggcctccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagtccggaagc

SEQ ID NO: 40 - RNA, 1521 nt, Synthetic construct
  source 1..1521: mol_type = other RNA
accgggccgatcatctgcgccgggccgatccactccaacaagtccgccgacatcccccacctgctgggctactccgagaagatctgccagatcgaccgcctgatccacgtgtccagctggctgcgcaaccactcgcagttccagggctacgtgggccagcgcggtggccgctcccaggtgagctactacccggccgagaacagctactcgcgctggtcgggcctgctgagcccctgcgacgccgactggctgggcatgctggtggtgaagaaggccaagggctcggacatgatcgtgccgggccccagctacaagggcaaggtgttcttcgagcgccccaccttcgacggctacgtgggctggggctgcggctccggcaagtcccgcaccgagtcgggcgagctgtgctcctcggactcgggcacctcctcgggcctgctgccctcggaccgcgtgctgtggatcggcgacgtggcctgccagcccatgacccccatcccggaggagacgttcctggagctgaagagcttctcccagagcgagttccccgacatctgcaagatcgacggcatcgtgttcaaccagtgcgagggcgagagcctgccccagcccttcgacgtggcctggatggacgtgggccacagccacaagatcatcatgcgcgagcacaagaccaagtgggtgcaggagtccagctccaaggacttcgtgtgctacaaagagggcaccggcccctgctcggagtcggaggagaagacctgcaagacctcgggcagctgccgcggcgacatgcagttctgcaaggtggccggctgcgagcacggcgaggaagcctcggaggccaagtgccgctgctccctggtgcacaagccgggcgaggtggtggtgtcctacggtggcatgcgcgtgcgcccgaagtgctacggcttcagccgcatgatggccaccctggaggtgaacggatcctctggaggagctagcgtgctggcctccggagagagccaggtgaggcagcagttcagcaaggacatcgagaagctgctgaacgagcaggtgaacaaggagatgcagagcagcaacctgtacatgagcatgagcagctggtgctacacccacagcctggacggcgccggcctgttcctgttcgaccacgccgccgaggagtacgagcacgccaagaagctgatcatcttcctgaacgagaacaacgtgcccgtgcagctgaccagcatcagcgcccccgagcacaagttcgagggcctgacccagatcttccagaaggcctacgagcacgagcagcacatcagcgagagcatcaacaacatcgtggaccacgccatcaagagcaaggaccacgccaccttcaacttcctgcagtggtacgtggccgagcagcacgaggaggaggtgctgttcaaggacatcctggacaagatcgagctgatcggcaacgagaaccacggcctgtacctggccgaccagtacgtgaagggcatcgccaagagcaggaagtccggaagc